(19) United States
(12) Patent Application Publication
(10) Pub. No.: US 2008/0039442 A1
(43) Pub. Date: Feb. 14, 2008

(54) Compounds With Kv4 Ion Channel Activity

(75) Inventors: Petra Blom, Destelbergen (BE); Jan Octaaf de Kerpel, Lede (BE); Eric Pierre Paul Rene Fourmaintraux, Lomme (FR); Titus Jan Kaletta, Merelbeke (BE); Dirk Leysen, Lokeren (BE)

Correspondence Address: WOLF GREENFIELD & SACKS, P.C., 600 ATLANTIC AVENUE, BOSTON, MA, 02210-2206, US

(73) Assignee: Devgen NV, Zwijnaarde (BE)

(21) Appl. No.: 11/793,559

(22) PCT Filed: Dec. 20, 2005
(86) PCT No.: PCT/EP05/13715

§ 371 Date: Jun. 19, 2007

Related U.S. Application Data
(60) Provisional application No. 60/637,660, filed on Dec. 21, 2004.
(60) Provisional application No. 60/686,913, filed on Jun. 3, 2005.

(30) Foreign Application Priority Data
Dec. 21, 2004 (EP) 04447288.4
Sep. 22, 2005 (EP) 05077173.2

(51) Int. Cl. A61K 31/343; A61K 31/357; A61K 31/381; A61K 31/404; A61K 31/4155; A61K 31/423; A61K 31/433; A61K 31/4525; A61K 31/47; A61K 31/4965; A61K 31/497; A61K 31/5377; A61K 31/55; A61P 25/00; A61P 9/00; C07D 209/42; C07D 215/48; C07D 231/10; C07D 233/18; C07D 241/02; C07D 263/58; C07D 265/30; C07D 285/06; C07D 307/82; C07D 307/85; C07D 307/87; C07D 319/16; C07D 333/56; C07D 333/70; C07D 405/02; C07D 409/12; C07D 413/02; C07D 493/00

(52) U.S. Cl. 514/217; 514/233.5; 514/255.05; 514/255.06; 514/311; 514/320; 514/361; 514/375; 514/406; 514/414; 514/419; 514/443; 514/444; 514/452; 514/464; 514/469; 514/470; 540/590; 544/153; 544/169; 544/376; 544/402; 546/196; 548/127; 548/222; 548/364.4; 548/454; 548/537; 549/366; 549/454; 549/462; 549/464; 549/467; 549/58

(57) ABSTRACT

The present invention relates to compounds that interact with ion channels. In particular, the invention relates to compounds having the structural Formula (I), (II), (III) or (IV), stereoisomers, tautomers, racemics, prodrugs, metabolites thereof, or a pharmaceutically acceptable salt and/or solvate thereof, Formula (I), (II), (III), (IV), wherein X¹, X², Y, Z, W, R¹, R⁸, R⁹, R¹⁰, L, A, z, and n have the meaning defined in claim 1. The invention also relates to methods for preparing said compounds, to pharmaceutical compositions comprising said compounds, and to the use of said compounds in methods for treatment of the human and animal body.

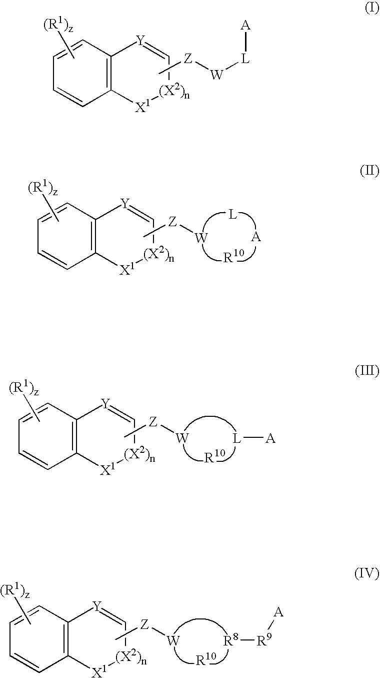

### FIELD OF THE INVENTION

The present invention relates to compounds that interact with ion channels.

In particular, the invention relates to compounds that interact with ion channels from the Kv family, and in particular from the Kv4 subfamily.

The invention also relates to methods for preparing said compounds, to pharmaceutical compositions that contain said compounds, and to the use of said compounds in methods for treatment of the human and animal body and/or to the use of said compounds in the preparation of such pharmaceutical compositions.

The compounds of the invention for example can be used in the prevention and/or treatment of conditions or diseases associated with ion channels, in particular in the prevention and/or treatment of conditions and diseases associated with ion channels of the Kv family, and more in particular in the prevention and/or treatment of conditions and diseases associated with ion channels of the Kv4 family.

Other aspects, embodiments, uses and advantages of the invention will become clear from the further description below.

### BACKGROUND TO THE INVENTION

Kv4 channels, as well as their encoding sequences, their biological function/activity and their disease associations have been described in the art, see for example Bahring et al., J. Biol. Chem., Vol. 276, no. 26, 23888-23894 (2001); Baldwin et al., Neuron 7: 471-483 (1991); Dixon et al., Circ. Res. 79: 659-688 (1996); Dilks et al., J. Neurophysiol. 81: 1974-1977 (1999); Kuo et al., Cell, Vol. 107, 801-813 (2001); Pak et al., Proc. Natl. Acad. Sci. USA 88; 4386-4390 (1991); Ohya et al., FEBS Lett. 420:47-53 (1997); Roberts and Tamkun, Proc. Natl. Acad. Sci. USA 88; 1798-1802; Rudy et al., Mol. Cell. Neurosci. 2; 89-102 (1991); Serodio et al., J. Neurophysiol, 75: 2174-2179 (1996); Serodio and Rudy, J. Neurophysiol. 79: 1081-1091 (1998); and Takimoto et al., Circ. Res. 81: 553-539 (1997), and the further references cited therein.

Generally, being voltage-gated potassium channels, Kv4 channels are inter alia involved in membrane depolarisation and repolarisation events, e.g. as part of and/or following neuronal firing and/or as part of the cycle of muscle contraction/relaxation.

In particular, and as mentioned in the above references, Kv4 channels are believed to be involved in the native A-type currents that are generated by various types of primary cells (Dilks et al., supra), in particular in muscle and neuronal cells. Kv4.2 and Kv4.3 transcripts have been found in most neurons, and in particular in CNS neurons (see Serodio and Rudy, supra, who discuss the distribution of Kv4 channels in rat brain); as well as in heart muscle (see Dixon et al. and by Serodio et al., both supra, who discuss the abundance and distribution of Kv4 transcripts in the hearts of rat, dog and human). It has also been found that, compared to Kv-type channels from other families such as Kv1-type channels, Kv4 channels activate and inactivate at subthreshold potentials, inactivate with time constants that change very little as a function of voltage (even at very negative potentials), and recover very fast from inactivation (see Rudy and Serodio, supra). In neuronal cells, and in particular in neurons in the brain, Kv4 channels are inter alia believed to play an important role in the modulation of the firing rate, action potential initiation, shaping burst pattern and postsynaptic signal integration (Dilks et al., and Bahring et al., supra), and are believed to be associated with the physiological states/disorders that result from such activity (Serodio and Rudy, supra).

In the heart, the Kv4 channels are inter alia believed to play a major role in the calcium-independent A-type currents in the cardiac muscle (the “transient outward current” or “Iₜₒ”), and in particular in the cardiac ventricular muscle, and are thus believed to be involved in early repolarization and hence the overall duration of the action potential and the length of the refractory period (Serodio and Rudy, supra). Because of this, Kv4 channels are believed to be associated with (the susceptibility to) cardiac disorders such as arrhythmia and other types of heart failure (Kuo et al., supra).

So far, three mammalian Kv4 genes—referred to as Kv4.1 (also known as mSha1), Kv4.2 (also known as RK5) and Kv4.3, respectively—have been cloned and characterized, i.e. from rat and dog (Dixon et al, Serodio et al., Ohya et al. and Takimoto et al., all supra) and from human (Dilks et al., and Bahring et al., supra; see also for example WO 98/42833 and U.S. Pat. No. 6,395,477).

The sequences of genes encoding mammalian Kv4 channels are also available from publicly accessible databases such as GenBank/NCBI, e.g. Kv4.1 from mouse (accession number NP_{—}032449 and A38372); Kv4.1 from human (accession number BAA96454, AAF65617 and AF65516); Kv4.2 from mouse (accession number NP_{—}062671 and AAD16972), Kv4.2 from rat (accession number NP_{—}113918); Kv4.2 from human (accession number AAD22053 and CAB56841); Kv4.3 from mouse (accession numbers NM_{—}019931 and AF384170), Kv4.3 from rat (accession number U42975) and Kv4.3 from human (accession number XM_{—}052127).

The above references also indicate that further channels from the Kv4 family may be identified and cloned in future, for example from neurons in the brain that show Kv4-like subthreshold-operating A channels, but do not show abundant expression of Kv4.1, Kv4.2 and/or Kv4.3 transcripts (see Serodio and Rudy, supra) or other suitable tissues/cells.

As mentioned above, the Kv4 channels in mammals also have a high degree of sequence identity (>70%) with, and thus are considered closely related to, the ShaI-like gene product, which encodes a potassium channel in Drosophila melanogaster (see Baldwin et al, supra, and also WO 01/58952).

An assay for determining the influence of a compound on Kv channels, in which a transgenic line of Caenorhabditis elegans expressing a heterologous Kv channel, such as a human Kv4.3 channel, is used, is described in the International application WO 03/097682 by Applicant. Other assays and techniques for determining the influence of a test compound on ion channels in general, and on a Kv channel in particular, such as FLIPR-techniques and use of oocytes, will be clear to the skilled person, and are also mentioned in WO 03/097682. Such assays can be used to determine whether a compound “interacts with” such an ion channel. As mentioned below and for the purposes of the present description and attached claims, a compound is considered to “interact with” an ion channel, such as an ion channel of the Kv family and in particular of the Kv4 subfamily, if such a compound acts as an antagonist of said ion channel and/or of the biological function(s) and/or pathways associated with said ion channels, and in particular if such a compound can fully or partially “block” such an ion channel.

In view of the biological functions and disease associations mentioned above, compounds that interact with ion channels can find use as pharmaceutically active agents, in particular for the prevention and/or treatment of diseases and disorders associated with the ion channels with which the compound interact. By means of non-limiting example, compounds that interact with ion channels from the Kv 4 subfamily, and in particular with Kv4.3 ion channels (e.g. the compounds as further described herein below) could be used in (the preparation of pharmaceutical compositions for) the prevention and/or treatment of cardiac disorders such as arrhythmia, hypertension-induced heart disorders such as hypertension-induced cardiac hypertrophy (e.g. ventricular hypertrophy), and disorders of the nervous system such as epilepsy, stroke, traumatic brain injury, anxiety, insomnia, spinal cord injury, encephalomyelitis, multiple sclerosis, demyelinating disease, Alzheimer's disease and Parkinson's syndrome.

A major drawback of some of the known compounds involves that the drugs do not work in a selective manner, i.e. they do not select between different ion channels. For instance many of these compounds also block a potassium channel called the human ether-á-go-go related gene (hERG) potassium channel. Compounds that block this channel with high potency may cause reactions which are fatal. This undesired blockade can cause acquired long QT syndrome, a disorder that puts patients at risk for life-threatening arrhythmias. Cardiac arrhythmias are the leading cause of sudden death in the United States, according to the American Heart Association. The FDA now requires that every drug be assayed for hERG block before it is approved. Even medicines that might be beneficial for the vast majority of patients do not make it to the market—or have been pulled from the market—if they block hERG.

Thus, in addition to being able to modulate a particular Kv channel, it is desirable to find compounds that are selective to Kv channel when compared to the hERG channel. Thus, there is a need to find compounds that modulate the Kv channel, while not inhibiting the hERG channel.

There remains an urgent need in the art for finding new compounds, which overcome the above-mentioned drawbacks.

It is therefore an object of the invention to provide compounds that interact with ion channels, in particular with ion channels from the Kv family, more in particular with ion channels from the Kv4 subfamily, and especially with Kv4.3 channels, in particular in vertebrates, more in particular in warm-blooded animals, even more in particular in mammals, and especially in human beings. It is a further object of the present invention to provide compounds that interact with ion channels, in particular Kv ion channels and which are selective to Kv ion channels when compared to the hERG channel.

### SUMMARY OF THE INVENTION

In a first aspect the present invention relates to compounds of Formula I, II, III or IV, stereoisomers, tautomers, racemics, prodrugs, metabolites thereof, or a pharmaceutically acceptable salt and/or solvate thereof, 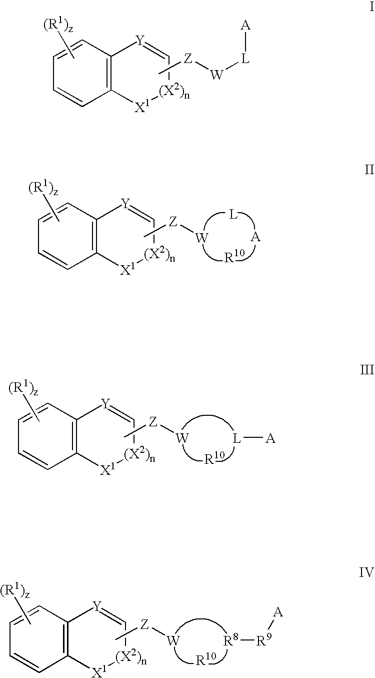
wherein X¹ is a heteroatom selected from —O—, —S—, —N═, or —N(R³)—, wherein R³ is selected from alkyl, aralkyl or alkylcarbonyl, wherein X² is selected from ═C—, ═CH— or —CH₂—,
wherein n is an integer selected from 0 or 1,
wherein Y is selected from C, —C(R⁵)— or N, wherein R⁵ is selected from hydrogen, amino, alkyl, hydroxyl, alkylamino, heteroaryl, alkylcarbonyloxy, alkylamidyl, or alkylaminocarbonylamino,
wherein Z is selected from —C(═O)—, —CH₂—, or —NH—,
wherein W is selected from —C(═O)—, —N(R²)—, —N(R²)—NH—, —C(═O)—NH—, —CH═, —O— or —CH₂—, in formula I, and W is selected from N, or CH in formula II, III or IV,
wherein R¹ is selected from hydrogen, halogen, hydroxy, nitro, amino, azido, cyano, or alkyl, cycloalkyl, alkylamino, alkoxy, carboxy, alkylaminocarbonyl, alkylcarbonyl, heterocyclyl-alkyl, heteroarylalkyl, alkoxycarbonyl, aminocarbonyl, alkylamino(alkylsubstituted)alkyl, alkylcarbonylaminoalkyl or alkylthio, each optionally substituted by one or more substitutent, wherein z is an integer selected from 1, 2, 3 or 4,
wherein R² is selected from hydrogen, alkyl, cycloalkyl, alkenyl aryl, aminocarbonyl, haloalkyl, aralkyl, cycloalkylalkyl, acyl or alkynyl,
wherein A is selected from aryl, cycloalkyl, heterocyclyl and heteroaryl, each optionally substituted by one or more substituents selected from halogen, hydroxy, nitro, azido, hydrazino, cyano, alkyl, aryl, heteroarylalkyl, 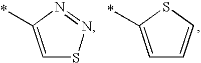
cycloalkyl, acyl, alkylamino, alkylaminocarbonyl, —SO₂R¹⁵, alkylcarbonyloxy, fused heterocyclyl, haloalkyl, alkylcarbonyl, aryloxy, arylcarbonyl, haloalkoxy, alkoxy, alkylthio, carboxy, acylamino, alkyl esters, carbamate, alkyloxycarbonyl, thioamide, urea, or sulphonamide, wherein R¹⁵ is alkyl, alkylamino or cycloalkyl,
wherein L is a linking group selected from a single bond, a group of formula —R⁸—R⁹—, alkylyn, alkenylyl, cycloalkylene, —NH—(C(R⁴)(R⁴))_{q}—, —(C(R⁴)(R⁴))_{q}—, —(C(R⁴)(R⁴))ᵥ—O—(C(R⁴)(R⁴))_{w}—, —(C(R⁴)(R⁴))ᵥ—N—(C(R⁴)(R⁴))_{w}—, —(C(R⁴)(R⁴))ᵥ—(C(R⁴))_{w}═, —(C(R⁴)(R⁴))_{q}—(CO)—, or cycloalkylenoxyalkylene, wherein —(C(R⁴)(R⁴))_{q}—, (C(R⁴)(R⁴))_{w} and —(C(R⁴)(R⁴))ᵥ— are each independently aliphatic or form a cycloalkyl, wherein each R⁴ is independently selected from hydrogen, alkyl, carboxy, hydroxyl, hydroxyalkyl, alkoxyalkyl, alkylamino, alkylaminoalkyl or alkyloxycarbonyl; q is an integer selected from 0, 1, 2, 3, 4, 5 or 6; v is an integer selected from 0, 1, 2, 3, 4, 5 or 6 and w is an integer selected from 0, 1, 2, 3, 4, 5 or 6,
wherein R⁸ is alkylyn, —(C(R⁴)(R⁴))ₚ—C(R¹⁴) or —(C(R⁴)(R⁴))ₚ—C(R⁴)═C, wherein R⁹ is selected from a single bond, —(C(R⁴)(R⁴))_{q}—, or —C(═O)—, wherein R¹⁴ is selected from hydrogen, hydroxyl or alkyl, wherein p is an integer selected from 0, 1, 2, or 3, and
wherein R¹⁰ is selected from —(C(R⁴)(R⁴))ₘ—, —(C(R⁴)(R⁴))ₘ—C(═O)O—(C(R⁴)(R⁴))_{q}—, or —(C(R⁴)(R⁴))ₘ—N(R¹²)—(C(R⁴)(R⁴))_{q}—, wherein m is an integer selected from 1, 2, 3, 4, 5 or 6, wherein R¹² is selected from hydrogen, alkyl, aryl, arylalkyl, or alkylcarbonyl.

In a second aspect, the present invention relates to a method for synthesizing a compound having the structural Formula I, II, III or IV comprising the step of condensing a compound of Formula LXIV: 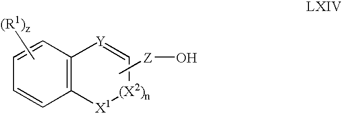
with a compound of Formula LXV, LXVI, LXVII or LXVIII: 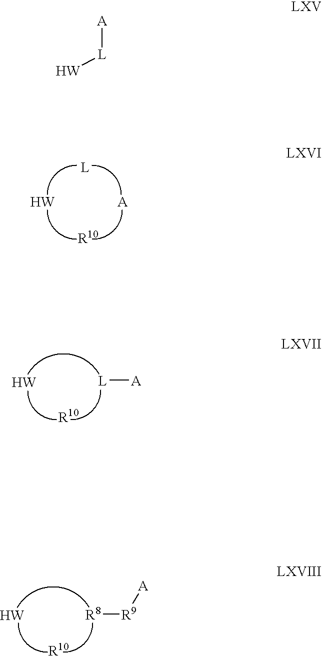
thereby obtaining a compound of Formula I, II, III or IV 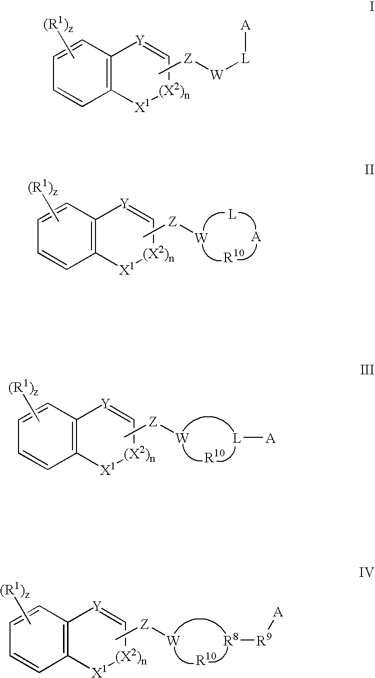
wherein R¹, z, X¹, X², W, Y, Z, n, L, A, R⁸, R⁹ and R¹⁰ have the same meaning as that defined hereinabove.

It was surprisingly found that the compounds of the invention interact with ion channels as shown in the examples below, in particular with ion channels from the Kv family, more in particular with ion channels from the Kv4 subfamily, and especially with Kv4.3 channels. Kv4.3 ion channels are associated to various conditions or diseases. In a further aspect, the present invention provides a compound of Formula I, II, III or IV for use as a medicament.

The compounds of the present invention are particularly useful for the preparation of a medicament in the prevention and/or treatment of conditions or diseases associated with ion channels of the Kv4 family. Non-limiting examples of said conditions or diseases associated with ion channels of the Kv4 family can be selected from the group comprising cardiac disorders including arrhythmia, hypertension-induced heart disorders including hypertension-induced cardiac hypertrophy, disorders of the nervous system including epilepsy, stroke, traumatic brain injury, anxiety, insomnia, spinal cord injury, encephalomyelitis, multiple sclerosis, demyelinating disease, Alzheimer's disease and Parkinson's syndrome. In an embodiment, the present invention provides for the use of a compound of the invention for the preparation of a medicament for treating cardiac disorders. In another embodiment, the present invention provides for the use of a compound of the invention for the preparation of a medicament for treating disorders of the nervous system.

In yet another aspect, the present invention provides a pharmaceutical composition comprising a pharmaceutically acceptable excipient and a therapeutically effective amount of a compound according to the invention. Said composition is particularly useful in the prevention and/or treatment of conditions or diseases associated with ion channels of the Kv4 family such as the one cited herein. Said composition is particularly suited for example in the treatment of cardiac disorders and disorders of the nervous system.

It was also surprisingly found that the compounds of the present invention interact with ion channels of the Kv1 subfamily, and especially with Kv1.5 ion channels.

The present invention also provides a method of treating cardiac disorders comprising administrating to an individual in need of such treatment a pharmaceutical composition to the invention. In another embodiment, the present invention provides a method of treating disorders of the nervous system comprising administrating to an individual in need of such treatment a pharmaceutical composition to the invention.

### DESCRIPTION OF THE INVENTION

Thus, in a first aspect the present invention relates to compounds of Formula I, II, III or IV, 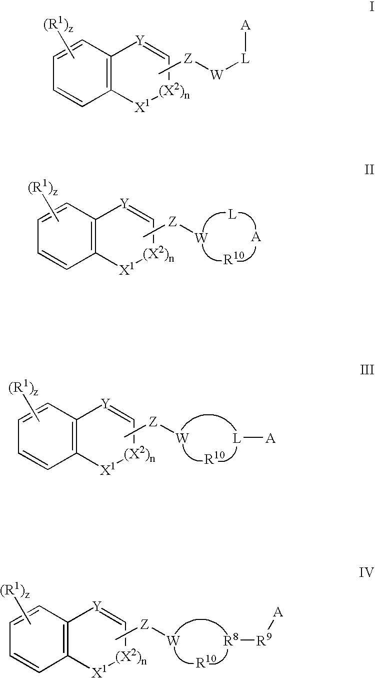
stereoisomers, tautomers, racemics, prodrugs, metabolites thereof, or a pharmaceutically acceptable salt and/or solvate thereof.

In an embodiment, X¹ is a hetero-atom selected from —O—, —S—, —N—, or —N(R³)—, wherein R³ is selected from alkyl or alkylcarbonyl, X² is selected from C, ═CH— or —CH₂—, and n is an integer selected from 0 or 1. In another embodiment, X¹ is oxygen and n is 0. In another embodiment, X¹ is —N(R³)—, n is 0 and wherein R³ has the same meaning as that defined hereinabove. In yet another embodiment, X¹ is —N═ and X² is ═CH— and n is 1. In yet another embodiment, Y is N and X′ is —N(R¹³)—, wherein R¹³ is selected from hydrogen, alkyl, aralkyl or alkylcarbonyl.

Z is selected from —C(═O)—, —CH₂—, or —NH— and W is selected from —C(═O)—, —N(R²)—, —N(R²)—NH—, —C(═O)—NH—, —CH═, —O— or —CH₂— in formula I, and W is selected from N, or CH in formula II, III or IV.

R¹ can be selected from hydrogen, halogen, hydroxy, nitro, amino, azido, cyano, or alkyl, cycloalkyl, alkylamino, alkoxy, carboxy, alkylaminocarbonyl, alkylcarbonyl, heterocyclylalkyl, alkylamino(alkylsubstituted)alkyl, heteroarylalkyl, alkoxycarbonyl, aminocarbonyl, alkylcarbonylaminoalkyl or alkylthio, each optionally substituted by one or more substituents, for example 1, 2 or 3 substituents, wherein the substituents can be the same as that described herein for “substituted alkyl”. According to the present invention, z is an integer between selected from 1, 2, 3 or 4, for example z can be 1, 2 or 3.

R² can be selected from hydrogen or alkyl, cycloalkyl, cyanoalkyl, alkenyl aryl, aminocarbonyl, haloalkyl, aralkyl, cycloalkylalkyl, acyl or alkynyl, each optionally substituted by 1, 2 or 3 substituents selected from alkyl, aryl, halogen, haloalkyl, haloalkoxy. For example, R² can be hydrogen, aryl, aralkyl, alkyl cyanoalkyl or cycloalkyl group. In an embodiment R² is a hydrogen, phenyl, benzyl, C₁-C₆ cyanoalkyl or C₁-C₆ alkyl group and for example a hydrogen, phenyl, benzyl, a C₁-C₄ cyanoalkyl or a C₁-C₄ alkyl group.

A can be an optionally substituted or polysubstituted aryl, cycloalkyl, heterocyclyl and heteroaryl. A is either unsubstituted or substituted by 1, 2, 3, 4 or 5, preferably 1, 2, or 3 and most preferably 1 or 2 substituents. Suitable substituents for A are not limited to halogen, hydroxy, nitro, azido, hydrazino, cyano, alkyl, aryl, 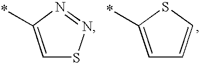
heteroarylalkyl, cycloalkyl, acyl, alkylamino, alkylaminocarbonyl, alkylcarbonyloxy, fused heterocyclyl, —SO₂R¹⁵, haloalkyl, alkylcarbonyl, aryloxy, alkyloxycarbonyl, arylcarbonyl, haloalkoxy, alkoxy, thiol, alkylthio, carboxy, acylamino, alkyl esters, carbamate, thioamide, urea, or sulphonamide, wherein R¹⁵ is alkyl, alkylamino or cycloalkyl.

L is a linking group and can be selected from a single bond, a group of formula —R⁸—R⁹—, alkylyn, alkenylyl, cycloalkylene, —NH—(C(R⁴)(R⁴))_{q}—, —(C(R⁴)(R⁴))_{q}—, —(C(R⁴)(R⁴))ᵥ—O—(C(R⁴)(R⁴))_{w}—, —(C(R⁴)(R⁴))ᵥ—(C(R⁴))_{w}═, —(C(R⁴)(R⁴))ᵥ—N—(C(R⁴)(R⁴))_{w}—, —(C(R⁴)(R⁴))_{q}—(C═O), or cycloalkylenoxyalkylene, wherein —(C(R⁴)(R⁴))_{q}—, (C(R⁴)(R⁴))_{w} and —(C(R⁴)(R⁴))ᵥ— are each independently aliphatic or form a cycloalkyl, wherein each R⁴ is independently selected from hydrogen, hydroxyl, alkyl, carboxy, hydroxyalkyl, alkylaminoalkyl, alkoxyalkyl, alkylamino, or alkyloxycarbonyl; q is an integer selected from 0, 1, 2, 3, 4, 5 or 6; v is an integer selected from 0, 1, 2, 3, 4, 5 or 6 and w is an integer selected from 0, 1, 2, 3, 4, 5 or 6, wherein R⁸ is alkylyn, —(C(R⁴)(R⁴))ₚ—C(R¹⁴) or —(C(R⁴)(R⁴))ₚ—C(R⁴)═C, wherein R⁹ is selected from a single bond, —(C(R⁴)(R⁴))_{q}—, or —C(═O)—, wherein R¹⁴ is selected from hydrogen, hydroxyl or alkyl, wherein p is an integer selected from 0, 1, 2 or 3.

R¹⁰ in formula II, III and IV can be selected from —(C(R⁴)(R⁴))ₘ—, —(C(R⁴)(R⁴))ₘ—C(═O)O—(C(R⁴)(R⁴))_{q}—, or —(C(R⁴)(R⁴))ₘ—N(R¹²)—(C(R⁴)(R⁴))_{q}—, wherein m is an integer selected from 1, 2, 3, 4, 5 or 6, wherein R¹² is selected from hydrogen, alkyl, aryl, arylalkyl, or alkylcarbonyl.

When describing the compounds of the invention, the terms used are to be construed in accordance with the following definitions, unless a context dictates otherwise:

The term “alkyl” by itself or as part of another substituent, refers to a straight or branched saturated hydrocarbon group joined by single carbon-carbon bonds having 1 to 10 carbon atoms, for example 1 to 8 carbon atoms, for example 1 to 6 carbon atoms, preferably 1 to 4 carbon atoms. When a subscript is used herein following a carbon atom, the subscript refers to the number of carbon atoms that the named group may contain. Thus, for example, C₁₋₄alkyl means an alkyl of one to four carbon atoms. Examples of alkyl groups are methyl, ethyl, propyl, isopropyl, butyl, isobutyl, sec-butyl, tert-butyl, 2-methylbutyl, pentyl iso-amyl and its isomers, hexyl and its isomers, heptyl and its isomers and octyl and its isomers.

The term “optionally substituted alkyl” refers to an alkyl group optionally substituted with one or more substituents (for example 1 to 4 substituents, or 1 to 2 substituents) at any available point of attachment. Non-limiting examples of such substituents include halogen, hydroxy, oxo, nitro, amino, oximes, imines, azido, hydrazinos, cyano, alkyl, aryl, heteroaryl, cycloalkyl, acyl, alkylamino, alkoxy, thiol, alkylthio, carboxylic acid, acylamino, alkyl esters, carbamates, thioamides, urea, sulphonamides and the like.

When the term “alkyl” is used as a suffix following another term, as in “hydroxyalkyl,” this is intended to refer to an alkyl group, as defined above, being substituted with one or two (preferably one) substituent(s) selected from the other, specifically-named group, also as defined herein. “Alkoxyalkyl” refers to an alkyl group substituted with one to two of OR′, wherein R′ is alkoxy as defined below. For example, “aralkyl” or “(aryl)alkyl” refers to a substituted alkyl group as defined above wherein at least one of the alkyl substituents is an aryl as defined below, such as benzyl.

The term “hydroxyalkyl” refers to a —R^{a}—OH group wherein R^{a} is alkylene as defined herein. For example, “hydroxyalkyl” includes 2-hydroxyethyl, 1-(hydroxymethyl)-2-methylpropyl, 3,4-dihydroxybutyl, and so forth.

The term “cycloalkyl” by itself or as part of another substituent, includes all saturated or partially saturated (containing 1 or 2 double bonds) hydrocarbon groups containing 1, 2 or 3 rings, including monocyclic, bicyclic or polycyclic alkyl groups wherein each cyclic moiety has from 3 to 8 carbon atoms, for example 3 to 7 carbon atoms, for example 3 to 6 carbon atoms, for example 3 to 5 carbon atoms. The further rings of multi-ringcycloalkyls may be either fused, bridged and/or joined through one or more spiro atoms. Examples of monocyclic cycloalkyl radicals include cyclopropyl, cyclobutyl, cyclopentyl, cyclohexyl, cycloheptyl, cyclooctyl and the like. Examples of polycyclic cycloalkyl radicals include decahydronaphthyl, bicyclo[5.4.0]undecyl, adamantyl, and the like. An “optionally substituted cycloalkyl” refers to a cycloalkyl having optionally one or more substituents (for example 1, 2 or 3 substituents, or 1 to 2 substituents), selected from those defined above for substituted alkyl. When the suffix “ene” is used in conjunction with a cyclic group, this is intended to mean the cyclic group as defined herein having two single bonds as points of attachment to other groups.

The term “alkenyl” by itself or as part of another substituent, refers to a straight or branched alkyl chain containing at least one unsaturation in the form of a single carbon to carbon double bond and having 2 to 10 carbon atoms, for example 2 to 8 carbon atoms, preferably 2 to 6 carbon atoms, more preferably 2 to 4 carbon atoms. Examples of alkenyl groups are ethenyl, 2-propenyl, 2-butenyl, 3-butenyl, 2-pentenyl and its isomers, 2-hexenyl and its isomers, 2-heptenyl and its isomers, 2-octenyl and its isomers, 2,4-pentadienyl and the like. An optionally substituted alkenyl refers to an alkenyl having optionally one or more substituents (for example 1, 2 or 3 substituents, or 1 to 2 substituents), selected from those defined above for substituted alkyl.

The term “alkynyl” by itself or as part of another substituent, refers to a straight or branched alkyl chain containing at least one unsaturation in the form of a single carbon to carbon triple bond and having 2 to 10 carbon atoms, for example 2 to 8 carbon atoms, preferably 2 to 6 carbon atoms, more preferably 2 to 4 carbon atoms. Examples alkynyl groups are ethynyl, 2-propynyl, 2-butynyl, 3-butynyl, 2-pentynyl and its isomers, 2-hexynyl and its isomers, 2-heptynyl and its isomers, 2-octynyl and its isomers and the like. An optionally substituted alkynyl refers to an alkynyl having optionally one or more substituents (for example 1, 2, 3 or 4 substituents, or 1 to 2 substituents), selected from those defined above for substituted alkyl.

Where alkyl groups as defined are divalent, i.e., with two single bonds for attachment to two other groups, they are termed “alkylene” groups. Non-limiting examples of alkylene groups includes methylene, ethylene, methylmethylene, trimethylene, propylene, tetramethylene, ethylethylene, 1,2-dimethylethylene, pentamethylene and hexamethylene. Similarly, where alkenyl groups as defined above and alkynyl groups as defined above, respectively, are divalent radicals having single bonds for attachment to two other groups, they are termed “alkenylene” and “alkynylene” respectively.

Where alkyl groups as defined are trivalent, i.e., with three single bonds for attachment to three other groups, they are termed “alkylyne” or “alkyline” groups. Non-limiting example of such alkylyne include, methine, 1,1,2-ethyline, and the like.

Where alkenyl groups as defined are trivalent, i.e., with three single bonds for attachment to three other groups, they are termed “alkenylyne” or “alkenyline” groups.

The term “aryl” as used herein by itself or as part of another group refers but is not limited to 5 to 14 carbon-atom homocyclic (i.e., hydrocarbon) monocyclic, bicyclic or tricyclic aromatic rings or ring systems containing 1 to 4 rings which are fused together or linked covalently, typically containing 5 to 8 atoms; at least one of which is aromatic. The aromatic ring may optionally include one to three additional rings (either cycloalkyl, heterocyclyl or heteroaryl) fused thereto.

Non-limiting examples of aryl comprise phenyl, biphenylyl, biphenylenyl, 5- or 6-tetralinyl, 1-, 2-, 3-, 4-, 5-, 6-, 7- or 8-azulenyl, 1- or 2-naphthyl, 1-, 2- or 3-indenyl, 1-, 2- or 9-anthryl, 1- 2-, 3-, 4- or 5-acenaphtylenyl, 3-, 4- or 5-acenaphtenyl, 1-, 2-, 3-, 4- or 10-phenanthryl, 1- or 2-pentalenyl, 1,2-, 3- or 4-fluorenyl, 4- or 5-indanyl, 5-, 6-, 7- or 8-tetrahydronaphthyl, 1,2,3,4-tetrahydronaphthyl, 1,4-dihydronaphthyl, dibenzo[a,d]cylcoheptenyl, 1-, 2-, 3-, 4- or 5-pyrenyl.

The aryl ring can optionally be substituted by one or more substituents. An “optionally substituted aryl” refers to an aryl having optionally one or more substituents (for example 1, 2, 3, 4, or 5 substituents, or 1, 2 or 3 substituents) at any available point of attachment. Non-limiting examples of such substituents are selected from halogen, hydroxy, oxo, nitro, amino, azido, hydrazino, cyano, alkyl, aryl, heteroaryl, 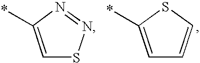
heteroarylalkyl, cycloalkyl, acyl, alkylamino, alkylaminocarbonyl, —SO₂R¹⁵, alkylcarbonyloxy, fused heterocyclyl, haloalkyl, alkylcarbonyl, alkyloxycarbonyl, aryloxy, arylcarbonyl, haloalkoxy, alkoxy, thiol, alkylthio, haloaryl, carboxy, acylamino, alkyl esters, carbamate, thioamide, urea, or sulphonamide, and the like, wherein R¹⁵ is alkyl, alkylamino or cycloalkyl.

The term “aryloxy” as used herein denotes a group —O-aryl, wherein aryl is as defined above.

The term “aroyl” as used herein denotes a group —C(O)-aryl, wherein aryl is as defined above.

The term “heteroaryl” as used herein by itself or as part of another group refers but is not limited to 5 to 12 carbon-atom aromatic rings or ring systems containing 1, 2 or 3 rings which are fused together or linked covalently, typically containing 5 to 8 atoms; at least one of which is aromatic in which one or more carbon atoms in one or more of these rings can be replaced by oxygen, nitrogen or sulfur atoms where the nitrogen and sulfur heteroatoms may optionally be oxidized and the nitrogen heteroatoms may optionally be quaternized. Such rings may be fused to an aryl, cycloalkyl, heteroaryl or heterocyclyl ring. An “optionally substituted heteroaryl” refers to a heteroaryl having optionally one or more substituents (for example 1, 2, 3 or 4 substituents, or 1, 2 or 3 substituents), selected from those defined above for substituted aryl.

Non-limiting examples of heteroaryl can be 2- or 3-furyl, 2- or 3-thienyl (thiophen-yl), 1-, 2- or 3-pyrrolyl, 1-, 2-, 4- or 5-imidazolyl, 1-, 3-, 4- or 5-pyrazolyl, 3-, 4- or 5-isoxazolyl, 2-, 4- or 5-oxazolyl, 3-, 4- or 5-isothiazolyl, 2-, 4- or 5-thiazolyl, 1,2,3-triazol-1-, -2-, -4- or -5-yl, 1,2,4-triazol-1-, -3-, -4- or -5-yl, 1,2,3-oxadiazol-4- or -5-yl, 1,2,4-oxadiazol-3- or -5-yl, 1,2,5-oxadiazolyl, 1,3,4-oxadiazolyl, 1,2,3-thiadiazol-4- or -5-yl, 1,2,4-thiadiazol-3- or -5-yl, 1,2,5-thiadiazol-3- or -4-yl, 1,3,4-thiadiazolyl, 1- or 5-tetrazolyl, 2-, 3- or 4-pyridyl, 3- or 4-pyridazinyl, 2-, 4-, 5- or 6-pyrimidinyl, 2-, 3-, 4-, 5- 6-2H-thiopyranyl, 2-, 3- or 4-4H-thiopyranyl, 2-, 3-, 4-, 5-, 6- or 7-benzofuryl, 1-, 3-, 4- or 5-isobenzofuryl, 2-, 3-, 4-, 5-, 6- or 7-benzothienyl, 1-, 3-, 4- or 5-isobenzothienyl, 1-, 2-, 3-, 4-, 5-, 6- or 7-indolyl, 2- or 3-pyrazinyl, 1,4-oxazin-2- or -3-yl, 1,4-dioxin-2- or -3-yl, 1,4-thiazin-2- or -3-yl, 1,2,3-triazinyl, 1,2,4-triazinyl, 1,3,5-triazin-2-, -4- or -6-yl, thieno[2,3-b]furan-2-, -3-, -4-, or -5-yl, 1-, 2-, 4- or 5-benzimidazolyl, 1-, 3-, 4-, 5-, 6- or 7-benzopyrazolyl, 3-, 4-, 5-, 6- or 7-benzisoxazolyl, 2-, 4-, 5-, 6- or 7-benzoxazolyl, 3-, 4-, 5-, 6- or 7-benzisothiazolyl, 2-, 4-, 5-, 6- or 7-benzothiazolyl, 1-, 2-thianthrenyl, 3-, 4- or 5-isobenzofuranyl, 1-, 2-, 3-, 4- or 9-xanthenyl, 1-, 2-, 3- or 4-phenoxathiinyl, 2-, 3-pyrazinyl, 1-, 2-, 3-, 4-, 5-, 6-, 7- or 8-indolizinyl, 2-, 3-, 4- or 5-isoindolyl, 1-, 2-, 3-, 4-, 5-, 6- or 7-indazolyl, 2-, 6-, 7- or 8-purinyl, 4-, 5- or 6-phthalazinyl, 2-, 3- or 4-naphthyridinyl, 2-, 5- or 6-quinoxalinyl, 2-, 4-, 5-, 6-, 7- or 8-quinazolinyl, 1-, 2-, 3- or 4-quinolizinyl, 2-, 3-, 4-, 5-, 6-, 7-, or 8-quinolinyl(quinolyl), 2-, 4-, 5-, 6-, 7- or 8-quinazolyl, 1-, 3-, 4-, 5-, 6-, 7- or 8-isoquinolinyl(isoquinolyl), 3-, 4-, 5-, 6-, 7- or 8-cinnolinyl, 2-, 4-, 6- or 7-pteridinyl, 1-, 2-, 3-, 4- or 9-carbazolyl, 1-, 2-, 3-, 4-, 5-, 6-, 7-, 8- or 9-carbolinyl, 1-, 2-, 3-, 4-, 5-, 6-, 7-, 8-, 9- or 10-phenanthridinyl, 1-, 2-, 3- or 4-acridinyl, 1-, 2-, 3-, 4-, 5-, 6-, 7-, 8- or 9-perimidinyl, 2-, 3-, 4-, 5-, 6-, 7-, 8-, 9- or 10-(1,7)phenanthrolinyl, 1- or 2-phenazinyl, 1-, 2-, 3-, 4-, or 10-phenothiazinyl, 3- or 4-furazanyl, 1-, 2-, 3-, 4-, or 10-phenoxazinyl, azepinyl, diazepinyl, dibenzo[b,f]azepinyl, dioxanyl, thietanyl, oxazolyl dibenzo[a,d]cylcoheptenyl, or additionally substituted derivatives thereof.

The terms “heterocyclyl” or “heterocyclo” as used herein by itself or as part of another group refer to non-aromatic, fully saturated or partially unsaturated cyclic groups (for example, 3 to 13 member monocyclic, 7 to 17 member bicyclic, or 10 to 20 member tricyclic ring systems, or containing a total of 3 to 10 ring atoms) which have at least one heteroatom in at least one carbon atom-containing ring. Each ring of the heterocyclic group containing a heteroatom may have 1, 2, 3 or 4 heteroatoms selected from nitrogen atoms, oxygen atoms and/or sulfur atoms, where the nitrogen and sulfur heteroatoms may optionally be oxidized and the nitrogen heteroatoms may optionally be quaternized. The heterocyclic group may be attached at any heteroatom or carbon atom of the ring or ring system, where valence allows. The rings of multi-ring heterocycles may be fused, bridged and/or joined through one or more spiro atoms. An “optionally substituted heterocyclyl” refers to a heterocyclic having optionally one or more substituents (for example 1, 2, 3 or 4 substituents, or 1 to 2 substituents), selected from those defined above for substituted aryl.

Exemplary heterocyclic groups include piperidinyl, azetidinyl, imidazolinyl, imidazolidinyl, isoxazolinyl, oxazolidinyl, isoxazolidinyl, thiazolidinyl, isothiazolidinyl, piperidyl, succinimidyl, 3H-indolyl, indolinyl, isoindolinyl, chromenyl, isochromanyl, xanthenyl, 2H-pyrrolyl, 1-pyrrolinyl, 2-pyrrolinyl, 3-pyrrolinyl, pyrrolidinyl, 4H-quinolizinyl, 4aH-carbazolyl, 2-oxopiperazinyl, piperazinyl, homopiperazinyl, 2-pyrazolinyl, 3-pyrazolinyl, pyranyl, dihydro-2H-pyranyl, 4H-pyranyl, 3,4-dihydro-2H-pyranyl, triazinyl, cinnolinyl, phthalazinyl, azepinyl, oxetanyl, thietanyl, 3-dioxolanyl, 1,4-dioxanyl, 2,5-dioximidazolidinyl, 2,2,4-piperidonyl, 2-oxopiperidinyl, 2-oxopyrrolidinyl, 2-oxoazepinyl, indolinyl, tetrahydropyranyl, tetrahydrofuranyl, tetrahydrothienyl, tetrahydroquinolinyl, tetrahydroisoquinolinyl, thiomorpholinyl, thiomorpholinyl sulfoxide, thiomorpholinyl sulfone, 1,3-dioxolanyl, 1,4-oxathianyl, 1,4-dithianyl, 1,3,5-trioxanyl, 6H-1,2,5-thiadiazinyl, 2H-1,5,2-dithiazinyl, 2H-oxocinyl, 1H-pyrrolizinyl, tetrahydro-1,1-dioxothienyl, N-formylpiperazinyl, 2,3-dihydrobenzo[1,4]dioxin-2-yl, 2,3-dihydrobenzo[1,4]dioxin-6-yl, and morpholinyl.

The term “aralkyl” by itself or as part of another substituent refers to a group having as alkyl moiety the aforementioned alkyl attached to one of the aforementioned aryl rings. Examples of aralkyl radicals include benzyl, phenethyl, dibenzylmethyl, methylphenylmethyl, 3-(2-naphthyl)-butyl, and the like.

The term “cycloalkylalkyl” by itself or as part of another substituent refers to a group having one of the aforementioned cycloalkyl groups attached to one of the aforementioned alkyl chains. Examples of such cycloalkylalkyl radicals include cyclopropylmethyl, cyclobutylmethyl, cyclopentylmethyl, cyclohexylmethyl, 1-cyclopentylethyl, 1-cyclohexylethyl, 2-cyclopentylethyl, 2-cyclohexylethyl, cyclobutylpropyl, cyclopentylpropyl, 3-cyclopentylbutyl, cyclohexylbutyl and the like.

The term “heterocyclyl-alkyl” by itself or as part of another substituents refers to a group having one of the aforementioned heterocyclyl group attached to one of the aforementioned alkyl group, i.e., to a group —R^{b}—R^{c} wherein R^{b} is alkylene or alkylene substituted by alkyl group and R^{c} is a heterocyclyl group.

The term “acyl” by itself or as part of another substituent refers to an alkanoyl group having 2 to 6 carbon atoms or a phenylalkanoyl group whose alkanoyl moiety has 1 to 4 carbon atoms, i.e; a carbonyl group linked to a radical such as, but not limited to, alkyl, aryl, more particularly, the group —COR¹¹, wherein R¹¹ can be selected from alkyl, aryl, substituted alkyl, or substituted aryl, as defined herein. The term acyl therefore encompasses the group alkylcarbonyl (—COR¹¹), wherein R¹¹ is alkyl. Said acyl can be exemplified by acetyl, propionyl, butyryl, valeryl and pivaloyl, benzoyl, phenylacetyl, phenylpropionyl and phenylbutylyl.

The term “alkylamino” by itself or as part of another substituent refers to a group consisting of an amino groups attached to one or two independently selected and optionally substituted alkyl groups, cycloalkyl groups, arylalkyl or cycloalkylalkyl groups i.e., —N(R⁶)(R⁷) wherein R⁶ and R⁷ are each independently selected from hydrogen, cycloalkyl, arylalkyl, cycloalkylalky or alkyl. Non-limiting examples of alkylamino groups include methylamino (NHCH₃), ethylamino (NHCH₂CH₃), n-propylamino, isopropylamino, n-butylamino, isobutylamino, sec-butylamino, tert-butylamino, n-hexylamino, and the like.

The term “keto” as used herein refers to the group ═O.

The term “amino” refers to the group —NH₂.

The term “aminocarbonyl” refers to the group —(C═O)—NH₂.

The term “aminoalkyl” refers to the group —R^{b}—NR^{d}R^{e} wherein R^{b} is alkylene or substituted alkylene, R^{d} is hydrogen or alkyl or substituted alkyl as defined herein, and R^{e} is hydrogen or alkyl as defined herein, wherein the substituents are the same as that described above for substituted alkyl.

The term “cyanoalkyl” refers to the group —R^{b}—CN wherein R^{b} is alkylene or substituted alkylene as defined herein, wherein the substituents are the same as that described above for substituted alkyl.

The term “alkylaminocarbonyl” refers to a group —(C═O)—NR^{d}R^{e} wherein R^{d} is hydrogen or alkyl or substituted alkyl as defined herein, and R^{e} is alkyl or substituted alkyl as defined herein, wherein the substituents are the same as that described above for substituted alkyl.

The term “alkylaminocarbonylamino” refers to a group —NH(C═O)—NR^{d}R^{e} or —NR′(C═O)—NR^{d}R^{e} wherein R^{d} is hydrogen or alkyl or substituted alkyl as defined herein, and R^{e} is alkyl or substituted alkyl as defined herein, wherein R′ is alkyl or substituted alkyl, wherein the substituents are the same as that described above for substituted alkyl.

The term “carboxy” refers to the group —CO₂H. Thus, a carboxyalkyl is an alkyl group as defined above having at least one substituent that is —CO₂H.

The term “alkoxycarbonyl” refers to a carboxy group linked to an alkyl radical i.e. to form —C(═O)OR¹¹, wherein R¹¹ is as defined above for acyl.

The term “alkylcarbonyloxy” refers to a —O—C(═O)R¹¹ wherein R¹¹ is as defined above for acyl.

The term “alkylamidyl” or “alkylamide” refers to an alkylcarbonylamino group of formula —NH(C═O)R or —NR′(C═O)R, wherein R and R′ are each independently alkyl or substituted alkyl, wherein the substituents are the same as that described above for substituted alkyl.

The term “alkylcarbonylaminoalkyl” refers to a group —R^{b}—NR^{d}—C(═O)—R^{e} wherein R^{b} is alkylene or alkylene substituted by alkyl, R^{d} is hydrogen or alkyl as defined herein, and R^{e} is alkyl as defined herein, wherein the substituents are the same as that described above for substituted alkyl.

The term “alkylamino(alkylsubstituted)alkyl” refers to a group —R^{f}NR^{d}R^{e} wherein R^{f} is alkylene substituted by alkyl, R^{d} is hydrogen or alkyl or substituted alkyl as defined herein, and R^{e} is alkyl or substituted alkyl as defined herein, wherein the substituents are the same as that described above for substituted alkyl.

The term “alkoxy” by itself or as part of another substituent refers to a group consisting of an oxygen atom attached to one straight or branched alkyl group, cycloalkyl group, arylalkyl or cycloalkylalkyl group, each group optionally substituted by one or more substitutents, wherein the substituents are the same as that described above for substituted alkyl. Non-limiting examples of suitable alkoxy group include methoxy, ethoxy, n-propoxy, isopropoxy, n-butoxy, iso-butoxy, sec-butoxy, tert-butoxy, hexanoxy and the like.

The term “alkylthio” by itself or as part of another substituent refers to a group consisting of a sulfur atom attached to one alkyl group, cycloalkyl group, arylalkyl or cycloalkylalkyl group, each optionally substituted by one or more substitutents, wherein the substituents are the same as that described above for substituted alkyl. Non-limiting examples of alkylthio groups include methylthio (SCH₃), ethylthio (SCH₂CH₃), n-propylthio, isopropylthio, n-butylthio, isobutylthio, sec-butylthio, tert-butylthio, n-hexylthio, and the like.

The term “acylamino” by itself or as part of another substituent refers to a group consisting of an amino group attached to one or two independently selected acyl groups as described before. In case the two acyl groups of a dicarboxylic acid are attached to the amino group these represent imides such as phtalimides, maleimides and the like, and are encompassed in the meaning of the term acylamino.

The term “halo” or “halogen” as a group or part of a group is generic for fluoro, chloro, bromo or iodo.

The term “haloalkyl” alone or in combination, refers to an alkyl radical having the meaning as defined above wherein one or more hydrogens are replaced with a halogen as defined above. Non-limiting examples of such haloalkyl radicals include chloromethyl, 1-bromoethyl, fluoromethyl, difluoromethyl, trifluoromethyl, 1,1,1-trifluoroethyl and the like.

The term “haloaryl” alone or in combination, refers to an aryl radical having the meaning as defined above wherein one or more hydrogens are replaced with a halogen as defined above.

The term “haloalkoxy” alone or in combination refers to a group of Formula —O-alkyl wherein the alkyl group is substituted by 1, 2 or 3 halogen atoms. For example, “haloalkoxy” includes —OCF₃ and —OCHF₂.

The term “sulphonamide” alone or in combination refers to a group of Formula —SO₂—NR^{d}R^{e} wherein R^{d} is hydrogen or alkyl or substituted alkyl as defined herein, and R^{e} is hydrogen or alkyl as defined herein, wherein the substituents are the same as that described above for substituted alkyl.

As used herein, the terms “optionally substituted alkyl, cycloalkyl, alkenyl or alkynyl” or “alkyl, cycloalkyl, alkenyl or alkynyl, optionally substituted” means that each group is optionally substituted i.e. “optionally substituted alkyl, optionally substituted cycloalkyl, optionally substituted alkenyl or optionally substituted alkynyl”, wherein the substituents are the same as that described above for substituted alkyl.

Whenever the term “substituted” is used in the present invention, it is meant to indicate that one or more hydrogens on the atom indicated in the expression using “substituted” is replaced with a selection from the indicated group, provided that the indicated atom's normal valency is not exceeded, and that the substitution results in a chemically stable compound, i.e. a compound that is sufficiently robust to survive isolation to a useful degree of purity from a reaction mixture, and formulation into a therapeutic agent.

Whenever used in the present invention the term “compounds of the invention” or a similar term is meant to include the compounds of general Formula I, II, III or IV and any subgroup thereof. This term also refers to the compounds as depicted in Table 12 and their derivatives, N-oxides, salts, solvates, hydrates, stereoisomeric forms, racemic mixtures, tautomeric forms, optical isomers, analogues, pro-drugs, esters and metabolites, as well as their quaternized nitrogen analogues. The N-oxide forms of said compounds are meant to comprise compounds wherein one or several nitrogen atoms are oxidized to the so-called N-oxide.

As used in the specification and the appended claims, the singular forms “a”, “an,” and “the” include plural referents unless the context clearly dictates otherwise. By way of example, “a compound” means one compound or more than one compound.

Asterisks (*) are used herein to indicate the point at which a mono-, bi- or trivalent radical depicted is connected to the structure to which it relates and of which the radical forms part.

The term “pro-drug” as used herein means the pharmacologically acceptable derivatives such as esters, amides and phosphates, such that the resulting in vivo biotransformation product of the derivative is the active drug. The reference by Goodman and Gilman (The Pharmacological Basis of Therapeutics, 8th Ed, McGraw-Hill, Int. Ed. 1992, “Biotransformation of Drugs”, p 13-15) describing pro-drugs generally is hereby incorporated. Pro-drugs of the compounds of the invention can be prepared by modifying functional groups present in said component in such a way that the modifications are cleaved, either in routine manipulation or in vivo, to the parent component. Typical examples of pro-drugs are described for instance in WO 99/33795, WO 99/33815, WO 99/33793 and WO 99/33792 all incorporated herein by reference. Pro-drugs are characterized by increased bio-availability and are readily metabolized into the active inhibitors in vivo.

In an embodiment, the present invention provides compounds of Formula I, II, III or IV wherein X¹ is —N(R³)—, in is 0 and wherein R³ has the same meaning as that defined hereinabove. In another embodiment, the present invention provides compounds of Formula I, II, III or IV wherein X¹ is 0 and n is 0. In yet another embodiment, the present invention provides compounds of Formula I, I, III or IV wherein X¹ is S and n is 0. In a further embodiment, the present invention provides compounds of Formula I, II, III or IV wherein X¹ is —N═ and X² is ═CH— and n is 1. In a further embodiment, the present invention provides compounds of Formula I, II, III or IV wherein Y is N and X¹ is —N(R¹³)—, wherein R¹³ is selected from hydrogen, alkyl, aralkyl or alkylcarbonyl.

According to a particular embodiment, the present invention provides compounds having the structural Formula V, VI, VII, VIII, IX or X 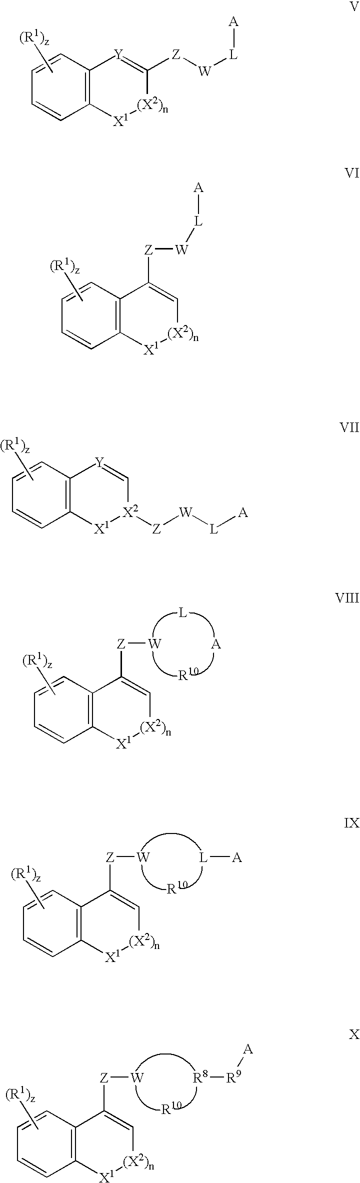
wherein R¹, z, X¹, X², W, Z, n, L, A, R⁸, R⁹, R¹⁰ and R² have the same meaning as that defined hereinabove.

According to another particular embodiment, the present invention provides compounds having the structural Formula XI, XII, XIII, XIV or XV 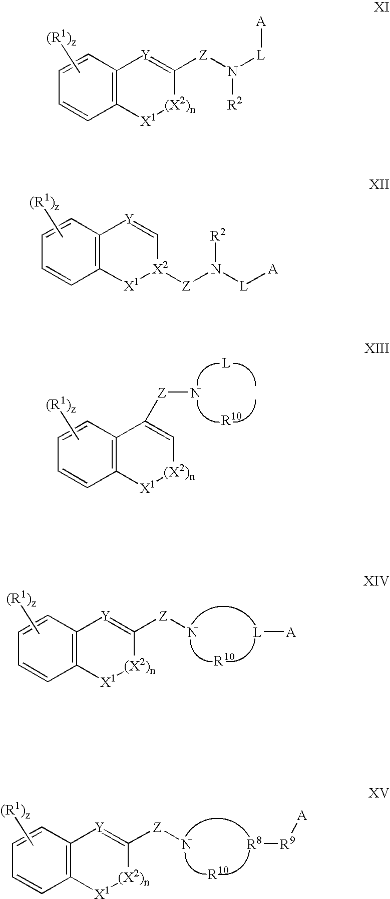
wherein R¹, z, X¹, X², Y, Z, n, L, A, R⁸, R⁹, R¹⁰ and R² have the same meaning as that defined hereinabove.

Preferably the present invention, provides compounds having the structural Formula XVI to XXXI 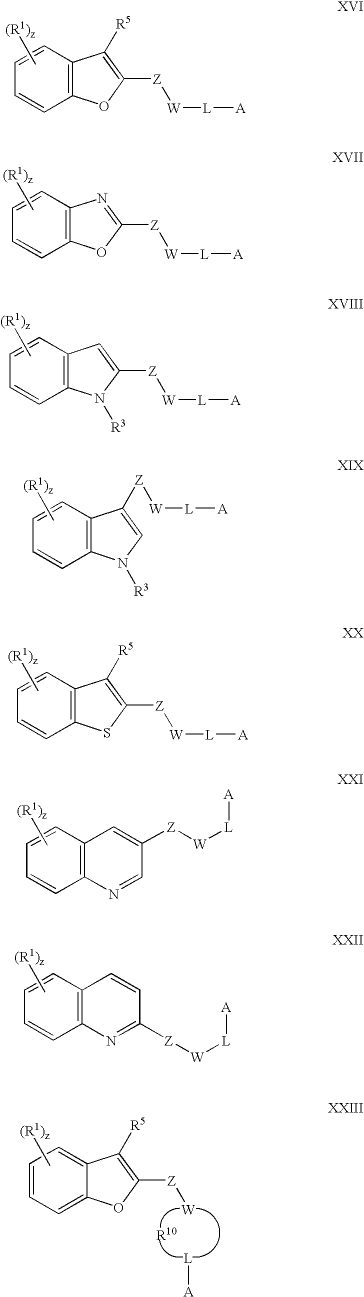 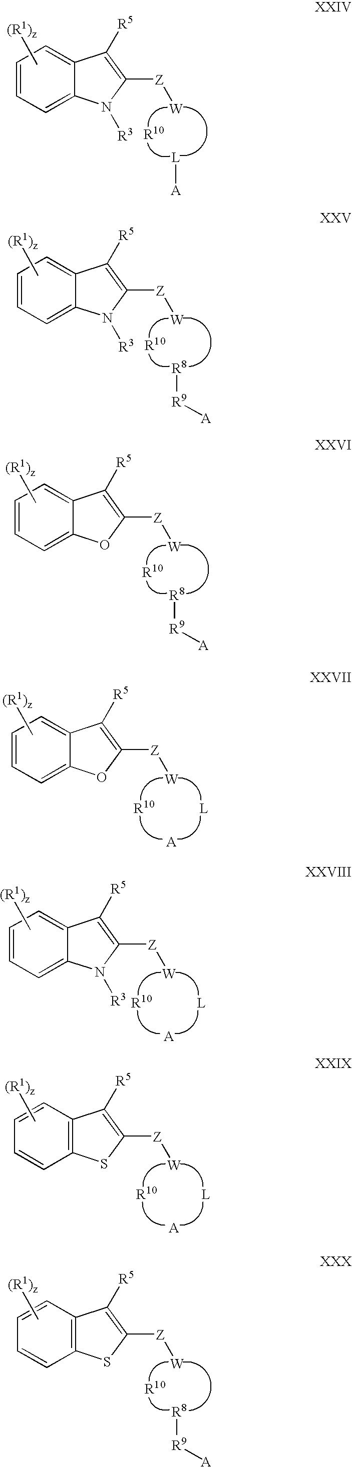 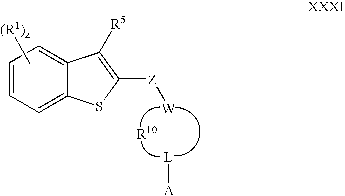
wherein R⁵ is selected from hydrogen, alkyl, or aralkyl and wherein R¹, z, Z, W, L, A, R⁸, R⁹, R¹⁰ and R³ have the same meaning as that defined hereinabove.

According to an embodiment, the present invention provides compounds of Formula XXXII to LXIII 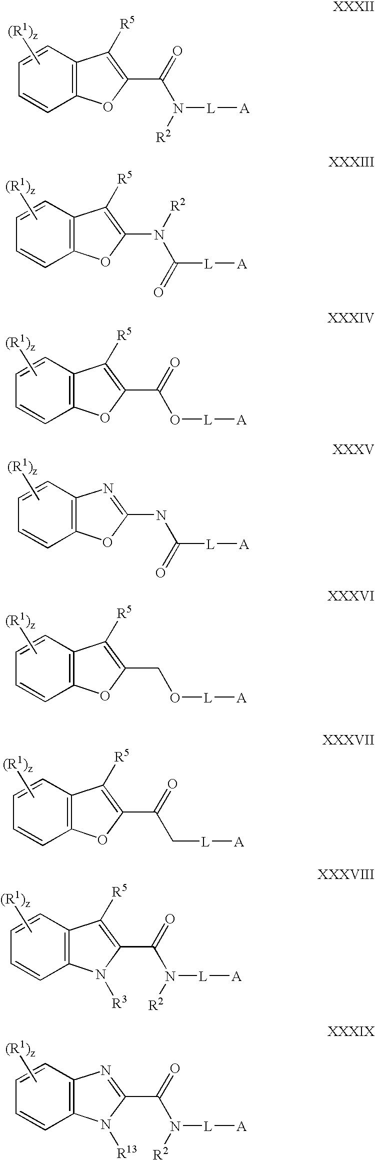 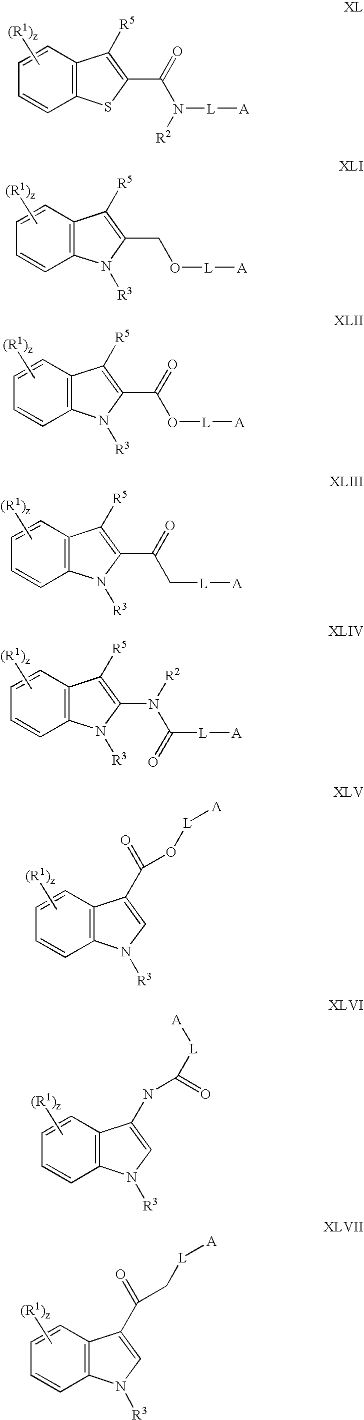 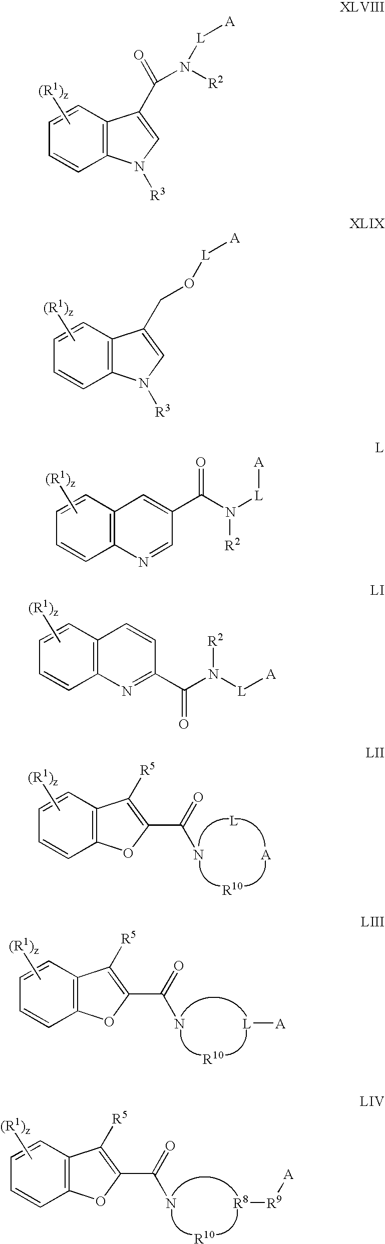 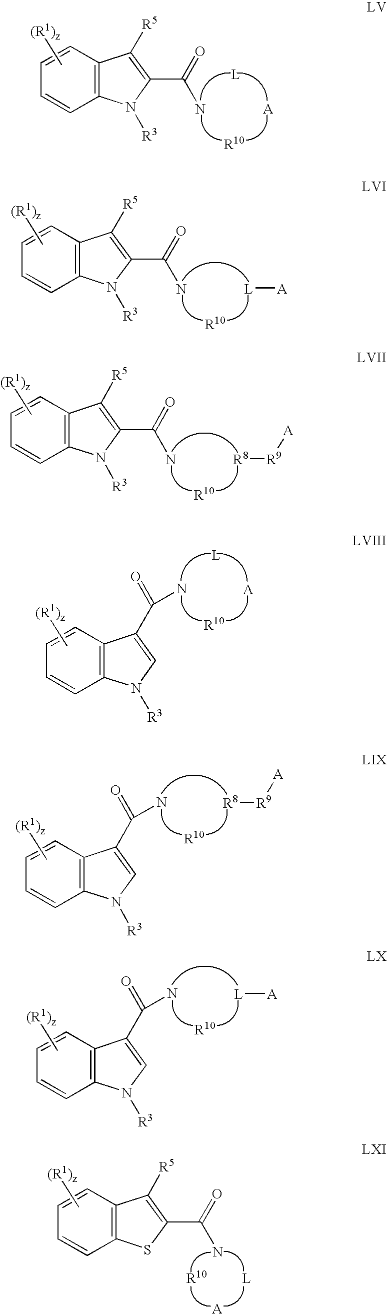 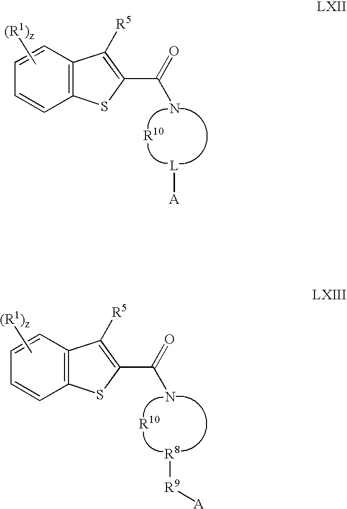
wherein R¹, z, R⁵, R², R³, R⁸, R⁹, R¹⁰, L and A have the same meaning as that defined herein above.

According to an embodiment, the present invention provides compounds of Formula I to LXIII, wherein A is selected from 2- or 3-furyl, 2- or 3-thienyl, 1-, 2- or 3-pyrrolyl, 1-, 2-, 4- or 5-imidazolyl, 1-, 3-, 4- or 5-pyrazolyl, 3-, 4- or 5-isoxazolyl, 2-, 4- or 5-oxazolyl, 3-, 4- or 5-isothiazolyl, 2-, 4- or 5-thiazolyl, 1,2,3-triazol-1-, -2-, -4- or -5-yl, 1,2,4-triazol-1-, -3-, -4- or -5-yl, 1,2,3-oxadiazol-4- or -5-yl, 1,2,4-oxadiazol-3- or -5-yl, 1,2,3-thiadiazol-4- or -5-yl, 1,2,4-thiadiazol-3- or -5-yl, 1,2,5-thiadiazol-3- or -4-yl, 1- or 5-tetrazolyl, phenyl, biphenyl, 2-, 3- or 4-pyridyl, pyridinyl, anthracenyl, azulenyl, indenyl, 3- or 4-pyridazinyl, 2-, 4-, 5- or 6-pyrimidinyl, 2-, 3-, 4-, 5- 6-2H-thiopyranyl, 2-, 3- or 4-4H-thiopyranyl, furyl, 2-, 3-, 4-, 5-, 6- or 7-benzofuryl, 2-, 3-, 4-, 5-, 6- or 7-benzothienyl, 1-, 2-, 3-, 4-, 5-, 6- or 7-indolyl, 1-, 2-, 4- or 5-benzimidazolyl, 1-, 3-, 4-, 5-, 6- or 7-benzopyrazolyl, 3-, 4-, 5-, 6- or 7-benzisoxazolyl, 2-, 4-, 5-, 6- or 7-benzoxazolyl, 3-, 4-, 5-, 6- or 7-benzisothiazolyl, 2-, 4-, 5-, 6- or 7-benzothiazolyl, 1,3-benzodioxolyl, 1- or 2-naphthyl, 2-, 3-, 4-, 5-, 6-, 7-, 8-quinolinyl, 2-, 4-, 5-, 6-, 7- or 8-quinazolyl, 1-, 3-, 4-, 5-, 6-, 7-, 8-isoquinolinyl, or 1-, 2-, 3-, 4- or 9-carbazolyl, 5,6,7,8-tetrahydronaphthyl, thienyl, benzothienyl, dibenzo[b,f]azepinyl, dibenzo[a,d]cylcoheptenyl, pyrrolyl, dioxanyl, thietanyl, oxazolyl, piperidinyl, imidazolinyl, isoxazolinyl, oxazolidinyl, thiazolidinyl, isothiazolidinyl, 2-oxopiperazinyl, pyrazinyl, triazinyl, cinnolinyl, phthalazinyl, oxetanyl, azepinyl, 4-piperidonyl, 2-oxopiperidinyl, 2-oxopyrrolodinyl, 5-(2,3-dihydro)benzofuranyl, 2-oxoazepinyl, tetrahydropyranyl, thiamorpholinyl, thiamorpholinyl sulfoxide, thiamorpholinyl sulfone, 1,3-dioxolane, tetrahydro-1,I-dioxothienyl, piperazinyl or morpholinyl, optionally substituted by 1, 2, 3 or 4 substituents selected from halogen, hydroxy, nitro, azido, hydrazino, cyano, alkyl, aryl, cycloalkyl, acyl, alkylamino, alkylaminocarbonyl, alkyloxycarbonyl, alkylcarbonyloxy, fused heterocyclyl, haloalkyl, alkylcarbonyl, heteroarylalkyl, aryloxy, arylcarbonyl, haloalkoxy, alkoxy, thiol, alkylthio, carboxy, acylamino, alkyl esters, carbamate, thioamide, urea, —SO₂R¹⁵, or sulphonamide, wherein R¹⁵ is alkylamino, alkyl or cycloalkyl, wherein L is selected from a single bond, a group of formula —R⁸—R⁹—, alkylyn, alkenylyl, cycloalkylene, —NH—(C(R⁴)(R⁴))_{q}—, —(C(R⁴)(R⁴))ᵥ—N—(C(R⁴)(R⁴))_{w}, —(C(R⁴)(R⁴))_{q}—, —(C(R⁴)(R⁴))ᵥ—O—(C(R⁴)(R⁴))_{w}—, —(C(R⁴)(R⁴))ᵥ—(C(R⁴))_{w}═, —(C(R⁴)(R⁴))_{q}—(C═O)—, or cycloalkylenoxyalkylene, wherein —(C(R⁴)(R⁴))_{q}—, (C(R⁴)(R⁴))_{w} and —(C(R⁴)(R⁴))ᵥ— are each independently aliphatic or form a cycloalkyl, wherein each R⁴ is independently selected from hydrogen, alkylaminoalkyl, hydroxyl, alkyl, carboxy, hydroxyalkyl, alkoxyalkyl, alkylamino, or alkyloxycarbonyl; q is an integer selected from 0, 1, 2, 3, or 4; v is an integer selected from 0, 1, 2, 3 or 4 and w is an integer selected from 0, 1, 2, 3 or 4, and wherein R⁸ is alkylyn, —(C(R⁴)(R⁴))ₚ—C(R¹⁴) or —(C(R⁴)(R⁴))ₚ—C(R⁴)═C, wherein R⁹ is a single bond, —(C(R⁴)(R⁴))_{q}—, or —C(═O)—, wherein R¹⁴ is selected from hydrogen, hydroxyl or alkyl, wherein p is an integer selected from 0, 1, 2 or 3, and wherein R¹⁰ is selected from —(C(R⁴)(R⁴))ₘ—, —(C(R⁴)(R⁴))ₘ—C(═O)O—(C(R⁴)(R⁴))_{q}—, or —(C(R⁴)(R⁴))ₘ—N(R¹²)—(C(R⁴)(R⁴))_{q}—, wherein m is an integer selected from 1, 2, 3, 4, 5 or 6, wherein R¹² is selected from hydrogen, alkyl, aryl, arylalkyl, or alkylcarbonyl and wherein R² is selected from hydrogen, CH₃—, —CH₂—CH₃, —(CH₂)₂—CH₃, —(CH₂)₂—CN, phenyl, benzyl or CH₃—C(═O).

According to another embodiment, the present invention provides compounds having a structural formula selected from Formula XVI to XXII and XXXII to LI, wherein A is selected from phenyl, 3-indolyl, 5-(2,3-dihydro)benzofuranyl, 6-indolyl, 4-pyridinyl, 1,3-benzodioxolyl, 2-thienyl, 2-naphthyl, dibenzo[b,f]azepinyl, or dibenzo[a,d]cylcoheptenyl, each optionally substituted by 1; 2, 3 or 4 substituents selected from —OCH₃, —NO₂, —CO₂H, —C(═O)—N(CH₃)₂, —O—C(═O)—CH₃, —CH₃, —CH₂—CH₃, phenyl, —SO₂—CH₃, F, Cl, Br, —CF₃, —S—CH₃, —OCF₃, —C(═O)—CH₃, —O—C(═O)—CH₃, —C(═O)O—CH₃, —C(═O)N(CH₃)₂, —N(CH₃)₂, —SO₂—N(CH₃)₂, L), 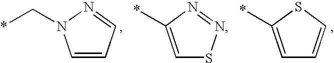
N-morpholino, phenoxyl, benzoyl, —C(CH₃)₃, —O—(CH₂)₂—CH₃, —OH or —CN,
wherein L is selected from single bond, —CH₂—, —(CH₂)₂—, —(CH₂)₃—, —CH(CH₂OH)—, —CH(CH₂—O—CH₃)—, —CH(CH₃)—, —CH(CH₂—CH₃)—, —CH(CO₂H)—, —CH(CO₂CH₃)—, —(CH₂)₂—O—CH₂—, —CH(CH₂—N(CH₃)₂)—, —(CH₂)₂—CH═, or 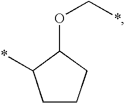
or wherein -L-A is 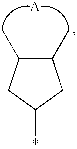
or 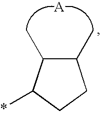
wherein R¹ is selected from hydrogen, Cl, Br, F, —CF₃, —OCH₃, CH₃—C(═O)—, (CH₃)₂N—CH(CH₃)—, —CO₂H, (CH₃)₂N—C(═O)—, 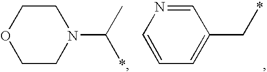
CH₃—C(═O)O—, CH₃—O—C(═O)—, CH₃—NH—C(═O)—, CH₃—C(═O)—NH—CH(CH₃)—, HO—CH₂—, CH₃—CH₂—, CH₃—O—CH₂—, wherein z is 1 or 2, wherein R⁵ is selected from H, —NH₂, CH₃—NH—C(═O)—NH—, CH₃—C(═O)—NH—, CH₃—C(═O)O—, —OH, —CH₃, CH₃—CH₂—, (CH₃)₂N—, or 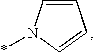
and wherein R² is selected from hydrogen, CH₃—, phenyl, CH₃—, —CH₂—CH₃, —(CH₂)₂—CH₃, —(CH₂)₂—CN, benzyl or CH₃—C(═O).

According to another embodiment, the present invention provides compounds having a structural formula selected from Formula XXIII to XXXI and LII to LXIII, wherein the group 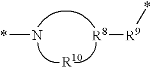
is selected from 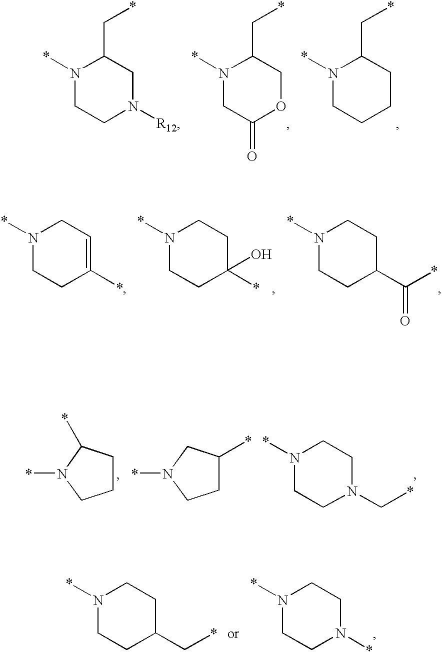
wherein the group 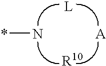
is selected from 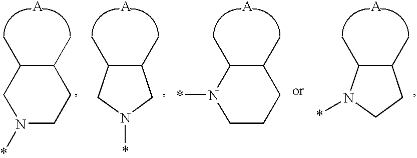
wherein the group 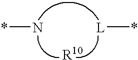
is selected from 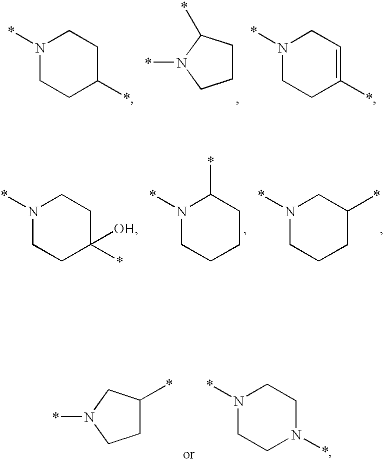
wherein A is selected from phenyl, 3-indolyl, 5-(2,3-dihydro)benzofuranyl, 6-indolyl, 4-pyridinyl, 2-thienyl, 2-naphthyl, 1,3-benzodioxolyl, dibenzo[b,f]azepinyl, dibenzo[a,d]cylcoheptenyl, each optionally substituted by 1, 2, 3 or 4 substituents selected —OCH₃, —NO₂, —CO₂H, —C(═O)—N(CH₃)₂, —O—C(═O)—CH₃, —CH₃, —CH₂—CH₃, phenyl, —SO₂—CH₃, F, Cl, Br, —CF₃, —S—CH₃, —OCF₃, —C(═O)—CH₃, —O—C(═O)—CH₃, —C(═O)O—CH₃, —C(═O)N(CH₃)₂, —N(CH₃)₂, —SO₂—N(CH₃)₂, 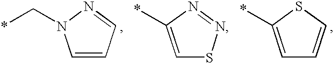
N-morpholino, phenoxyl, benzoyl, —C(CH₃)₃, —O—(CH₂)₂—CH₃, —OH or —CN, wherein R¹² is selected from hydrogen, CH₃—C(═O)—, CH₃— or benzyl, and wherein R¹ is selected from hydrogen, Cl, Br, F, —CF₃, —OCH₃, CH₃—C(═O)—, —CO₂H, (CH₃)₂N—C(═O)—, 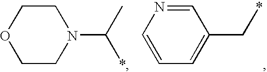
CH₃—C(═O)O—, CH₃—O—C(═O)—, CH₃—NH—C(═O)—, CH₃—C(═O)—NH—CH(CH₃)—, HO—CH₂—, CH₃—CH₂—, CH₃—O—CH₂—, wherein z is 1 or 2, wherein R⁵ is selected from H, CH₃—NH—C(═O)—NH—, CH₃—C(═O)—NH—, CH₃—C(═O)O—, —OH, —CH₃, CH₃—CH₂—, (CH₃)₂N—, or 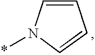
and wherein R² is selected from hydrogen, CH₃—, CH₃—, —CH₂—CH₃, —(CH₂)₂—CH₃, —(CH₂)₂—CN, phenyl, benzyl or CH₃—C(═O).

In a particular embodiment, the present invention provides compounds as described herein wherein R⁵ is selected from hydrogen, CH₃—, —NH₂, CH₃—C(═O)—NH—, or 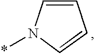
wherein R² is selected from hydrogen, CH₃—, phenyl, CH₃—, —CH₂—CH₃, —(CH₂)₂—CH₃, or —(CH₂)₂—CN,
wherein R¹ is selected from H, Cl, F, Br, —OCH₃, —C(═O)CH₃, wherein z is an integer selected from 1, 2 or 3,
wherein A is selected from phenyl, 3-indolyl, 5-(2,3-dihydro)benzofuranyl, 6-indolyl, 4-pyridinyl, 2-thienyl, 1,3-benzodioxolyl, 2-naphthyl, dibenzo[b,f]azepinyl, dibenzo[a,d]cylcoheptenyl, each optionally substituted by 1; 2 or 3 substituents selected from —OCH₃, —NO₂, —CO₂H, —C(═O)—N(CH₃)₂, —O—C(═O)—CH₃, —CH₃, —CH₂—CH₃, phenyl, —SO₂—CH₃, F, Cl, Br, —CF₃, —S—CH₃, —OCF₃, —C(═O)—CH₃, —O—C(═O)—CH₃, —C(═O)O—CH₃, —C(═O)N(CH₃)₂, —N(CH₃)₂, —SO₂—N(CH₃)₂, 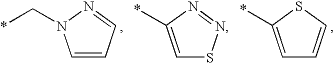
N-morpholino, phenoxyl, benzoyl, —C(CH₃)₃, —O—(CH₂)₂—CH₃, —OH or —CN,
wherein L is a linking group selected from a single bond, a group of formula —R⁸—R⁹—, alkylyn, alkenylyl, cycloalkylene, —NH—(C(R⁴)(R⁴))_{q}—, —(C(R⁴)(R⁴))_{q}—, —(C(R⁴)(R⁴))ᵥ—O—(C(R⁴)(R⁴))_{w}—, —(C(R⁴)(R⁴))ᵥ—N—(C(R⁴)(R⁴))_{w}—, —(C(R⁴)(R⁴))ᵥ—(C(R⁴))_{w}═, —(C(R⁴)(R⁴))_{q}—(C═O)—, or cycloalkylenoxyalkylene, wherein —(C(R⁴)(R⁴))_{q}—, (C(R⁴)(R⁴))_{w} and —(C(R⁴)(R⁴))ᵥ— are each independently aliphatic or form a cycloalkyl, wherein each R⁴ is independently selected from hydrogen, hydroxyl, alkyl, carboxy, hydroxyalkyl, alkoxyalkyl, alkylamino, alkylaminoalkyl or alkyloxycarbonyl; q is an integer selected from 0, 1, 2, 3, 4, 5 or 6; v is an integer selected from 0, 1, 2, 3, 4, 5 or 6 and w is an integer selected from 0, 1, 2, 3, 4, 5 or 6,
wherein R⁸ is alkylyn, —(C(R⁴)(R⁴))ₚ—C(R¹⁴) or —(C(R⁴)(R⁴))ₚ—C(R⁴)═C, wherein R⁹ is selected from a single bond, —(C(R⁴)(R⁴))_{q}—, or —C(═O)—, wherein R¹⁴ is selected from hydrogen, hydroxyl or alkyl, wherein p is an integer selected from 0, 1, 2 or 3 and
wherein R¹⁰ is selected from —(C(R⁴)(R⁴))ₘ—, —(C(R⁴)(R⁴))ₘ—C(═O)O—(C(R⁴)(R⁴))_{q}—, or —(C(R⁴)(R⁴))ₘ—N(R¹²)—(C(R⁴)(R⁴))_{q}—, wherein m is an integer selected from 1, 2, 3, 4, 5 or 6,
wherein R¹² is selected from hydrogen, alkyl, aryl, arylalkyl, or alkylcarbonyl. The present invention encompasses all the compounds having a Formula selected from the group comprising compounds of Formula I to LXIII as shown hereunder, wherein R¹, z, X¹, X², W, Y, Z, n, L, A, R², R³, R⁸, R⁹ and R¹⁰ have the same meaning as that defined above.

According to an embodiment, the present invention provides compounds of Formula I to LXIII, X¹ is a heteroatom selected from —O—, —S—, —N═, or —N(R³)—, wherein R³ is selected from alkyl, aralkyl or alkylcarbonyl, wherein X² is selected from C, ═CH— or —CH₂—, wherein n is an integer selected from 0 or 1, wherein Y is selected from C, —C(R⁵)— or N, wherein R⁵ is selected from hydrogen, amino, alkyl, hydroxyl, alkylamino, heteroaryl, alkylcarbonyloxy, alkylamidyl, or alkylaminocarbonylamino, wherein Z is selected from —C(═O)—, —CH₂—, or —NH—,

wherein W is selected from —C(═O)—, —N(R²)—, —N(R²)—NH—, —C(═O)—NH—, —CH═, —O— or —CH₂— in formula I and subformula thereof, and W is selected from N, or CH in formula II, III or IV and subformula thereof,

wherein A is selected from phenyl, 3-indolyl, 5-(2,3-dihydro)benzofuranyl, 6-indolyl, 4-pyridinyl, 1,3-benzodioxolyl, 2-thienyl, 2-naphthyl, dibenzo[b,f]azepinyl, or dibenzo[a,d]cylcoheptenyl, each optionally substituted by 1; 2, 3 or 4 substituents selected from —OCH₃, —NO₂, —CO₂H, —C(═O)—N(CH₃)₂, —O—C(═O)—CH₃, —CH₃, —CH₂—CH₃, phenyl, —SO₂—CH₃, F, Cl, Br, —CF₃, —S—CH₃, —OCF₃, —C(═O)—CH₃, —O—C(═O)—CH₃, —C(═O)O—CH₃, —C(═O)N(CH₃)₂, —N(CH₃)₂, —SO₂—N(CH₃)₂, 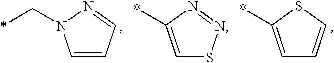
N-morpholino, phenoxyl, benzoyl, —C(CH₃)₃, —O—(CH₂)₂—CH₃, —OH or —CN,
wherein L is selected from single bond, —CH₂—, —(CH₂)₂—, —(CH₂)₃—, —CH(CH₂OH)—, —CH(CH₂—O—CH₃)—, —CH(CH₃)—, —CH(CH₂—CH₃)—, —CH(CO₂H)—, —CH(CO₂CH₃)—, —(CH₂)₂—O—CH₂—, —CH(CH₂—N(CH₃)₂)—, —(CH₂)₂—CH═, or 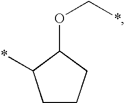
or wherein -L-A is 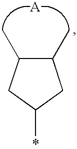
or 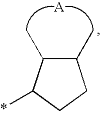
or wherein the group 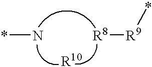
is selected from 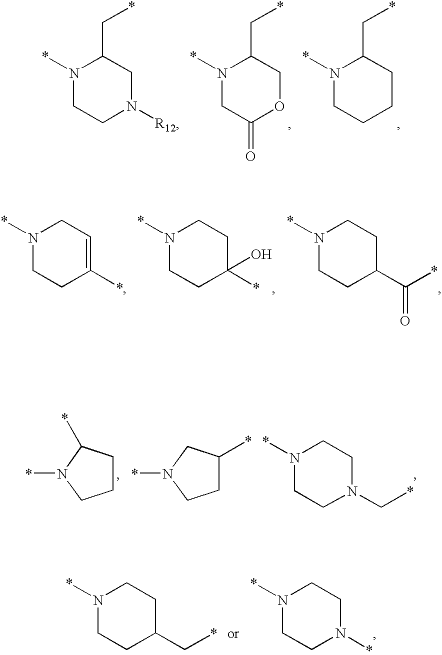
wherein the group 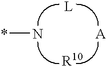
is selected from 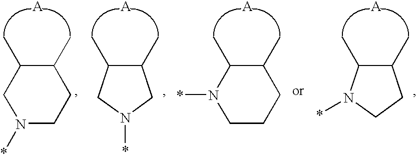
wherein the group 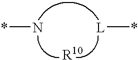
is selected from 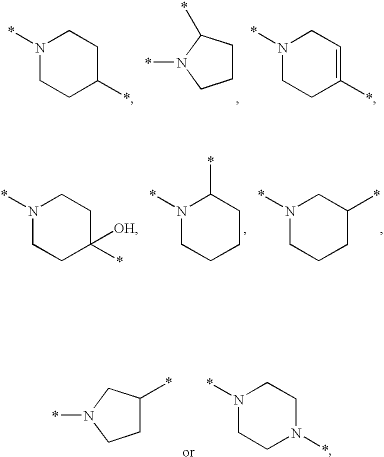
wherein R¹ is selected from hydrogen, Cl, Br, F, —CF₃, —OCH₃, CH₃—C(═O)—, (CH₃)₂N—CH(CH₃)—, —CO₂H, (CH₃)₂N—C(═O)—, 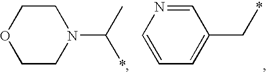
CH₃—C(═O)O—, CH₃—O—C(═O)—, CH₃—NH—C(—O)—, CH₃—C(═O)—NH—CH(CH₃)—, HO—CH₂—, CH₃—CH₂—, CH₃—O—CH₂—,
wherein z is 1 or 2,
wherein R⁵ is selected from H, —NH₂, CH₃—NH—C(═O)—NH—, CH₃—C(═O)—NH—, CH₃—C(═O)O—, —OH, —CH₃, CH₃—CH₂—, (CH₃)₂N—, or 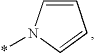
and
wherein R² is selected from hydrogen, CH₃—, phenyl, CH₃—, —CH₂—CH₃, —(CH₂)₂—CH₃, —(CH₂)₂—CN, benzyl or CH₃—C(—O).

According to a particular embodiment, the invention provides compound of formula XXXII, XXXIII, XXXIV, XXXV, XXXVI, XXXVII, XXXVIII, XXXIX, XL, XLI, XLII, XLIII, XLIV, XLV, XLVI, XLVII, XLVIII, XLIX, L, or LI
wherein A is selected from phenyl, 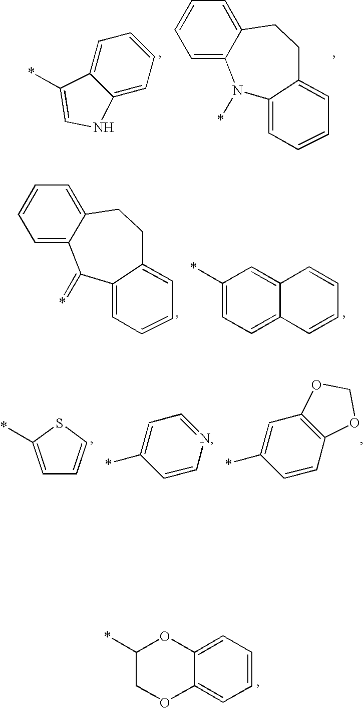
each optionally substituted by 1; 2, or substituents selected from —OCH₃, —NO₂, —CH₃, Cl, Br, —N(CH₃)₂, —O—C(═O)—CH₃, 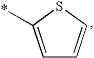
F, —CF₃, —S—CH₃, —C(═O)O—CH₃, —C(═O)N(CH₃)₂, —SO₂—N(CH₃)₂, phenoxyl, —C(CH₃)₃, phenyl, —C(═O)—CH₃, —SO₂—CH₃, —CN, —OCF₃, 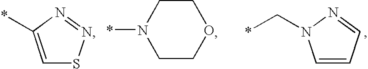
or —OH,
wherein L is selected from single bond, —CH₂—, —(CH₂)₂—, —(CH₂)₃—, —CH(CH₂OH)—, —CH(CH₂—O—CH₃)—, —CH(CH₃)—, —CH(CH₂—CH₃)—, —CH(CO₂H)—, —CH(CO₂CH₃)—, —(CH₂)₂—O—CH₂—, —CH(CH₂—N(CH₃)₂)—, —(CH₂)₂—CH═, or 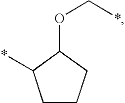
or wherein -L-A is 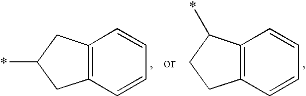
wherein A is optionally substituted with 1; 2, or 3 substituents selected from —OCH₃, —NO₂, —CH₃, Cl, Br, —N(CH₃)₂, —O—C(═O)—CH₃, 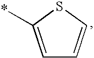
F, —CF₃, —S—CH₃, —C(═O)O—CH₃, —C(═O)N(CH₃)₂, —SO₂—N(CH₃)₂, phenoxyl, —C(CH₃)₃, phenyl, —C(═O)—CH₃, —SO₂—CH₃, —CN, —OCF₃, 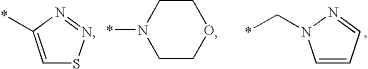
or —O H,
wherein R¹ is selected from H, Cl, Br, F, —CF₃, —OCH₃, CH₃—C(═O)—,
wherein z is 1 or 2,
wherein R⁵ is selected from H, —CH₃, 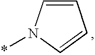
—NH₂, CH₃—C(═O)—NH—, or, CH₃—NH—C(═O)—NH—, and
wherein R² is selected from H, CH₃—, —(CH₂)₂—CH₃, phenyl, CH₃—, —CH₂—CH₃, —(CH₂)₂—CN, benzyl or CH₃—C(═O).

According to a particular embodiment, the invention provides compound of formula LII, LIII, LIV LV, LVI, LVII, LVIII, LIX, LX, LXI, LXII, or LXIII
wherein the group 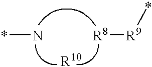
is selected from 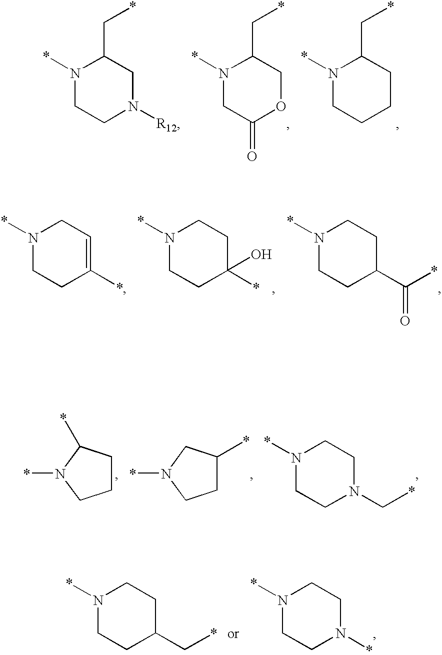
wherein the group 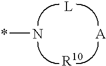
is selected from 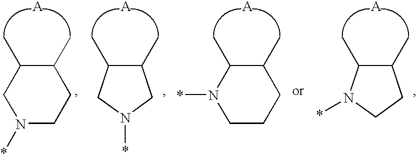
wherein the group 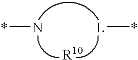
is selected from 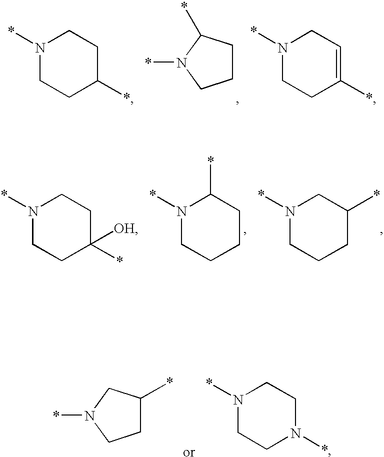
wherein A is selected from phenyl, 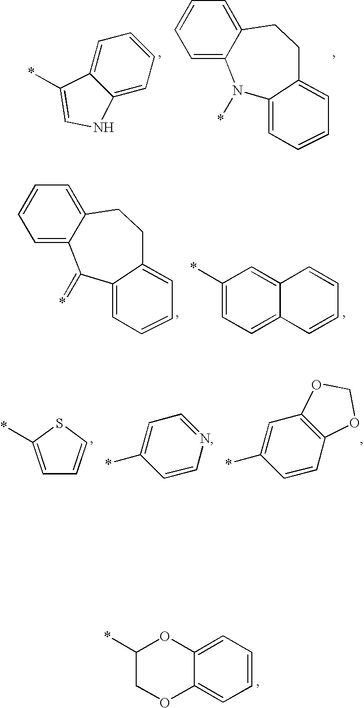
each optionally substituted by 1; 2, or substituents selected from —OCH₃, —NO₂, —CH₃, Cl, Br, —N(CH₃)₂, —O—C(═O)—CH₃, 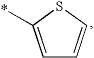
F, —CF₃, —S—CH₃, —C(═O)O—CH₃, —C(═O)N(CH₃)₂, —SO₂—N(CH₃)₂, phenoxyl, —C(CH₃)₃, phenyl, —C(═O)—CH₃, —SO₂—CH₃, —CN, —OCF₃, 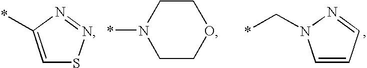
or —OH,
wherein R¹ is selected from H, Cl, Br, F, —CF₃, —OCH₃, CH₃—C(═O)—,
wherein z is 1 or 2,
wherein R⁵ is selected from H, —CH₃, 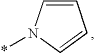
—NH₂, CH₃—C(═O)—NH—, or, CH₃—NH—C(═O)—NH—, and
wherein R² is selected from H, CH₃—, —(CH₂)₂—CH₃, phenyl, CH₃—, —CH₂—CH₃, —(CH₂)₂—CN, benzyl or CH₃—C(═O).

According to a particular embodiment of the present invention, the compounds have structural formula LXIX, 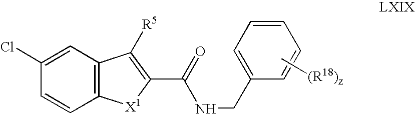
wherein X¹ is selected from O, S or —N—CH₃, wherein R⁵ is selected from hydrogen, methyl, or pyrryl, wherein R¹⁸ is selected from —OCH₃, F, Cl, Me, —OCF₃ or —CF₃ and wherein z is an integer selected from 1 or 2. In a particular embodiment, X¹ is O or S, z is 2 and R¹⁸ is —OCH₃, and R⁵ is H or CH₃.

According to a particular embodiment of the present invention, the compounds have structural formula LXX, 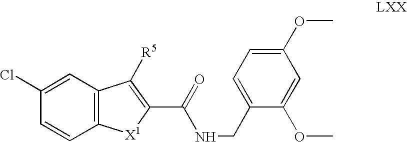
wherein X¹ is selected from O, S or —N—CH₃, wherein R⁵ is selected from hydrogen, methyl, or pyrryl. In a particular embodiment, X¹ is O or S, preferably S, and R⁵ is H or —CH₃ preferably —CH₃.

The present invention also relates to methods for the preparation of the compounds according to the present invention, using for example structurally related compounds. In an embodiment of the present invention, the compounds of the present invention can be prepared using the non-limiting methods described hereunder and in the examples.

In an embodiment, the method comprises the step of condensing a compound of Formula LXIV 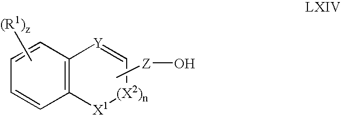
with a compound of Formula LXV, LXVI, LXVII or LXVIII: 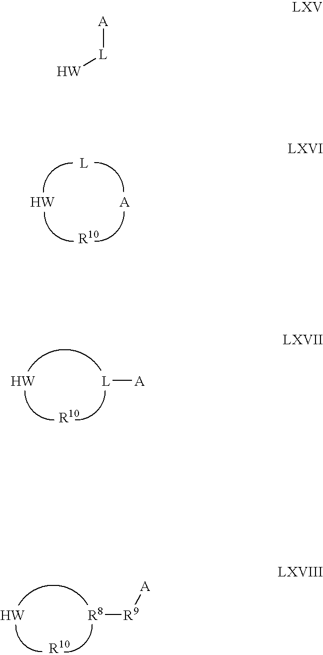
thereby obtaining a compound of Formula I, II, III or IV 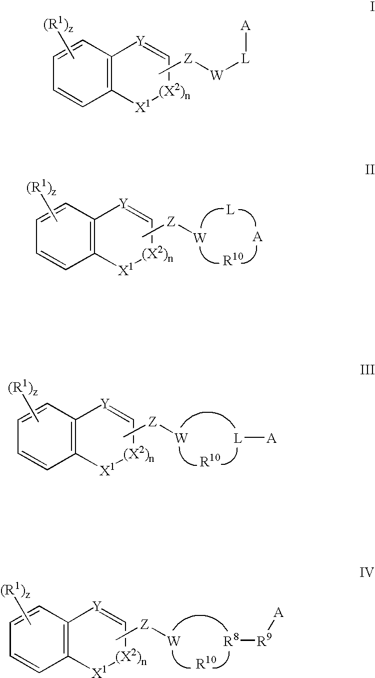
wherein R¹, z, X¹, X², W, Y, Z, n, L, A, R⁸, R⁹ and R¹⁰ have the same meaning as that defined hereinabove.

For example, the condensation can be performed via the formation of the acyl chloride of the compound of Formula LXIV and then by the coupling of said acyl chloride with a compound of Formula LXV, LXVI, LXVII or LXVIII, wherein W is N. In another embodiment, the condensation can be performed by using a suitable coupling agent, in a suitable solvent, in the presence of suitable base.

In a particular embodiment, the method for preparing compounds of the invention comprises the step of condensation of for example an acid of Formula LXIIIa: 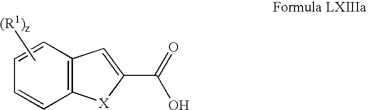
wherein R¹, and z have the meanings indicated hereinabove and X is X¹ as defined hereinabove, with an amine of Formula LXIVa, LXVa, LXVIa, or LXVIIa: 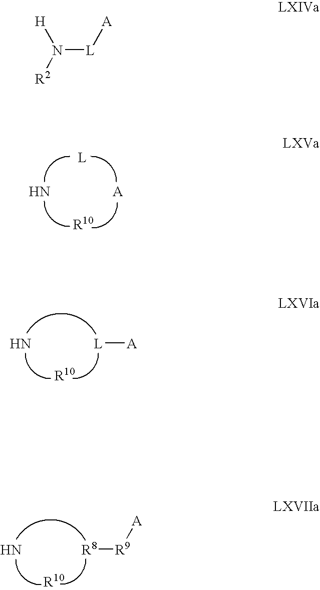
wherein A, L, R¹⁰ and R² have the meanings indicated hereinabove.

The reaction can generally be performed by condensing the compound of formula LXIIIa with a compound of formula LXIVa, LXVa, LXVIa, or LXVIIa.

The condensation can be performed via the formation of the acyl chloride of the acid of Formula LXIIIa and then by the coupling of said acyl chloride with the amine of Formula LXIVa, LXVa, LXVIa, or LXVIIa. In another embodiment, the condensation can be performed by using a suitable coupling agent, in a suitable solvent, in the presence of suitable base. The suitable coupling agent can be selected from the group comprising dicyclo-hexylcarbodiimide, hydroxybenzotriazole, o-benzotriazol-1-yl-N,N,N′,N4-tetramethyluronium hexafluorophosphate and the like and mixture thereof. The suitable solvent can be selected from the group comprising dichloromethane, dimethylformamide and the like or mixture thereof. Non limiting examples of suitable base comprise potassium carbonate, diisopropylethylamine, triethylamine, triisopropylamine and the like.

As described above, the condensation can be realized via formation of the corresponding acyl chloride and then coupling with the desired amine. In another embodiment the condensation can be performed using a suitable coupling agent, such as hydroxybenzotriazole (HOBT), o-benzotriazol-1-yl-N,N,N′,N-4-tetramethyluronium hexafluorophosphate (TBTU) and the like at a suitable molar ratio, for example between 1:1 to 1:3 relative to the acid derivative; in a suitable solvent or solvent mixture, such as dichloromethane (DCM) or dimethylformamide (DMF) and the like; at a suitable temperature, usually between 0° C. and the boiling point of the solvent used; for a suitable period of time, usually between 0.25 hr and 48 hrs; in the presence of a suitable base, for example an organic base such as potassium carbonate (K₂CO₃), diisopropylethylamine (DIEA), triethylamine (TEA), triisopropylamine and the like, in an amount between 0.1 and 5.0 equivalents.

The starting material for this reaction is either commercially available or can be prepared in a manner known per se.

The compounds of the present invention may then be isolated from the reaction mixture and may optionally be further purified, using techniques known per se, such as evaporation of the solvent, washing, trituration, recrystallisation from a suitable solvent or solvent mixture, and chromatographic techniques, such as column chromatography—for example using silica gel or C18 as solid phase- or preparative thin layer chromatography.

The term “stereoisomer” as used herein, defines all possible compounds made up of the same atoms bonded by the same sequence of bonds but having different three-dimensional structures which are not interchangeable, which the compounds of the present invention may possess. It will be clear to the skilled person that some of the compounds of the invention may contain one or more asymmetric carbon atoms that serve as a chiral center, which may lead to different optical forms (e.g. enantiomers or diastereoisomers). Unless otherwise mentioned or indicated, the chemical designation of a compound herein encompasses all such optical forms in all possible configurations as well as the mixture of all possible stereochemically isomeric forms, which said compound may possess. Said mixture may contain all diastereomers and/or enantiomers of the basic molecular structure of said compound. All stereochemically isomeric forms of the compounds of the invention either in pure form or in a mixture with each other are intended to fall within the scope of the present invention.

More generally, from the above, it will be clear to the skilled person that some of the compounds of the invention may exist in the form of different isomers and/or tautomers, including but not limited to geometrical isomers, conformational isomers, and stereochemical isomers (i.e. enantiomers and diastereoisomers) and isomers that correspond to the presence of the same substituents on different positions of the rings present in the compounds of the invention. All such possible isomers, tautomers and mixtures thereof are included within the scope of the invention.

It will also be clear that when the desired compounds of the invention, and/or the starting materials, precursors and/or intermediates used in the preparation thereof, contain functional groups that are sensitive to the reaction conditions used in the preparation of the compounds of the invention (i.e. that would undergo undesired reactions under those conditions if they were not suitably protected) can be protected during said reaction with one or more suitable protective group, which protective group can then be suitably removed after either completion of said reaction and/or as a later or final step in the preparation of the compounds of the invention. Protected forms of the inventive compounds are included within the scope of the present invention. Suitable protective groups, as well as methods and conditions for inserting them and removing them, will be clear to the skilled person and are generally described in the standard handbooks of organic chemistry, such as Greene and Wuts, “Protective groups in organic synthesis”, 3^{rd} Edition, Wiley and Sons, 1999, which is incorporated herein by reference in its entirety. It will also be clear to the skilled person that compounds of the invention in which one or more functional groups have been protected with suitable functional groups can find use as intermediates in the production and/or synthesis of the compounds of the invention, and as such form a further aspect of the invention.

The present invention further encompasses compounds obtainable by the methods according to the invention.

It was surprisingly found that the compounds of the invention interact with ion channels as shown in the examples below, in particular with ion channels from the Kv family, more in particular with ion channels from the Kv4 subfamily, and especially with Kv4.3 channels.

By “interact with” is meant that the compounds of the invention act as antagonists of said ion channel(s) and/or of the biological function(s) and/or pathways associated with these channels, and in particular that the compounds of the invention can fully or partially “block” said channels. Preferably, the compounds of the invention interact with ion channels from an animal, preferably a vertebrate animal, more preferably a warm-blooded animal, even more preferably a mammal, and most preferably a human being.

In an embodiment of the present invention, the compounds of the invention act as antagonists of said ion channels and/or of the biological functions or pathways associated therewith. Preferably, the compounds of the invention block said ion channels.

In a further embodiment, the compounds of the invention act as antagonists of ion channels from the Kv family and/or of the biological functions or pathways associated therewith. Also, preferably, the compounds of the invention block ion channels from the Kv family. In the invention, particular preference is given to compounds of Formula I, II, III or IV above that are particularly active against Kv4.3 ion channels and Kv1.5 ions channels and exhibit an IC₅₀ value of less than 100 μM, preferably less than 50 μM, more preferably less than 10 μM, preferably less than 5 μM, even more preferably less than 1 preferably less than 0.1 μM, and in particular less than 10 nM, less than 1 nM, as determined by a suitable assay, such as the assay used in the Examples below.

In the invention, particular preference is given to compounds of Formula I, II, III or IV above that are particularly active against Kv4.3 ion channels and Kv1.5 ions channels and wherein the remaining current measured after application of the compound and relative to the blank is equal or less than 90%, preferably less than 85%, more preferably less than 80%, preferably less than 70% as determined by a suitable assay, such as the assay used in the Examples below.

In a yet further embodiment, the compounds of the invention act as antagonists of ion channels from the Kv4 subfamily and/or of the biological functions or pathways associated therewith. Also, preferably, the compounds of the invention block ion channels from the Kv4 sub family.

According to a yet further embodiment, the compounds of the invention act as antagonists of the Kv4.3 ion channel and/or of the biological functions or pathways associated therewith. Also, most preferably, the compounds of the invention block the Kv4.3 ion channel.

According to a further aspect, the compounds of the invention which block the Kv4.3 ion channels, also block ion channels of the Kv1 subfamily, especially the Kv1.5 ion channel.

Whether a compound of the invention interacts with an ion channel can be determined using a suitable technique or assay, such as the assays described in the examples.

The compounds of the invention can therefore generally be used (1) as antagonists of ion channels and/or of the biological functions or pathways associated therewith, i.e. in an in vitro, in vivo or therapeutic setting; (2) as blockers of ion channels, i.e. in an in vitro, in vivo or therapeutic setting; and/or (3) as pharmaceutically active agents, in particular in (the preparation of pharmaceutical compositions for) the prevention and/or treatment of conditions or diseases associated with said ion channels.

In particular, the compounds of the invention that interact with ion channels from the Kv family can be used (1) as antagonists of ion channels from the Kv family and/or of the biological functions or pathways associated therewith, i.e. in an in vitro, in vivo or therapeutic setting; (2) as blockers of ion channels from the Kv family, i.e. in an in vitro, in vivo or therapeutic setting; and/or (3) as pharmaceutically active agents, in particular in (the preparation of pharmaceutical compositions for) the prevention and/or treatment of conditions or diseases associated with ion channels from the Kv family.

More in particular, the compounds of the invention that interact with ion channels from the Kv4 subfamily can be used (1) as antagonists of ion channels from the Kv4 subfamily and/or of the biological functions or pathways associated therewith, i.e. in an in vitro, in vivo or therapeutic setting; (2) as blockers of ion channels from the Kv4 subfamily, i.e. in an in vitro, in vivo or therapeutic setting; and/or (3) as pharmaceutically active agents, in particular in (the preparation of pharmaceutical compositions for) the prevention and/or treatment of conditions or diseases associated with ion channels from the Kv4 sub family.

Even more in particular, the compounds of the invention that interact with the Kv4.3 ion channels from the Kv4 subfamily can in particular be used (1) as antagonists of the Kv4.3 ion channel and/or of the biological functions or pathways associated therewith, i.e. in an in vitro, in vivo or therapeutic setting; (2) as blockers of the Kv4.3 ion channel, i.e. in an in vitro, in vivo or therapeutic setting; and/or (3) as pharmaceutically active agents, in particular in (the preparation of pharmaceutical compositions for) the prevention and/or treatment of conditions or diseases associated with the Kv4.3 ion channel.

According to a further embodiment, the compounds of the invention that interact with ion channels from the Kv1 subfamily can be used (1) as antagonists of ion channels from the Kv1 subfamily and/or of the biological functions or pathways associated therewith, i.e. in an in vitro, in vivo or therapeutic setting; (2) as blockers of ion channels from the Kv1 subfamily, i.e. in an in vitro, in vivo or therapeutic setting; and/or (3) as pharmaceutically active agents, in particular in (the preparation of pharmaceutical compositions for) the prevention and/or treatment of conditions or diseases associated with ion channels from the Kv1 sub family.

More in particular, the compounds of the invention that interact with the Kv 1.5 ion channels from the Kv1 subfamily can in particular be used (1) as antagonists of the Kv1.5 ion channel and/or of the biological functions or pathways associated therewith, i.e. in an in vitro, in vivo or therapeutic setting; (2) as blockers of the Kv1.5 ion channel, i.e. in an in vitro, in vivo or therapeutic setting; and/or (3) as pharmaceutically active agents, in particular in (the preparation of pharmaceutical compositions for) the prevention and/or treatment of conditions or diseases associated with the Kv1.5 ion channel.

In a further aspect, the present invention provides a compound of Formula I, II, III or IV for use as a medicament. Furthermore, the present invention provides a compound of Formula I, II, III or IV for use as an ion channel blocker. In addition, the present invention provides a compound of Formula I, II, III or IV for use as a blocker of an ion-channel of the Kv4 family of ion channels. In particular, the present invention provides a compound of Formula I, II, III or IV for use as a blocker of an ion-channel of the Kv4.3 family of ion-channels. Further, the present invention provides a compound of Formula I, II, III or IV for use as a blocker of an ion channel of the Kv1 family of ion channels. In particular, the present invention provides a compound of Formula I, II, III or IV for use as a blocker of an ion channel of the Kv1.5 family of ion channels.

The present invention further provides for the use of a compound according to the invention for the preparation of a medicament in the prevention and/or treatment of conditions or diseases associated with ion channels of the Kv4 and/or Kv1 family.

Such diseases and disorders will be clear to the skilled person. For example, conditions and diseases associated with the Kv4.3 ion channel, in particular in humans, include cardiac disorders such as arrhythmia, hypertension-induced heart disorders such as hypertension-induced cardiac hypertrophy (e.g. ventricular hypertrophy), and disorders of the nervous system such as neurological disorders non-limiting examples of which include epilepsy, stroke, traumatic brain injury, anxiety, insomnia, Alzheimer's disease, spinal cord injury, encephalomyelitis, multiple sclerosis, demyelinating disease, and Parkinson's syndrome; and the compounds of the invention that interact with Kv4.3 ion channels can be used in the prevention and/or treatment of such conditions and diseases.

Similar conditions and diseases are associated with the Kv1.5 ion channel and can be used in prevention and/or treatment of such conditions and diseases. For instance, class III anti-arrhythmic drugs exert their effects by a blockade of cardiac potassium channels, resulting in a prolongation of repolarization and refractoriness. I(Kur), the ultra-rapid delayed rectifier current was identified in human atrial but not ventricular tissue. Consequently, it contributes to the repolarisation of the action potential in the atrium only. The Kv1.5 protein is supposed to be a critical cardiac voltage-gated potassium channel to form the I(Kur). Compounds inhibiting Kv1.5 would delay repolarisation of the action potential in the atrium and consequently prolong the atrial refractory period. Assuming high selectivity of a Kv1.5 inhibitor over hERG, such inhibitor would not interfere with ventricular repolarization, which has been associated with pro-arrhythmia, e.g. torsades de pointes. Therefore, Kv1.5 inhibitors are of special interest in the treatment of atrial tachyarrhythmias such as atrial fibrillation. Therefore, according to a further embodiment, the present invention also relates to the use of the compounds that interact with Kv1.5 ion channels for prevention and/or treatment of the conditions and diseases given above and related with Kv4.3 ion channel associated diseases. Preferred compounds for use in treating these conditions or diseases are compounds that show activity for both the Kv4.3 and the Kv1.5 ion channel. For example, the compounds are suitable for the treatment and/or prevention of various disorders: cardiac arrhythmias, including supraventricular arrhythmias, atrial arrhythmias, atrial fibrillation, atrial flutters, complications of cardiac ischemia. The compounds may also, for example, be employed for the termination of existing atrial fibrillation or flutters for the recovery of the sinus rhythm (cardio version). Moreover, the substances may reduce the susceptibility to the formation of new fibrillation events (maintenance of the sinus rhythm, prophylaxis).

The compounds according to the invention can also be used as heart rate control agents, angina pectoris including relief of Prinzmetal's symptoms, vasospastic symptoms and variant symptoms; gastrointestinal disorders including reflux, esophagitis, functional dispepsia, motility disorders (including constipation and diarrhea), and irritable bowel syndrome, disorders of vascular and visceral smooth muscle including asthma, chronic obstructive pulmonary disease, adult respiratory distress syndrome, peripheral vascular disease (including intermittent claudication), venous insufficiency, impotence, cerebral and coronary spasm and Raynaud's disease, inflammatory and immunological disease including inflammatory bowel disease, rheumatoid arthritis, graft rejection, asthma. chronic obstructive pulmonary disease, cystic fibrosis and atherosclerosis, cell poliferative disorders including restenosis and cancer (including leukemia), disorders of the auditory system, disorders of the visual system including macular degeneration and cataracts, diabetes including diabetic retinopathy, diabetic nephropathy and diabetic neuropathy, muscle disease including myotonia and wasting, peripheral neuropathy, cognitive disorders, migraine, memory loss including Alzheimer's and dementia, spinal cord injury, encephalomyelitis, multiple sclerosis, demyelinating disease, CNS mediated motor dysfunction including Parkinson's disease, and ataxia, epilepsy, and other ion channel mediated disorders.

As inhibitors of the K1 subfamily of voltage-gated K+ channels compounds according to the present invention are useful to treat a variety of disorders including resistance by transplantation of organs or tissue, graft-versus-host diseases brought about by medulla ossium transplantation, rheumatoid arthritis, systemic lupus erythematosus, hashimoto's thyroiditis, multiple sclerosis, myasthenia gravis, type I diabetes uveitis, juvenile-onset or recent-onset diabetes mellitus, posterior uveitis, allergic encephalomyelitis, glomerulonephritis, infectious diseases caused by pathogenic microorganisms, inflammatory and hyperproliferative skin diseases, psoriasis, atopical dermatitis, contact dermatitis, eczematous dermatitises, seborrhoeis dermatitis, Lichen planus, Pemphigus, bullous pemphigoid, Epidermolysis bullosa, urticaria, angioedemas, vasculitides, erythemas, cutaneous eosinophilias, Lupuserythematosus, acne, Alopecia greata, keratoconjunctivitis, vernal conjunctivitis, uveitis associated with Behcet's disease, keratitis, herpetic keratitis, conical cornea, dystrophia epithelialis corneae, corneal leukoma, ocular pemphigus, Mooren's ulcer, Scleritis, Graves' opthalmopathy, Vogt-Koyanagi-Harada syndrome, sarcoidosis, pollen allergies, reversible obstructive airway disease, bronchial asthma, allergic asthma, intrinsic asthma, extrinsic asthma, dust asthma, chronic or inveterate asthma, late asthma and airway hyper-responsiveness, bronchitis, gastric ulcers, vascular damage caused by ischemic diseases and thrombosis, ischemic bowel diseases, inflammatory bowel diseases, necrotizing enterocolitis, intestinal lesions associated with thermal burns and leukotriene B4-mediated diseases, Coeliaz diseases, proctitis, eosinophilic gastroenteritis, mastocytosis, Crohn's disease, ulcerative colitis, migraine, rhinitis, eczema, interstitial nephritis, Good-pasture's syndrome, hemolyticuremic syndrome, diabetic nephropathy, multiple myositis, Guillain-Barre syndrome, Meniere's disease, polyneuritis, multiple neuritis, mononeuritis, radiculopathy, hyperthroidism, Basedow's disease, pure red cell aplasia, aplastic anemia, hypoplastic anemia, idiopathic thrombocytopenic purpura, autoimmune hemolytic anemia, agranulocytosis, pernicious anemia, megaloblastic anemia, anerythroplasia, osteoporosis, sarcoidosis, fibroid lung, idopathic interstitial pneumonia, dermatomyositis, leukoderma vulgaris, ichthyosis vulgaris, photoallergic sensitivity, cutaneous T cell lymphoma, arteriosclerosis, atherosclerosis, aortitis syndrome, polyarteritis nodosa, myocardosis, scleroderma, Wegener's granuloma, Sjogren's syndrome, adiposis, eosinophilic fascitis, lesions of gingiva, periodontium, alveolar bone, substantia osses dentis, glomerulonephritis, male pattern alopecia or alopecia senilis by preventing epilation or providing hair germination and/or promoting hair generation and hair growth, muscular dystrophy, Pyoderma and Sezary's syndrome, Addison's disease, ischemia-reperfusion injury of organs which occurs upon preservation, transplantation or ischemic disease, endotoxin-shock, pseudomembranous colitis, colitis caused by drug or radiation, ischemic acute renal insufficiency, chronic renal insufficiency, toxinosis caused by lung-oxygen or drugs, lung cancer, pulmonary emphysema, cataracta, siderosis, retinitis, pigentosa, senile macular degeneration, vitreal scarring, corneal alkali burn, dermatitis erythema multiforme, linear IgA ballous dermatitis and cement dermatitis, gingivitis, periodontitis, sepsis, pancreatitis, diseases caused by environmental pollution, aging, carcinogenis, metastatis of carcinoma and hypobaropathy, disease caused by histamine or leukotriene-C4 release, Behcet's disease, autoimmune hepatitis, primary biliary cirrhosis sclerosing cholangitis, partial liver resection, acute liver necrosis, necrosis caused by toxin, viral hepatitis, shock, or anoxia, B-virus hepatitis, nonA/non-B hepatitis, cirrhosis, alcoholic cirrhosis, hepatic failure, fulminant hepatic failure, late-onset hepatic failure, “acute-on-chronic” liver failure, augmentation of chemotherapeutic effect, cytomegalovirus infection, HCMV infection, AIDS, cancer, senile dementia, trauma, and chronic bacterial infection.

The compounds of the present invention are antiarrhythmic agents which are useful in the prevention and treatment (including partial alleviation or cure) of arrhythmias. As inhibitors of Kv1.5, compounds within the scope of the present invention are particularly useful in the selective prevention and treatment of supraventricular arrhythmias such as atrial fibrillation, and atrial flutter.

Whether a compound of the invention interacts with an ion channel, such as with an ion channel of the Kv family, for example an ion channel of the Kv4 or Kv1 subfamily, such as the Kv4.3 or the Kv1.5 ion channel, respectively, can be determined using a suitable technique or assay, such as the assays and techniques referred to herein or other suitable assays or techniques known in the art.

For pharmaceutical use, the compounds of the invention may be used as a free acid or base, and/or in the form of a pharmaceutically acceptable acid-addition and/or base-addition salt (e.g. obtained with non-toxic organic or inorganic acid or base), in the form of a hydrate, solvate and/or complex, and/or in the form or a pro-drug or pre-drug, such as an ester. As used herein and unless otherwise stated, the term “solvate” includes any combination which may be formed by a compound of this invention with a suitable inorganic solvent (e.g. hydrates) or organic solvent, such as but not limited to alcohols, ketones, esters and the like. Such salts, hydrates, solvates, etc. and the preparation thereof will be clear to the skilled person; reference is for instance made to the salts, hydrates, solvates, etc. described in U.S. Pat. No. 6,372,778, U.S. Pat. No. 6,369,086, U.S. Pat. No. 6,369,087 and U.S. Pat. No. 6,372,733.

The pharmaceutically acceptable salts of the compounds according to the invention, i.e. in the form of water-, oil-soluble, or dispersible products, include the conventional non-toxic salts or the quaternary ammonium salts which are formed, e.g., from inorganic or organic acids or bases. Examples of such acid addition salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalene-sulfonate, nicotinate, oxalate, pamoate, pectinate, persulfate, 3-phenylpropionate, picrate, pivalate, propionate, succinate, tartrate, thiocyanate, tosylate, and undecanoate. Base salts include ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such a sarginine, lysine, and so forth. Also, the basic nitrogen-containing groups may be quaternized with such agents as lower alkyl halides, such as methyl, ethyl, propyl, and butyl chloride, bromides and iodides; dialkyl sulfates like dimethyl, diethyl, dibutyl; and diamyl sulfates, long chain halides such as decyl, lauryl, myristyl and stearyl chlorides, bromides and iodides, aralkyl halides like benzyl and phenethyl-bromides and others. Other pharmaceutically acceptable salts include the sulfate salt ethanolate and sulfate salts.

In another embodiment, the present invention relates to a pharmaceutical composition comprising a pharmaceutically acceptable excipient and a therapeutic amount of a compound according to the invention.

The term “therapeutically effective amount” as used herein means that amount of active compound or component or pharmaceutical agent that elicits the biological or medicinal response in a tissue, system, animal or human that is being sought by a researcher, veterinarian, medical doctor or other clinician, which includes alleviation of the symptoms of the disease being treated.

The pharmaceutical composition can be prepared in a manner known per se to one of skill in the art. For this purpose, at least one compound having Formula I, II, III or IV, one or more solid or liquid pharmaceutical excipients and, if desired, in combination with other pharmaceutical active compounds, are brought into a suitable administration form or dosage form which can then be used as a pharmaceutical in human medicine or veterinary medicine.

Generally, for pharmaceutical use, the compounds of the inventions may be formulated as a pharmaceutical preparation comprising at least one compound of the invention and at least one pharmaceutically acceptable carrier, diluent or excipient and/or adjuvant, and optionally one or more further pharmaceutically active compounds. By means of non-limiting examples, such a formulation may be in a form suitable for oral administration, for parenteral administration (such as by intravenous, intramuscular or subcutaneous injection or intravenous infusion), for topical administration, for administration by inhalation, by a skin patch, by an implant, by a suppository, etc. Such suitable administration forms—which may be solid, semi-solid or liquid, depending on the manner of administration—as well as methods and carriers, diluents and excipients for use in the preparation thereof, will be clear to the skilled person; reference is again made to for instance U.S. Pat. No. 6,372,778, U.S. Pat. No. 6,369,086, U.S. Pat. No. 6,369,087 and U.S. Pat. No. 6,372,733, as well as to the standard handbooks, such as the latest edition of Remington's Pharmaceutical Sciences.

Some preferred, but non-limiting examples of such preparations include tablets, pills, powders, lozenges, sachets, cachets, elixirs, suspensions, emulsions, solutions, syrups, aerosols, ointments, creams, lotions, soft and hard gelatin capsules, suppositories, sterile injectable solutions and sterile packaged powders (which are usually reconstituted prior to use) for administration as a bolus and/or for continuous administration, which may be formulated with carriers, excipients, and diluents that are suitable per se for such formulations, such as lactose, dextrose, sucrose, sorbitol, mannitol, starches, gum acacia, calcium phosphate, alginates, tragacanth, gelatin, calcium silicate, microcrystalline cellulose, polyvinylpyrrolidone, polyethylene glycol, cellulose, (sterile) water, methylcellulose, methyl- and propylhydroxybenzoates, talc, magnesium stearate, edible oils, vegetable oils and mineral oils or suitable mixtures thereof. The formulations can optionally contain other pharmaceutically active substances (which may or may not lead to a synergistic effect with the compounds of the invention) and other substances that are commonly used in pharmaceutical formulations, such as lubricating agents, wetting agents, emulsifying and suspending agents, dispersing agents, desintegrants, bulking agents, fillers, preserving agents, sweetening agents, flavoring agents, flow regulators, release agents, and the like. The compositions may also be formulated so as to provide rapid, sustained or delayed release of the active compound(s) contained therein, for example using liposomes or hydrophilic polymeric matrices based on natural gels or synthetic polymers.

In order to enhance the solubility and/or the stability of the compounds of a pharmaceutical composition according to the invention, it can be advantageous to employ α-, β- or γ-cyclodextrins or their derivatives. In addition, co-solvents such as alcohols may improve the solubility and/or the stability of the compounds. In the preparation of aqueous compositions, addition of salts of the compounds of the invention can be more suitable due to their increased water solubility.

Appropriate cyclodextrins are α-, β- or γ-cyclodextrins (CDs) or ethers and mixed ethers thereof wherein one or more of the hydroxy groups of the anhydroglucose units of the cyclodextrin are substituted with alkyl, particularly methyl, ethyl or isopropyl, e.g. randomly methylated 81-CD; hydroxyalkyl, particularly hydroxyethyl, hydroxypropyl or hydroxybutyl; carboxyalkyl, particularly carboxymethyl or carboxyethyl; alkylcarbonyl, particularly acetyl; alkoxycarbonylalkyl or carboxyalkoxyalkyl, particularly carboxymethoxypropyl or carboxyethoxypropyl; alkylcarbonyloxyalkyl, particularly 2-acetyloxypropyl. Especially noteworthy as complexants and/or solubilizers are β-CD, randomly methylated β-CD, 2,6-dimethyl-β-CD, 2-hydroxyethyl-β-CD, 2-hydroxyethyl-γ-CD, 2-hydroxypropyl-γ-CD and (2-carboxymethoxy)propyl-β-CD, and in particular 2-hydroxypropyl-γ-CD (2-HP-β-CD). The term mixed ether denotes cyclodextrin derivatives wherein at least two cyclodextrin hydroxy groups are etherified with different groups such as, for example, hydroxypropyl and hydroxyethyl. An interesting way of formulating the compounds in combination with a cyclodextrin or a derivative thereof has been described in EP-A-721,331. Although the formulations described therein are with antifungal active ingredients, they are equally interesting for formulating the compounds. Said formulations may also be rendered more palatable by adding pharmaceutically acceptable sweeteners and/or flavors. In particular, the present invention encompasses a pharmaceutical composition comprising an effective amount of a compound according to the invention with a pharmaceutically acceptable cyclodextrin. The present invention also encompasses cyclodextrin complexes consisting of a compound according to the invention and a cyclodextrin.

More in particular, the compositions may be formulated in a pharmaceutical formulation comprising a therapeutically effective amount of particles consisting of a solid dispersion of the compounds of the invention and one or more pharmaceutically acceptable water-soluble polymers.

The term “a solid dispersion” defines a system in a solid state (as opposed to a liquid or gaseous state) comprising at least two components, wherein one component is dispersed more or less evenly throughout the other component or components. When said dispersion of the components is such that the system is chemically and physically uniform or homogenous throughout or consists of one phase as defined in thermodynamics, such a solid dispersion is referred to as “a solid solution”. Solid solutions are preferred physical systems because the components therein are usually readily bioavailable to the organisms to which they are administered. The term “a solid dispersion” also comprises dispersions that are less homogenous throughout than solid solutions. Such dispersions are not chemically and physically uniform throughout or comprise more than one phase.

The water-soluble polymer is conveniently a polymer that has an apparent viscosity of 1 to 100 mPa·s when dissolved in a 2% aqueous solution at 20° C. solution. Preferred water-soluble polymers are hydroxypropyl methylcelluloses or HPMC. HPMC having a methoxy degree of substitution from about 0.8 to about 2.5 and a hydroxypropyl molar substitution from about 0.05 to about 3.0 are generally water soluble. Methoxy degree of substitution refers to the average number of methyl ether groups present per anhydroglucose unit of the cellulose molecule. Hydroxy-propyl molar substitution refers to the average number of moles of propylene oxide which have reacted with each anhydroglucose unit of the cellulose molecule.

It may further be convenient to formulate the compounds in the form of nanoparticles which have a surface modifier adsorbed on the surface thereof in an amount sufficient to maintain an effective average particle size of less than 1000 nm. Suitable surface modifiers can preferably be selected from known organic and inorganic pharmaceutical excipients. Such excipients include various polymers, low molecular weight oligomers, natural products and surfactants. Preferred surface modifiers include nonionic and anionic surfactants.

Yet another interesting way of formulating the compounds according to the invention involves a pharmaceutical composition whereby the compounds are incorporated in hydrophilic polymers and applying this mixture as a coat film over many small beads, thus yielding a composition with good bio-availability which can conveniently be manufactured and which is suitable for preparing pharmaceutical dosage forms for oral administration. Said beads comprise (a) a central, rounded or spherical core, (b) a coating film of a hydrophilic polymer and an antiretroviral agent and (c) a seal-coating polymer layer. Materials suitable for use as cores in the beads are manifold, provided that said materials are pharmaceutically acceptable and have appropriate dimensions and firmness. Examples of such materials are polymers, inorganic substances, organic substances, and saccharides and derivatives thereof.

The above preparations may be prepared in a manner known per se, which usually involves mixing the active substance(s) to be used with the one or more pharmaceutically acceptable carriers, which necessary under aseptic conditions. Reference is again made to U.S. Pat. No. 6,372,778, U.S. Pat. No. 6,369,086, U.S. Pat. No. 6,369,087 and U.S. Pat. No. 6,372,733 and the further prior art mentioned above, as well as to the standard handbooks, such as the latest edition of Remington's Pharmaceutical Sciences.

The pharmaceutical preparations of the invention are preferably in a unit dosage form, and may be suitably packaged, for example in a box, blister, vial, bottle, sachet, ampoule or in any other suitable single-dose or multi-dose holder or container (which may be properly labeled); optionally with one or more leaflets containing product information and/or instructions for use. Generally, such unit dosages will contain between 1 and 1000 mg, and usually between 5 and 500 mg, of the at least one compound of the invention, e.g. about 10, 25, 50, 100, 200, 300 or 400 mg per unit dosage.

The compounds can be administered by a variety of routes including the oral, rectal, transdermal, subcutaneous, intravenous, intrapericardial, intramuscular or intranasal routes, depending mainly on the specific preparation used and the condition to be treated or prevented, and with oral and intravenous administration usually being preferred.

The compound of the invention will generally be administered in an effective amount, which, upon suitable administration, is sufficient to achieve the desired therapeutic or prophylactic effect in the individual to which it is administered. Usually, depending on the condition to be prevented or treated and the route of administration, such an effective amount will usually be between 0.01 to 1000 mg, more often between 0.1 and 500 mg, such as between 0.1 and 250 mg, for example about 0.1, 1, 5, 10, 20, 50, 100, 150, 200 or 250 mg, per kilogram body weight day of the patient per day, which may be administered as a single daily dose, divided over one or more daily doses, or essentially continuously, e.g. using a drip infusion. The amount(s) to be administered, the route of administration and the further treatment regimen may be determined by the treating clinician, depending on factors such as the age, gender and general condition of the patient and the nature and severity of the disease/symptoms to be treated. Reference is again made to U.S. Pat. No. 6,372,778, U.S. Pat. No. 6,369,086, U.S. Pat. No. 6,369,087 and U.S. Pat. No. 6,372,733 and the further prior art mentioned above, as well as to the standard handbooks, such as the latest edition of Remington's Pharmaceutical Sciences. It will be understood, however, that specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition.

Thus, in a further aspect, the invention relates to a composition and in particular a composition for pharmaceutical use, which contains at least one compound of the invention and at least one suitable carrier (i.e. a carrier suitable for pharmaceutical use). The invention also relates to the use of a compound of the invention in the preparation of such a composition.

In accordance with the method of the present invention, said pharmaceutical composition can be administered separately at different times during the course of therapy or concurrently in divided or single combination forms. The present invention is therefore to be understood as embracing all such regimes of simultaneous or alternating treatment and the term “administering” is to be interpreted accordingly.

For an oral administration form, the compositions of the present invention can be mixed with suitable additives, such as excipients, stabilizers or inert diluents, and brought by means of the customary methods into the suitable administration forms, such as tablets, coated tablets, hard capsules, aqueous, alcoholic, or oily solutions. Examples of suitable inert carriers are gum arabic, magnesia, magnesium carbonate, potassium phosphate, lactose, glucose, or starch, in particular, corn starch. In this case, the preparation can be carried out both as dry and as moist granules. Suitable oily excipients or solvents are vegetable or animal oils, such as sunflower oil or cod liver oil. Suitable solvents for aqueous or alcoholic solutions are water, ethanol, sugar solutions, or mixtures thereof. Polyethylene glycols and polypropylene glycols are also useful as further auxiliaries for other administration forms. As immediate release tablets, these compositions may contain microcrystalline cellulose, dicalcium phosphate, starch, magnesium stearate and lactose and/or other excipients, binders, extenders, disintegrants, diluents and lubricants known in the art.

When administered by nasal aerosol or inhalation, these compositions may be prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. Suitable pharmaceutical formulations for administration in the form of aerosols or sprays are, for example, solutions, suspensions or emulsions of the compounds of the invention or their physiologically tolerable salts in a pharmaceutically acceptable solvent, such as ethanol or water, or a mixture of such solvents. If required, the formulation can also additionally contain other pharmaceutical auxiliaries such as surfactants, emulsifiers and stabilizers as well as a propellant.

For subcutaneous or intravenous administration, the compound according to the invention, if desired with the substances customary therefore such as solubilizers, emulsifiers or further auxiliaries are brought into solution, suspension, or emulsion. The compounds of the invention can also be lyophilized and the lyophilizates obtained used, for example, for the production of injection or infusion preparations. Suitable solvents are, for example, water, physiological saline solution or alcohols, e.g. ethanol, propanol, glycerol, in addition also sugar solutions such as glucose or mannitol solutions, or alternatively mixtures of the various solvents mentioned. The injectable solutions or suspensions may be formulated according to known art, using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid.

When rectally administered in the form of suppositories, these formulations may be prepared by mixing the compounds according to the invention with a suitable non-irritating excipient, such as cocoa butter, synthetic glyceride esters or polyethylene glycols, which are solid at ordinary temperatures, but liquefy and/or dissolve in the rectal cavity to release the drug.

The compounds according to the invention were found to act as antagonist of ion channels from the Kv family more in particular from the Kv4 subfamily and/or of the biological functions or pathways associated therewith. The compounds according to the invention were also found to act as antagonist of ion channels from the Kv1 subfamily and/or of the biological functions or pathways associated therewith.

The compounds of the invention can therefore be used (1) as antagonists of ion channels and/or of the biological functions or pathways associated therewith, i.e. in an vitro, in vivo or therapeutic setting; (2) as blockers of ion channels, i.e. in an vitro, in vivo or therapeutic setting; and/or (3) as pharmaceutically active agents, in particular in (the preparation of pharmaceutical compositions for) the prevention and/or treatment of conditions or diseases associated with said ion channels. In addition the compounds according to the invention showed very low activity or no activity with respect to the hERG channel, and are thereby selective.

As indicated above, due to the blocking activity on the above mentioned ion channels the compounds according to the present invention are particularly useful in the prevention and/or treatment of conditions or diseases associated with ion channels from the Kv family. Such diseases and disorders will be clear to the skilled person. For example, conditions and diseases associated with the Kv4.3 ion channel, in particular in humans, include cardiac disorders such as arrhythmia, hypertension-induced heart disorders such as hypertension-induced cardiac hypertrophy (e.g. ventricular hypertrophy), and disorders of the nervous system such as epilepsy, stroke, traumatic brain injury, anxiety, insomnia, spinal cord injury, encephalomyelitis, multiple sclerosis, demyelinating disease, Alzheimer's disease and Parkinson's syndrome. The compounds according to the present invention interact with Kv 4.3 ion channels and can be used in the prevention and/or treatment of such conditions and diseases. In addition, conditions and diseases associated with the Kv1.5 ion channel, in particular in humans, include the same diseases and disorders as mentioned above as for the Kv4.3 ion channel. The compounds according to the invention that interact with Kv1.5 ion channel are particularly useful in the prevention and/or treatment of atrial tachyarrhythmias such as atrial fibrillation.

Therefore, in another embodiment, the present invention also relates to the use of the compounds according to the invention or to a pharmaceutical composition comprising said compounds in the treatment of cardiac disorders such as arrhythmia, hypertension-induced heart disorders such as hypertension-induced cardiac hypertrophy (e.g. ventricular hypertrophy), and disorders of the nervous system such as epilepsy, stroke, traumatic brain injury, anxiety, insomnia, spinal cord injury, encephalomyelitis, multiple sclerosis, demyelinating disease, Alzheimer's disease and Parkinson's syndrome. In a further embodiment, the present invention also relates to the use of the compounds according to the invention or to a pharmaceutical composition comprising said compounds in the treatment of cardiac disorders such as arrhythmia. In another further embodiment, the present invention also relates to the use of the compounds according to the invention or to a pharmaceutical composition comprising said compounds in the treatment of disorders of the nervous system.

A method of treating cardiac disorders comprises administering to an individual in need of such treatment a pharmaceutical composition comprising the compounds according to the invention. A method of treating disorders of the nervous system comprises administering to an individual in need of such treatment a pharmaceutical composition comprising the compounds according to the invention.

It is also envisaged that the above compounds and compositions may be of value in the veterinary field, which for the purposes herein not only includes the prevention and/or treatment of diseases in animals, but also—for economically important animals such as cattle, pigs, sheep, chicken, fish, etc.—enhancing the growth and/or weight of the animal and/or the amount and/or the quality of the meat or other products obtained from the animal. Thus, in a further aspect, the invention relates to a composition for veterinary use that contains at least one compound of the invention (i.e. a compound that has been identified, discovered and/or developed using a nematode or method as described herein) and at least one suitable carrier (i.e. a carrier suitable for veterinary use). The invention also relates to the use of a compound of the invention in the preparation of such a composition. It is also envisaged that the above compounds and compositions may be of value as insecticides.

The invention will now be illustrated by means of the following synthetic and biological examples, which do not limit the scope of the invention in any way.

### EXAMPLES

### Example 1

### Preparation of the Compounds According to the Present Invention

The practice of the present invention will employ, unless otherwise indicated, conventional techniques of synthetic organic chemistry, biological testing, and the like, which are within the skill of the art. Such techniques are explained fully in the literature. Unless indicated otherwise, the purity of the compounds was confirmed by liquid chromatography/mass spectrometry (LC/MS), according to method A:

Method A:

HPLC: Waters Alliance 2690 with photodiode array detector Waters 996. Mass spectrometer: Micromass Platform ZMD LC. Ionization: electrospray (polarity: negative and positive).

Method:

Phase: Tosohaas TSK-gel super ODS (100 Å, 2 μm), column: 4.6×50 mm; Solvent A: Water and formic acid (26.5 mM); Solvent B: Acetonitrile and formic acid (17 mM); Flow: 2.75 ml/min; Gradient 5 min: From 100% A & 0% B to 20% A & 80% B in 3 min. Isocratic 80% B for 1 min. From 80% B & 20% A to 0% B and 100% A in 0.5 min. Isocratic 100% A for 0.5 min

NMR spectra were determined on a Varian Mercury 300 MHz NMR using the indicated solvent as an internal reference.

Melting points were determined on a Büchi B-540 and are non-corrected. All reagents used either were obtained commercially or were prepared in a manner known per se.

Methods of Preparation

Compounds of Formula I, II, III or IV may be prepared according to the following protocols and schemes and the knowledge of one skilled in the art.

Protocol A:

The acid derivative (0.5 mmol) was dissolved in a mixture of DMF (0.5 ml) and DIEA (1.5 mmol). A solution of TBTU (0.5 mmol) and HOBt (0.1 mmol) in DMF (0.5 ml) was added and the mixture was stirred at room temperature for 30 minutes. The amine (0.5 mmol) was added and the reaction mixture was stirred at room temperature for a period of 3 to 24 hours. DMF was removed under reduced pressure. The residue (0.5 mmol) was diluted with EtOAc (5 ml) or DCM (5 ml) and washed with 0.5N HCl (2×5 ml), 0.5N NaOH (2×5 ml) and water (2×5 ml) or with 1N NaHCO₃ (2×5 ml) and water (2×5 ml). The organic layer was dried over MgSO₄ and the solvent was evaporated under vacuum. The residue was purified by flash chromatography, semi-prep HPLC or recrystallization.

Synthesis of 5-chlorobenzofurane-2-carboxylic acid (R)-(4-nitrophen-1-yl)ethylamide

(compound 1) is given as example.

In a round bottom flask 5-chlorobenzofurane-2-carboxylic acid (98 mg; 0.5 mmoles) was dissolved in a solution of DIEA (261 μl; 1.5 mmoles) in DMF (0.5 ml). A solution of TBTU (160 mg; 0.5 mmoles) and HOBT (14 mg; 0.1 mmole) in DMF (0.5 ml) was added and the reaction was stirred for 30 min at room temperature. (R)-(4-nitrophen-1-yl)ethylamine (76 mg; 0.5 mmoles) was then added (if the amine was stocked as hydrochloride, 1 more equivalent of DIEA was needed). After 2 hours of stirring at room temperature, a solution of TBTU (112 mg; 0.35 mmoles) and HOBT (14 mg; 0.1 mmole) in DMF (0.35 ml) was added. Stirring was continued at room temperature for 4 hours. DMF was then removed under vacuum. The residue was dissolved in ethylacetate (5 ml). The organic layer was washed with HCl 0.5N (2×5 ml), NaOH 0.5N (2×5 ml), and water (2×5 ml). The organic layer was then dried (magnesium sulfate) and evaporated under vacuum. The residue was purified by preparative HPLC.

Protocol B:

SOCl₂ (5 ml) and DMF (2 drops) were added to the carboxylic acid (3.08 mmol) and the mixture was stirred at 45° C. for 30 minutes. The excess of SOCl₂ was removed under reduced pressure. Traces of SOCl₂ were removed by distillation from DCM (2×5 ml). The acyl chloride was dissolved in DCM (5 ml) and added at 0° C. under nitrogen atmosphere to a stirred mixture of the amine (3.08 mmol) and Et₃N (18.5 mmol) or DIEA (18.5 mmol) in DCM (5 ml). The mixture was stirred at 0° C. for 30 min and then allowed to warm up to room temperature. The reaction mixture was stirred at room temperature for a period of 30 minutes to 24 hours. The mixture was poured into water (100 ml) and extracted with DCM (3×100 ml). The combined organic phases were dried over MgSO₄ and the solvent was removed under reduced pressure. The residue was purified by flash chromatography, semi-preparative HPLC or recrystallization.

Protocol C:

The ester (1.7 mmol) was dissolved in ethanol (5 ml) and 2N NaOH (10 ml) was added. The reaction mixture was stirred at 45° C. for 30 minutes. The reaction mixture was cooled to room temperature and ethanol was removed under reduced pressure. The residue was diluted with water (10 ml), cooled to 0° C. and acidified to pH=1 using 6N HCl. The precipitate was filtered, washed with water (3×10 ml) and dried under reduced pressure.

Protocol D:

5-Chloroindole-2-carboxylic acid (2.5 mmol) was dissolved in CH₃CN (20 ml). DBU (6.5 mmol) and MeI (5.6 mmol) were added. The mixture was stirred at 65° C. for 8 hours. The solvent was removed under reduced pressure.

The residue (2.5 mmol) was dissolved in dry 1,4-dioxane (20 ml) and the solution was cooled to 0° C. NaH (8.1 mmol) was added and the mixture was stirred at 0° C. for 15 minutes. MeI (10 mmol) was added and the mixture was stirred at 0° C. for 1 hour and at room temperature for 2 days. The reaction mixture was poured into water (150 ml). The water layer was extracted with Et₂O (3×100 ml). The combined organic layers were washed with 1N HCl (3×200 ml), dried over MgSO₄ and the solvent was removed under reduced pressure. The residue was purified by flash chromatography.

Protocol E:

To a solution of benzofuran-2-carboxylic acid (39.1 mmol) in dry 1,4-dioxane (250 ml) was added Et₃N (58.7 mmol) and diphenylphosphoryl azide (50.8 mmol). The reaction mixture was stirred overnight at room temperature and under nitrogen atmosphere. EtOH (50 ml) was added and the reaction mixture was heated overnight at 100° C. The solvent was removed under reduced pressure and azeotropic distillation with toluene. The residue was crystallized from a toluene/EtOAc mixture.

Protocol F:

NaH (8.1 mmol) was added to a solution of (4-{[(5-chloro-benzofuran-2-carbonyl)-amino]-methyl}-phenyl)-carbamic acid ethyl ester (2.4 mmol) in dry 1,4-dioxane (20 ml). The reaction mixture was stirred at room temperature for 2 hours. A solution of (2,4-dimethoxy-phenyl)acetyl chloride (3.66 mmol) in dry 1,4-dioxane (10 ml) was added slowly. The reaction mixture was stirred overnight at room temperature and under nitrogen atmosphere. The reaction was quenched with a diluted NaOH solution (100 ml) and extracted with Et₂O (3×100 ml). The combined organic layers were washed with water (3×100 ml) and dried over MgSO₄. The solvent was removed under reduced pressure and the residue was purified by flash chromatography.

Protocol G:

20 ml of a 1.0 M methanolic solution of Mg(OMe)₂ was added to (4-{[(5-chloro-benzofuran-2-carbonyl)-amino]-methyl}-phenyl)-[2-(2,4-dimethoxy-phenyl)-acetyl]-carbamic acid ethyl ester (1.12 mmol) and the mixture was stirred overnight at room temperature. The reaction mixture was poured into a 20% aqueous solution of CH₃COOH (50 ml) and extracted with chloroform (3×100 ml). The combined organic layers were washed with aqueous NaOH solution (3×100 ml) and water (3×100 ml). The organic layer was dried over MgSO₄ and the solvent was removed under reduced pressure. The residue was purified by flash chromatography.

Protocol H:

A solution of benzofuran (29.6 mmol) in dry THF (20 ml) was cooled to −80° C. under nitrogen atmosphere. A 2.5 M solution of n-BuLi in hexane (32.5 mmol) was added and the mixture was stirred at −80° C. for 1 hour. A solution of (2,4-dimethoxyphenyl)acetic acid (12.7 mmol) in dry THF (20 ml) was added slowly and the mixture was slowly allowed to warm up to room temperature. After 4.5 h the reaction mixture was quenched with water (100 ml) and extracted with EtOAc (5×100 ml). The combined organic layers were washed with brine (3×300 ml), dried over MgSO₄ and the solvent was removed under reduced pressure. The residue was purified by flash chromatography.

Protocol I:

Boron tribromide (5.0 mmol) was added to a solution of 5-chloro-benzofuran-2-carboxylic acid 4-methoxy-benzylamide (0.83 mmol) in DCM (5 ml). The solution was stirred at −50° C. for 1 h20 and at room temperature for 2 hours. The reaction mixture was quenched with water (5 ml) and extracted with EtOAc (3×10 ml). The combined organic layers were dried over MgSO₄ and the solvent was removed under reduced pressure. The residue was purified by recrystallization.

Protocol J:

DIEA (0.44 mmol) and acetyl chloride (0.44 mmol) were added to a solution of 5-chloro-benzofuran-2-carboxylic acid 4-hydroxy-benzylamide (0.44 mmol) in DCM (3 ml). The solution was stirred at room temperature for 40 minutes. The reaction mixture was diluted with DCM (20 ml) and washed with a 1M solution of Na₂CO₃. The organic layer was dried over MgSO₄ and the solvent was removed under reduced pressure. The residue was purified by recrystallization.

Protocol K:

The ester (0.77 mmol) was dissolved in ethanol (3 ml) and 1N LiOH (0.77 mmol) was added. The mixture was stirred at 50° C. for 1 hour. The pH was adjusted to 2 with 1N HCl or poured into a 20% KHSO₄ aqueous solution. The precipitate was filtered, washed with water (2×20 ml) and dried under reduced pressure.

Protocol L:

5-Chloro-benzofuran-2-carboxylic acid (0.5 mmol) and benzylchloride (0.5 mmol) were dissolved in DMF (5 ml). K₂CO₃ (0.6 mmol) was added and the mixture was stirred at 65° C. for 17 hours. After cooling to room temperature, the solvent was removed under reduced pressure. The residue was washed with MeOH (2×20 ml) and the solvent of the filtrate was removed under reduced pressure. The residue was purified by flash chromatography.

Protocol M:

(4-Dimethylsulfamoyl-benzyl)-carbamic acid tert-butyl ester (0.65 mmol) was dissolved in a mixture of CH₃CN (2 ml) and 2N HCl (2 ml). The mixture was stirred overnight at 50° C. After cooling to room temperature, the solvent was removed under reduced pressure.

Protocol N:

3-Amino-5-chlorobenzofuran-2-carboxylic acid methyl ester (0.89 mmol) was dissolved in DCM (2 ml). DIEA (2.2 mmol) and acetic anhydride (1.8 mmol) were added. The mixture was stirred at room temperature for 48 hours. The solvent was removed under reduced pressure.

The present invention further encompasses compounds number 1 to 120 as illustrated in Table 12 as well as stereoisomers, tautomers, racemics, prodrugs, metabolites thereof, or a pharmaceutically acceptable salt and/or solvate thereof.

The present invention also encompasses the synthesis intermediates 11 to 19.

Compounds 1, 2, 3, 4, 5, 6, 7, 8, 24, 25, 26, 27, 28, 34, 36, 38, 39, 41, 44, 45, 49, 57, 58, 59, 60, 61, 62, 63, 64, 65, 66, 68, 69, 70, 71, 72, 73, 74, 75, 76, 77, 79, 80, 81, 82, 83, 84, 85, 87, 88, 89, 90, 91, 92, 93, 94, 95, 98, 99, 101, 102, 103, 104, 105, 108, 109, 113, 114, 118 and 119 were made from 5-chloro-benzofuran-2-carboxylic acid. Compounds 9, 10 and 11 were made from 7-methoxy-benzofuran-2-carboxylic acid.

Compound 13 was made from 4-acetyl-7-methoxy-benzofuran-2-carboxylic acid. Compounds 16 and 106 were made from 5-chloro-3-methyl-benzofuran-2-carboxylic acid. Compound 18 was made from 3-pyrrol-1-yl-benzofuran-2-carboxylic acid.

Compound 19 was made from 5-chloro-1H-indole-2-carboxylic acid according to scheme 1.

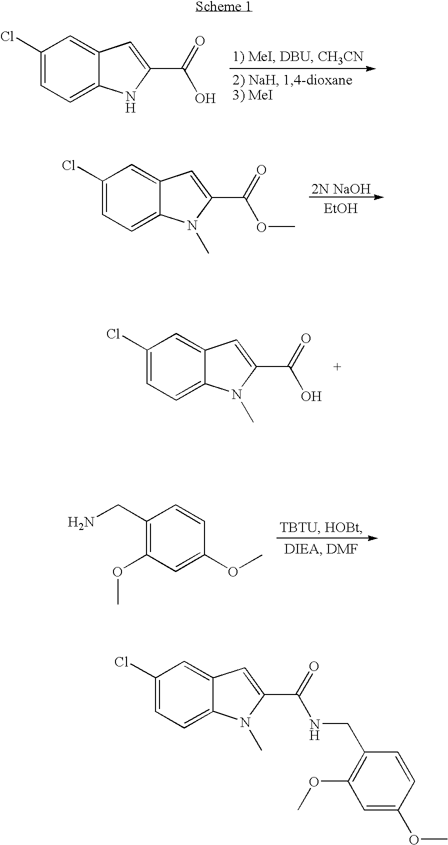

Compounds 21, 107, 110 and 117 were made from 5-chloro-3-methyl-benzo[b]thiophene-2-carboxylic acid.

Compounds 22 was made from benzofuran-2-carboxylic acid according to scheme 2.

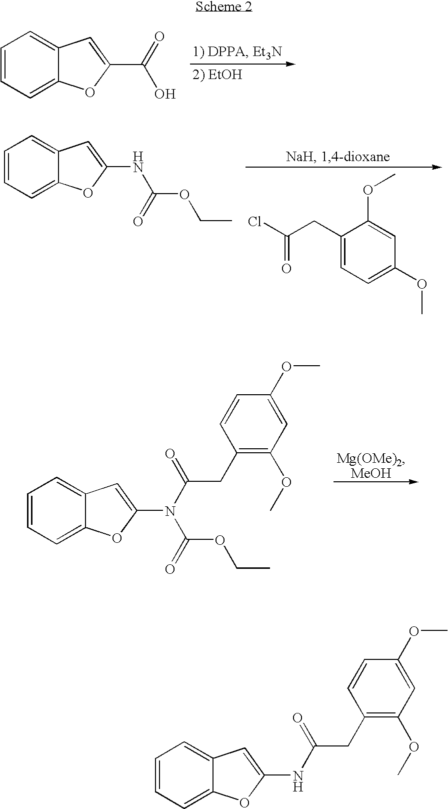

Compounds 23 and 50 were made from benzofuran-2-carboxylic acid. Compound 32 was made from benzo[b]thiophene-2-carboxylic acid. Compound 33 was made from the intermediates 14 and 15. Compound 42 was made from quinoline-3-carboxylic acid. Compound 43 was made from 3-methyl-benzofuran-2-carboxylic acid. Compound 67 was made from 5-chloro-benzofuran-2-carboxylic acid and intermediate 16. Compound 96 was made from 1-benzyl-1H-indole-3-carboxylic acid. Compound III was made from 5-chloro-1H-indole-2-carboxylic acid. Compound 112 was made from quinoline-2-carboxylic acid.

Compound 115 was made from 3-amino-5-chloro-benzofuran-2-carboxylic acid according to scheme 3.

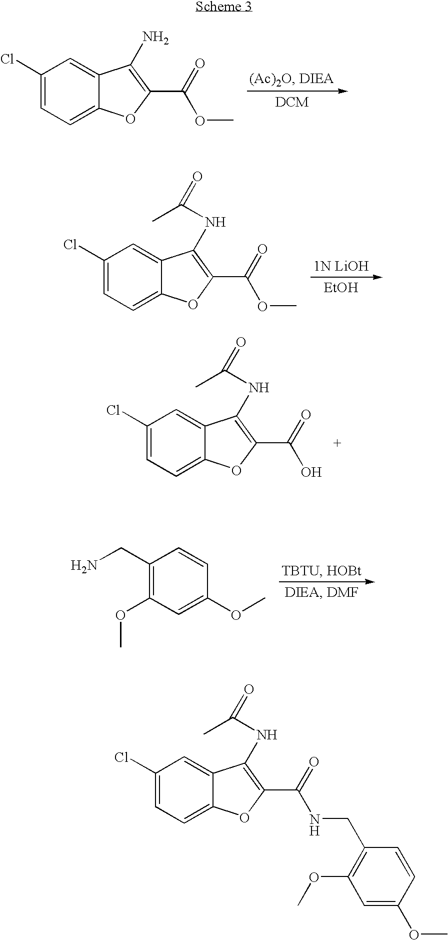

Compound 116 was made from 3-amino-5-chloro-benzofuran-2-carboxylic acid. Compound 120 was made from 5-chloro-benzooxazole-2-carboxylic acid.

The structural formulas of the intermediates are listed in Table 1. The following abbreviations are used hereunder. P: protocol, Rt: retention time, PU: purity. ES+: molecular ion obtained by electrospray in positive ion mode.

TABLE 1

| Name | Compound | Structure | P | Rt | PU | ES³⁰ |
|---|---|---|---|---|---|---|
| 5-chloro-1-methyl-1H- indole-2-carboxylic acid methyl ester | I1 | 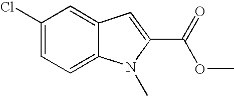 | D | 2.65 | 100 | 224 |
| benzofuran-2-yl- carbamic acid ethyl ester | I2 | 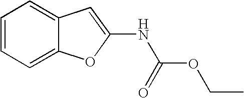 | E | ND | 100 | 206 |
| benzofuran-2-yl-[2- (2,4-dimethoxy- phenyl)-acetyl]- carbamic acid ethyl ester | I3 | 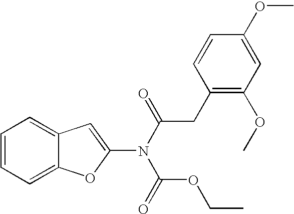 | F | ND | 100 | 384 |
| 5-chloro-benzofuran- 2-carboxylic acid 4- methoxy-benzylamide | I4 | 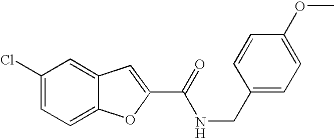 | B | 2.43 | 100 | 316 |
| 5-chloro-benzofuran- 2-carboxylic acid 4- hydroxy-benzylamide | I5 | 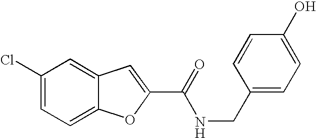 | I | 2.07 | 100 | 302 |
| 4-aminomethyl-N,N- dimethyl- benzenesulfonamide | I6 | 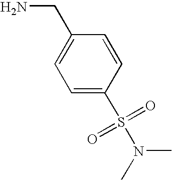 | M | 1.01 | 100 | 251 |
| 3-acetylamino-5- chloro-benzofuran-2- carboxylic acid methyl ester | I7 | 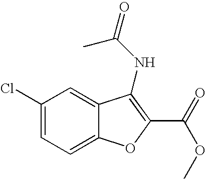 | N | 2.04 | 95 | 268 |
| 5-chloro-1-methyl-1H- indole-2-carboxylic acid | I8 | 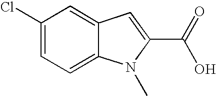 | C | 2.16 | 100 | 210 |
| 3-acetylamino-5- chloro-benzofuran-2- carboxylic acid | | | | | | |

### Example 2

### Non-Limiting Examples of Compounds According to the Invention

The present invention encompasses compounds of Formula I to LVII as well as stereoisomers, tautomers, racemics, prodrugs, metabolites thereof, or a pharmaceutically acceptable salt and/or solvate thereof.

Compound 1: 5-chlorobenzofuran-2-carboxylic acid (R)-[(4-nitrophen-1-yl)ethyl]-amide

This compound was obtained from 5-chlorobenzofuran-2-carboxylic acid and (R)-(4-nitrophen-1-yl)ethylamine, according to the protocol A.

Compound 2: 5-chlorobenzofuran-2-carboxylic acid 3,5-dimethoxybenzyl-amide

This compound was obtained from 5-chlorobenzofuran-2-carboxylic acid and 3,5-dimethoxybenzylamine, according to the protocol A.

Compound 3: 5-chlorobenzofuran-2-carboxylic acid 2-(5-methylindol-3-yl)-ethyl-amide

This compound was obtained from 5-chlorobenzofuran-2-carboxylic acid and 2-(5-methylindol-3-yl)ethyl amine, according to the protocol A.

Compound 4: 5-chlorobenzofuran-2-carboxylic acid [3-(10,11-dihydro-dibenzo[b,f]azepin-5-yl)propyl]methyl-amide

This compound was obtained from 5-chlorobenzofuran-2-carboxylic acid and [3-(10,11-dihydro-dibenzo[b,f]azepine-5-yl)propyl]methyl-amine, according to the protocol A.

Compound 5: 5-chlorobenzofuran-2-carboxylic acid [3-(10,11-dihydro-dibenzo[a,d]cycloheptene-5-ylidene)ethyl]-methyl-amide

This compound was obtained from 5-chlorobenzofuran-2-carboxylic acid and [3-(10,11-dihydro-dibenzo[a,d]cycloheptene-5-ylidene)propyl]methyl-amine, according to the protocol A.

Compound 6: 5-chlorobenzofuran-2-carboxylic acid (4-nitrobenzyl)-propyl-amide

This compound was obtained from 5-chlorobenzofuran-2-carboxylic acid and (4-nitrobenzyl)-propyl-amine, according to the protocol A.

Compound 7: 5-chlorobenzofuran-2-yl-[4-(4-chlorobenzoyl)-piperidin-1-yl]-methanone

This compound was obtained from 5-chlorobenzofuran-2-carboxylic acid and 4-(4-chlorobenzoyl)-piperidine, according to the protocol A.

Compound 8: 5-chlorobenzofuran-2-carboxylic acid 4-dimethylamino-benzylamide

This compound was obtained from 5-chlorobenzofuran-2-carboxylic acid and 4-dimethylamino-benzylamine, according to the protocol A.

Compound 9: 7-methoxybenzofuran-2-carboxylic acid (R)-(4-nitrophen-1-yl)-ethyl-amide

This compound was obtained from 7-methoxybenzofuran-2-carboxylic acid and (R)-(4-nitrophen-1-yl)-ethyl-amine, according to the protocol A.

Compound 10: 7-methoxybenzofuran-2-carboxylic acid (S)-(napht-2-yl)-ethyl-amide

This compound was obtained from 7-methoxybenzofuran-2-carboxylic acid and (S)-(napht-2-yl)-ethyl-amine, according to the protocol A.

Compound 11: 7-methoxybenzofuran-2-carboxylic acid (1R,2R)-2-(benzyloxy cyclopent-1-yl)-amide

This compound was obtained from 7-methoxybenzofuran-2-carboxylic acid and (1R,2R)-2-(5-benzyloxycyclopent-1-yl)-amine, according to the protocol A.

The compounds according to the invention are listed under Table 12. The invention encompasses the compounds 1 to 120 as listed in Table 12 as well as stereoisomers, tautomers, racemics, prodrugs, metabolites thereof, or a pharmaceutically acceptable salt and/or solvate thereof.

### Example 3

### Biological Assays Using C. elegans Screening

A C. elegans based high-throughput screen for Kv4.3 modulators has been used to establish an in vivo SAR (structure-activity relationships: the effect of chemical structure on biological activity) on Kv4.3 for the compounds according to the present invention.

This assay has employed a stable transgenic C. elegans strain that functionally expressed human Kv4.3 in the pharynx and a visible selection GFP maker in body-wall muscle.

The method to describe the construction of a transgenic C. elegans strain expressing human Kv4.3 has been described in WO 03/097682. Briefly, the actual used strain UG1755 has been generated by microinjection into the gonad of a wild-type strain N2 with a mix of 5 ng/μl plasmid pGV8 (human Kv4.3), 20 ng/μl pDW2821 (GFP-marker) and 40 ng/μl genomic C. elegans DNA. Transgenic animals have been isolated and submitted to integration of the extra-chromosomal array into the genome of C. elegans. A line with 50% transmission of the functional expressed human Kv4.3 has been mutagenized with gamma irradiation using a Cobalt source. About 12000 F2 animals have been singled out and their progeny has been screened for the 100% transmission of the GFP marker. Lines with 100% transmission of GFP have been considered as potentially integrated. These lines have been further tested and out-crossed with N2 strain two-times. All lines obtained have been tested for viability, GFP and human Kv4.3 expression. At the end of a selection process, UG1755 was identified as most suitable C. elegans strain amenable to high throughput screening (HTS). This stable strain has expressed human Kv4.3 as confirmed by electropharyngeograms (EPG) analysis. The method has been described in WO 03/097682. Briefly, dissected pharynxes of UG1755 C. elegans animals have been prepared and Electropharyngeograms (EPGs) have been recorded using an Axopatch-1D amplifier (Axon instruments). Pharynxes have been equilibrated for about 2 minutes in the bath solution (Dent's saline with 0.5% DMSO) until stable EPG recordings have been seen. Compound solution (DMSO or 100 μM Flecamide) has been added to the bath solution. The number of ultra short EPGs (10-20 ms) and normal EPGs (100-200 ms) has been analyzed and given as ratio in percentage. Reduction of the number of ultra short EPGs indicates partial reversion to wild-type EPGs and consequently inhibition of human Kv4.3. 50-80% of the EPGs are the ultra short EPGs (Table 2). Human Kv4.3 has induced ultra short EPGs in UG1755 that can be modulated with Flecamide. In addition standard qRT-PCR confirmed the presence of the human Kv4.3 transgene in UG1755 as well as human Kv4.3 protein has been detected with the human Kv4.3 antibody P 0358 in the pharynx of UG1755 animals.

TABLE 2
Flecainide modulates human Kv4.3 activity
in transgenic C. elegans strain UG1755.

| | Before Application | After Application |
|---|---|---|
| | of Compound Ratio | of Compound Ratio |
| | Ultra short to | Ultra short to |
| Strain | Normal EPGs in % | Normal EPGs in % |
| UG1755: DMSO (n = 10) | 76 ± 19 | 90 ± 5 |
| UG1755: Flecainide | 83 ± 13 | 53 ± 24 |
| (n = 15) | | |

Background of the Assay:

The pharynx is the feeding organ of C. elegans and contracts rhythmically 3-4 times per second. The pharynx contraction is controlled by the nervous system via action potentials similar to the human myocyte and can thus be used to study human ion channel physiology in vivo in C. elegans.

Introducing the human Kv4.3 channel into the C. elegans pharynx influences the C. elegans action potential in a characteristic fashion. The additional number of potassium channels increases potassium ion efflux, enhances re-polarization and thereby shortens the action potential duration. The ultra short action potentials of the human Kv4.3 transgenic C. elegans pharynx can be restored to normal action potentials with 4-aminopyridine, a non-specific potassium channel blocker, or flecamide, a SCN5a and Kv4.3 blocker. The shortened action potentials of the C. elegans pharynx resulting from expression of human Kv4.3, changes the pharynx contraction-relaxation cycle and consequently reduces the pumping/feeding in these transgenic animals. This change of pumping is an end-point amenable to high-throughput screening technology. The pumping end-point can be technically translated into a high-throughput read-out by the use of a pro-fluorescent dye. The pro-fluorescent dye is taken up by C. elegans depending on its pumping activity and converted into a fluorescent dye by enzymes located in its intestines. After a defined incubation period, the change of fluorescence intensity in the intestine can be measured with a plate reader.

The screening of the compounds according to the present invention has been performed in the C. elegans based high-throughput screen for Kv4.3 modulators described above. The method for testing the activity on human Kv4.3 of the compounds according to the invention with C. elegans strains expressing human Kv4.3 for their activity is the same as the method described in WO 03/097682. The method for testing the compounds on wild-type C. elegans strains not expressing human Kv4.3 is the same as the method described in WO 00/63427. Briefly, UG1755 animals have been grown in large numbers and staged young adults (no or only few eggs inside the uterus) have been harvested on the day of screening. Approximately 125 animals in 80 μl buffer have been dispensed per well of a “U” shaped 96-well compound plate. The compound plate contained already compound material at a final concentration of 30 μM in 0.3% DMSO. After one hour incubation 10 μl of the fluorescent label Calcein AM (CAM) have been added to achieve a final concentration of 5 μM CAM and 0.8% DMSO. After another four hours of incubation at 20° C., the drinking of C. elegans animals (or the “reaction” as measured by the uptake of CAM) has been stopped by adding 10 μl of a 60 μM ivermectin solution. The fluorescence intensity (counts per second) has been measured 40 minutes after adding ivermectin with the Wallac plate reader at a wavelength of 535 nm (after excitation at 485 nm).

The active compounds have been identified and confirmed by dose-response analysis. An EC₅₀ has been calculated and the results are listed under Table 3. Dose-response curves have been obtained at concentrations of 30 μM. EC₅₀ has been calculated using XLfit 2.09 software package.

These compounds have been also tested in the same assay format using a C. elegans wild-type strain N2 and the corresponding EC₅₀ has been calculated. The ratio of the EC₅₀s obtained for a compound on the two strains (transgenic expressing human Kv4.3 and wild-type) gives an indication on whether said compound is acting on human Kv4.3. The cut-off value to determine that a compound is potentially active on Kv4.3 was a ratio of 1.8 (EC₅₀ on N2 divided by EC₅₀ on the Kv4.3 expressing strain). The results of the ratio for the compounds according to the invention are listed under Table 3.

TABLE 3

| | EC₅₀ (μM) | EC₅₀ (μM) | Ratio |
|---|---|---|---|
| Compound | Kv4.3 worm | N2 worm | N2/Kv4.3 |
| 1 | 6.1 | >30 | >4.9 |
| 5 | 4.2 | 24 | 5.7 |

All the compounds tested were active on Kv4.3 at very low concentration. The compounds of the invention were also active on Kv1.5 ion channels as illustrated further in Example 5.

### Example 4

### Patch Clamp Assays

Cell Culture:

For this experiment, a recombinant CHO-K1 cell line stably expressing the human Kv4.3/KChIP2.2 potassium channel was used. The cells used for this experiment were kept in continuous culture under standard conditions (37° C., air supplemented with 7% CO₂). The CHO-K1 Kv4.3/KChIP2.2 cells were kept in Iscove's modified DMEM (Dulbecco's Modified Eagle's Medium) medium (IMEM) supplemented with 100 U/ml Penicillin, 100 μg/ml Streptomycin, 7% fetal calf serum (FCS), 2.5 μg/ml amphotericin, 400 μg/ml G418, and 400 μg/ml Zeocin™. Cells were passed every 3-4 days after detachment using a Trypsin solution. The quality of the cultured cells was guaranteed by vitality and contamination tests. The culture of the cells was performed as described in protocol B hereunder.

Protocol B: The cells were cultured in 94 mm culture dishes under the culturing conditions of 5% CO₂ and 37° C. Subculturing was performed every 3-4 days, by removing the media and then rising the dish with 8 ml PBS (phosphate buffered saline). The PBS was removed and 1 ml Trypsin/EDTA was added to the cells. The cells were incubated about 2 min at 37° C. or 5 min at room temperature and then the dish was rapped to detach and singularize the cells. To inactivate the enzyme 9 ml of media was added and the solution was pipetted up and down to break up clumps of cells. Part of the suspension was then transferred to a new 94 mm dish and media was added to a final volume of 8 ml. If necessary the cells could be seeded onto 35 mm or 94 mm dishes (2 ml media per 35 mm dish and 8 ml media per 94 mm dish). The media was changed every 2-3 days. The media used was the solution for culturing the cells described above. For stable cells antibiotic G418, Hygromycin, Blasticidin, or Zeocin were not added.

The PBS used was Dulbecco's PBS (1×), without Ca and Mg. The 10× Trypsin/EDTA solution contained 5 g/l Trypsin, 2 g/l EDTA and 8.5 g/l NaCl. The 1× Trypsin/EDTA was prepared by adding 450 ml PBS to 50 ml 10× Trypsin/EDTA.

At least 18 hours prior to electrophysiological experiments the cells were detached by application of iced PBS or Trypsin and replated on cover slips.

The preparation of cells for electrophysiological experiments was performed as described in protocol C hereunder.

Protocol C: The transfected cells and stable cells were transferred from 35 mm cell culture dishes onto coverslips using cold PBS: The media was removed and 0.3 ml of PBS (4-10° C.) was added. The cells were incubated 5 min at room temperature. The dish was rapped to detach and singularize the cells and 1.7 ml media was added and the solution pipetted up and down to break up clumps of cells. Part of the cell suspension was then transferred to a 35 mm dish with coverslips and media. The transfected cells and stable cells could also be transferred from 35 mm cell culture dishes onto coverslips using trypsin: The media was removed and the dish rinsed with 3 ml PBS. The PBS was removed and 0.3 ml 1× Trypsin/EDTA was added and the cells incubated 5 min at room temperature. The dish was rapped to detach and singularize the cells and 1.7 ml media was added and the solution pipetted up and down to break up clumps of cells. Part of the cell suspension was then transferred to a 35 mm dish with coverslips and media.

Preparation of Solutions:

10 mM stock solutions of the compounds were prepared in DMSO. Solutions of the compounds were prepared by dilution of stock solution in bath solution. If the required stock solution volume was theoretically below 1 μl, bath solutions with higher compound concentration were used for dilution. The maximal DMSO concentration during experiments was 0.1% (v/v).

For patch clamp experiments, the following solutions in demineralized water as vehicle were used (concentration in mM).

Bath (external) solution: 4 KCl, 135 NaCl, 2 CaCl₂, 1 MgCl₂, 10 D(+)-Glucose, 5 HEPES, pH 7.4 (NaOH).

Pipette (internal) solution: 130 KCl, 1 MgCl₂, 10 EGTA, 5 Na₂ATP, 5 HEPES, pH 7.4 (KOH).

Electrophysiological Measurements:

Activity of the human Kv4.3/KChIP2.2 channel was investigated using the patch clamp technique in its whole cell mode. This means that the current needed for clamping the whole cell expressing the K⁺-channel protein to a specific potential was measured. Experiments were performed using a patch clamp set-up. Technical equipment needed for manipulation of the cells was placed on a vibration-isolated table and shielded with a Faraday cage to minimize electrical noise. The amplifier and control system were placed in a rack outside the Faraday cage. The system consisted of an EPC9 or EPC10 patch clamp amplifier (HEKA, Lambrecht, Germany), and the perfusion system DADVC8 (ALA Scientific, New York, USA) controlled by the Pulse software package (HEKA, Lambrecht, Germany) installed on a personal computer. The pipettes used for patch clamping were made of borosilicate glass.

Measurements were performed at room temperature (20-25° C.). In each experiment (i.e. each cell) only one concentration of the compounds was investigated—no cumulative dosages were performed. Each cell acted as its own control. The effect of compounds on Kv4.3/KChIP2.2 mediated currents was investigated at one concentration (2 μM) with two replicates each (c=1, n=2).

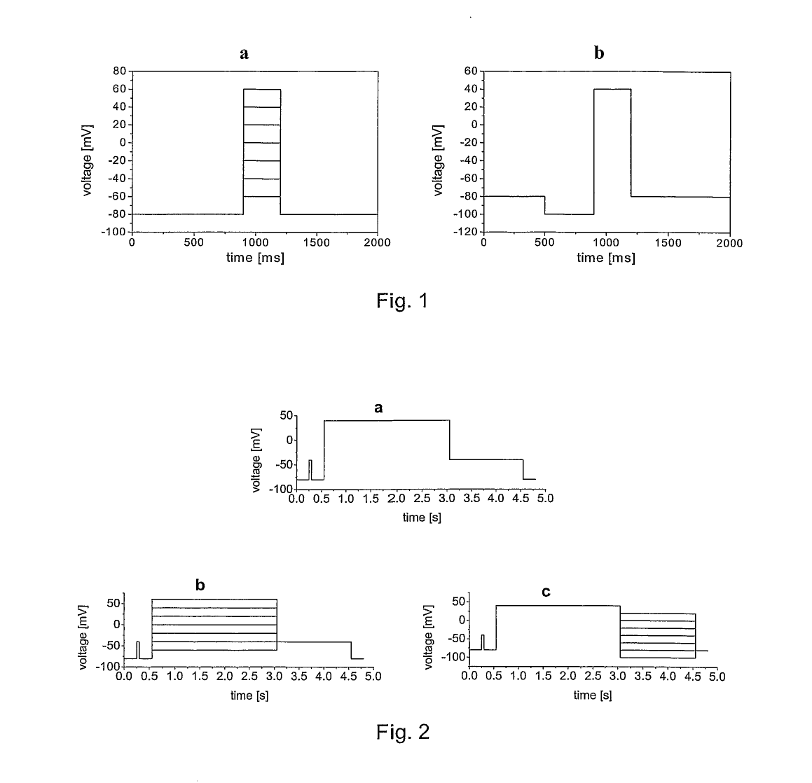

Between voltage pulses, the cell was clamped to a holding potential of −80 mV (inside). The test protocols are illustrated in FIG. 1 and Table 4. The test protocol illustrated in FIG. 1a was used to characterize the properties of the Kv4.3/KChIP2.2 channel and to check the quality of the individual patch clamp experiment (voltage control). The test protocol illustrated in FIG. 1b shows the standard test pulse for determination of channel activity. Each test protocol consisted of 4 segments. Duration and voltage of the segments are listed in Table 4.

TABLE 4
Test protocols for electrophysiological investigation
of human Kv4.3/KChIP2.2 channels

| | | |
|---|---|---|
| Protocol/segment | Duration (ms) | Voltage (mV) |

a) IV activation (7 pulse protocols)- FIG. 1a

| | | |
|---|---|---|
| Holding potential 1 | 500 | −80 |
| Holding potential 2 | 400 | −80 |
| Activation (varies with each pulse) | 300 | −60/−40/−20/0/ |
| | | 20/40/60 |
| Holding potential 2 | 800 | −80 |

b) Test pulse- FIG. 1b

| | | |
|---|---|---|
| Holding potential 1 | 500 | −80 |
| Full activation | 400 | −100 |
| Activation (varies with each pulse) | 300 | 40 |
| Holding potential 2 | 800 | −80 |

After the whole cell configuration was established a current-voltage curve (IV activation 1; FIG. 1a and Table 4a) was recorded under constant superfusion with bath solution to check for Kv4.3/KChIP2.2 expression and the quality of the individual patch clamp experiment (voltage control). Subsequently, 14 test pulses (series 1; FIG. 1b and Table 4b) were applied at 0.1 Hz under constant superfusion with bath solution. The protocol was initiated by a voltage jump to −100 mV (400 ms) to achieve full inactivation. Then the Kv4.3/KChIP2.2 channels were transferred to the open state by depolarization to +40 mV for 300 milliseconds (activation). Only cells generating current amplitudes between 1 nA and 50 nA were used for the experimental procedure. No or negligible rundown was observed.

Then, 36 test pulses (series 2) were applied at 0.1 Hz under constant superfusion with the compound dissolved in bath solution. Finally, another current-voltage curve was recorded in the presence of the compounds (IV activation 2).

If the experimental parameters were still satisfying, the experiment was extended to study the washout of the compound. To do this, the cell was again superfused with bath solution while up to 30 test pulses were applied at 0.1 Hz (series 3). The washout procedure was not necessary for correct data analysis.

All data were leak corrected using a P/n protocol with n=5 (see data analysis).

All test protocols/series and their use in data analysis are summarized in Table 5.

TABLE 5
Test protocols/series and their use in the data
analysis of electrophysiological measurements

| Test protocols/series | Use for data analysis |
|---|---|
| IV activation 1 | Kv4.3/KChIP2.2 channel characterization |
| Series 1 (14× test pulse) | Determination of rundown, 100% control |
| Series 2 (36× test pulse) | Determination of compound effects on |
| | current |
| IV activation 2 | Determination of compound effects on |
| | IV curve |
| Series 3 (optional up to 30× | Determination of washout properties of |
| test pulse) | compound |

Vehicle controls were performed in separate experiments during the experiment, using the compound vehicle (DMSO) at the highest concentration (0.1%) applied in the experiment (n=2).

Data Analysis

A leak correction was performed using a classical P/n protocol. The leak pulses should not reach the activation level of the channels, and thus were scaled down to 1/n of the original pulse amplitude. Here, n=5 was used. The current responses of the cell to the leak pulses were then multiplied by n to calculate a theoretical passive response of the cell to the test sequence. This calculated curve was then subtracted from the real response, leaving only the active part of the response.

Three different types of analysis were used: Inhibition at the peak current, inhibition of translocated charge and inhibition 75 ms after activation at +40 mV (sustained current). The peak current/charge/current at 75 ms was determined using the online analysis tool of the HEKA pulse software package. The cursors were placed in a way that the peak current was enclosed, the whole segment “activation” (see Table 5) was selected or a cursor was placed at time 75 ms. The resulting current peak amplitudes/translocated charges/current amplitudes at 75 ms were exported as ASCII data file.

The resulting ASCII files were imported into the software package Prism (Graphpad Software, San Diego, USA) and further analyzed as described below. No rundown correction was necessary. The last 5 current peak amplitudes/translocated charges/current amplitudes at 75 ms before application of compound solution were averaged and were used as 100% activity value. The last 5 current peak amplitudes/translocated charges/current amplitudes at 75 ms in presence of compound solution were averaged to give the inhibition value.

All data points were fitted with a Hill function with three independent parameters, wherein yₘₐₓ is the maximum inhibition in %, IC₅₀ is the concentration at half maximum inhibition and hill is the Hill coefficient.

$$y = \frac{{y}_{max}}{1 + {\{ \frac{{IC}_{50}}{x} \}}^{hill}}$$

The fit parameters yₘₐₓ, IC₅₀ and hill characterize the interaction of Kv4.3/KChIP2.2-channels expressed in CHO-K1 cells with the compound tested. The resulting curve fitting is displayed in a graph (% inhibition vs. log concentration) with the averaged results with error bars: Standard Error of the Means (SE M) wherein

$$SEM = \sqrt{\frac{\sum {x}^{2} - {( \sum x )}^{2} / n}{n ( n - 1 )}}$$

In order to compare the results, IC₅₀ values were estimated. Therefore, data were fitted using the Hill equation with fixed values for the Hill coefficient (hill=1) and the maximum inhibition at high concentrations (yₘₐₓ=100).

The results of the Patch Clamp experiments are shown in Table 6.

TABLE 6

| Compounds | IC₅₀ (μM) Kv4.3 |
|---|---|
| 1 | 2.08 |
| 2 | 1.75 |
| 3 | 1.33 |
| 4 | 3.81 |
| 5 | 16.02 |
| 7 | 0.89 |

The compounds according to the invention were found to be particularly active against Kv4.3 ion channels.

In order to be maximally useful in treatment, it was also important to assess the side reactions which might occur. Thus, in addition to being able to modulate a particular calcium channel, it was shown that the compounds according to the invention had high selectivity for Kv4.3 versus the hERG channel.

Test System and Test Method for the hERG Experiment

Test System:

For this experiment HEK 293 T-REx HERG cells (#23) were used. This cell line made by IonGate is characterized by the inducible expression of the hERG gene. The T-REx™ System (Invitrogen, Karlsruhe, Germany) is a tetracycline-regulated mammalian expression system that uses regulatory elements from the E. coli Tn10-encoded tetracycline (Tet) resistance operon. In the absence of Tet the expression is repressed. Tetracycline regulation in the T-REx™ System is based on the binding of tetracycline to the Tet repressor and derepression of the promoter controlling expression of the hERG gene. Addition of Tet to the cell culture media results in expression of the hERG potassium channel.

To construct the HEK 293 T-REx HERG cell line, the hERG gene was ligated into the inducible expression vector pcDNA4/TO (→pc4TO-HERG) and transfected into HEK 293 T-REx cells (this cell line stably expresses the Tet repressor and was purchased at Invitrogen). Stable cell clones were isolated after selection with Blasticidin (5 μg/ml) and Zeocin™ (300 μg/ml). The clones were electrophysiological characterized after induction with 1 μg/ml Tet. Clone #23 showed the best expression of the hERG potassium channel.

Test Method

Experiments were performed using the patch clamp set-up. Technical equipment needed for manipulation of the cells was placed on a vibration-isolated table and shielded with a Faraday cage to minimize electrical noise. The amplifier and control system were placed in a rack outside the Faraday cage. The system consisted of an EPC9 or EPC10 patch clamp amplifier (HEKA, Lambrecht, Germany), and the perfusion system DADVC8 (ALA Scientific, New York, USA) controlled by the Pulse software package (HEKA, Lambrecht, Germany) installed on a personal computer.

Cell Culture

The cells used for this experiment were kept in continuous culture under standard conditions (37° C., air supplemented with 5% CO₂). The HEK 293 T-REx HERG cells were kept in minimal essential medium (MEM) supplemented with 100 U/ml Penicillin, 100 μg/ml Streptomycin, 10% fetal calf serum (FCS), 1% non-essential amino acids (NEAA), 2.5 μg/ml amphotericin, 300 μg/ml Zeocin™ and 5 μg/ml Blasticidin. Cells were passed every 3-4 days after detachment using a Trypsin solution. The quality of the cultured cells was guaranteed by vitality and contamination tests. The culture of the cells was performed as described in protocol B described above.

At least 18 hours prior to electrophysiological experiments the cells were detached by application of iced PBS (phosphate buffered saline) or Trypsin and replated on cover slips. 1 μg/ml Tet was added to the cells to induce hERG expression.

The preparation of cells for electrophysiological experiments was performed according to protocol C described above. The solutions were prepared as described above under in the paragraph preparation of solutions.

Electrophysiological Measurements:

Activity of the cardiac hERG channel was investigated using the patch clamp technique in its whole cell mode. This means that the current needed for clamping the whole cell expressing the K⁺-channel protein to a specific potential was measured. The pipettes used for patch clamping were made of borosilicate glass. Measurements were performed at room temperature (20-25° C.). In each experiment (i.e. each cell) only one concentration of the compound was investigated—no cumulative dosages were performed. Each cell acted as its own control. The effect of the compounds on hERG mediated currents was investigated at one concentration (10 μM) with two replicates each (c=1, n=2).

Between voltage pulses the cell was clamped to a holding potential of −80 mV (inside).

The test protocols for electrophysiological investigation of hERG K⁺-channels are illustrated in FIG. 2 and Table 7. In Table 7 and FIG. 2, (a) is the standard test pulse for determination of channel activity. (b) and (c) were used to characterize the properties of the hERG channel and to check the quality of the individual patch clamp experiment (voltage control). Each test protocol consisted of 6 segments. The duration and voltage of the segments are listed in Table 7.

TABLE 7

| | | |
|---|---|---|
| Protocol/segment | Duration (sec) | Voltage (mV) |

a) Test pulse - FIG. 2a

| | | |
|---|---|---|
| Holding potential 1 | 0.25 | −80 |
| Leak-test | 0.05 | −40 |
| Holding potential 2 | 0.25 | −80 |
| Activation | 2.5 | 40 |
| Tail-current test | 1.5 | −40 |
| Holding potential 3 | 0.25 | −80 |

b) IV activation (7 pulse protocols) - FIG. 2b

| | | |
|---|---|---|
| Holding potential 1 | 0.25 | −80 |
| Leak-test | 0.05 | −40 |
| Holding potential 2 | 0.25 | −80 |
| Activation (varies with each pulse) | 2.5 | −60/−40/−20/0/ |
| | | 20/40/60 |
| Tail-current test | 1.5 | −40 |
| Holding potential 3 | 0.25 | −80 |

c) IV tail current (7 pulse protocols) - FIG. 2c

| | | |
|---|---|---|
| Holding potential 1 | 0.25 | −80 |
| Leak-test | 0.05 | −40 |
| Holding potential 2 | 0.25 | −80 |
| Activation | 2.5 | 40 |
| Tail-current test (varies with each | 1.5 | −100/−80/−60/−40/ |
| pulse) | | −20/0/20 |
| Holding potential 3 | 0.25 | −80 |

After the whole cell configuration was established, a series of pulses was applied to check for hERG expression and to optimize amplifier settings. However, only the following measurements were used for data analysis: 15 test pulses (FIG. 2a and Table 7a) were applied at 0.1 Hz (series 1) under constant superfusion with bath solution. The protocol was initiated by a leak test at −40 mV (50 ms). After returning to the holding potential (−80 mV, 0.25 sec.) hERG channels were transferred to the inactive state by depolarisation to +40 mV for 2.5 seconds (activation). The maximum hERG tail current amplitude was measured at −40 mV (tail current test, 1.5 sec.). Only cells generating tail current amplitudes between 300 pA and 10 nA were used for the experimental procedure.

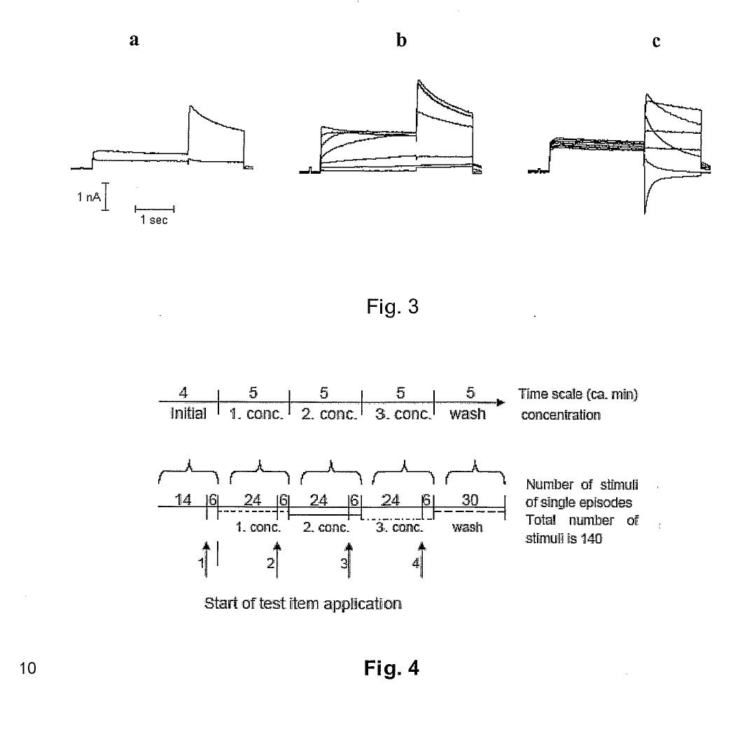

The hERG channel mediated currents evoked by test protocols for hERG channel testing in FIG. 3. FIG. 3a shows the test of channel activity with and without 10 μM of a test compound (upper trace). FIGS. 3b and 3c are the current response to protocol Table 7b (IV activation) and Table 7c (IV tail current).

Subsequently the current-voltage curve (IV-curve) was investigated using two pulse series under superfusion with bath solution. The pulse series “IV activation” varies the potential of the activating pulse between each consecutive pulse of the series (−60 to +60 mV in 20 mV intervals, FIG. 2b and Table 6b). The tail current amplitude after activation at +60 mV had to be within ±20% of the value found with +40 mV activation potential. The pulse series “IV tail current” varies the potential of the tail current test pulse between each consecutive pulse of the series (−100 to +20 mV in 20 mV intervals, FIG. 2c and Table 6c). The maximum tail current amplitude (Iₘₐₓ) had to be measured at −40 mV (±10% Iₘₐₓ). Test pulses for series “IV activation” and “IV tail current” were applied at 0.1 Hz.

After successful characterization of the current another 10 test pulses were applied at 0.1 Hz while the cell was superfused with bath solution (series 2). Series 1 and 2 were used to fit a mathematical function to the tail current peak values to determine the rundown of the signal amplitude. Another 30 test pulses (series 3) were applied while the cell was superfused with a solution containing the compounds in the desired concentration. More test pulses were applied if necessary (series 4).

If the experimental parameters were still satisfying, the experiment was extended to study the washout of the compound. To do this, the cell was again superfused with bath solution while up to 20 test pulses were applied at 0.1 Hz (series 5). However, since the baseline was constructed by a fitting procedure to series 1 and 2 (rundown-correction) the washout procedure was not necessary for correct data analysis.

All test protocols/series and their use in data analysis of electrophysiological measurements are summarized in Table 8.

TABLE 8

| Test-protocols/series | Use for data analysis |
|---|---|
| Series 1 (15× test pulse) | Determination of rundown |
| IV activation | hERG channel characterization |
| IV tail-current | hERG channel characterization |
| Series 2 (10× test pulse) | Determination of rundown, 100% control |
| Series 3 (up to 30× test pulse) | Determination of compound effects on |
| | tail current |
| Series 4 (optional - up to 20× | Determination of compound effects on |
| test pulse) | tail current |
| Series 5 (optional - up to 20× | Determination of washout properties of |
| test pulse) | the compound |

Vehicle controls were performed in separate experiments during the analysis, using the compound vehicle (DMSO) at the highest concentration (0.1%) applied in the experiment (n=2).

Data Analysis

The data analysis was based on the tail current peak amplitude mediated by hERG K⁺ channels at −40 mV after activation at +40 mV. The peak current was determined using the online analysis tool of the HEKA pulse software package. The cursors were placed in a way that the peak current was enclosed. The current found at the leak test pulse (segment 2 in each test pulse) was set to zero. The resulting tail current peak amplitudes were exported as ASCII data file.

The resulting ASCII files were imported into the software package Prism (Graphpad Software, San Diego, USA) and further analyzed as described below. Series 1 and 2 were fitted using a suitable function (i.e. mono- or biexponential decay). The resulting function was used for rundown correction of the data set (series 1 to 5) by division. The last 5 tail current peak amplitudes before application of compound solution were averaged and were used as 100% activity value. The last 5 tail current peak amplitudes in presence of compound solution were averaged to give the inhibition value. All data points were fitted with a Hill function with three independent parameters, wherein yₘₐₓ is the maximum inhibition in %, IC₅₀ is the concentration at half maximum inhibition and hill is the Hill coefficient.

$$y = \frac{{y}_{max}}{1 + {\{ \frac{{IC}_{50}}{x} \}}^{hill}}$$

The fit parameters yₘₐₓ, IC₅₀ and hill characterize the interaction of hERG K⁺-channels expressed in HEK 293 cells with the compound tested. The resulting curve fitting is displayed in a graph (% inhibition vs. log concentration) with the averaged results with error bars: Standard Error of the Means (SEM) wherein

$$SEM = \sqrt{\frac{\sum {x}^{2} - {( \sum x )}^{2} / n}{n ( n - 1 )}}$$

In order to compare the results, IC₅₀ values were estimated. Therefore, data were fitted using the Hill equation with fixed values for the Hill coefficient (hill=1) and the maximum inhibition at high concentrations (yₘₐₓ=100).

The results showing the channel activity are illustrated in Table 9.

TABLE 9

| | IC₅₀ (μM) | IC₅₀ (μM) | Ratio |
|---|---|---|---|
| Compound | Kv4.3 | hERG | hERG/Kv4.3 |
| 2 | 1.75 | 16.74 | 9.6 |
| 4 | 3.81 | 20.03 | 5.2 |

The compounds showing a selectivity of 5 or >5 (ratio value) for Kv4.3 vs hERG (Patch Clamp test) were considered as being very selective toward Kv4.3 channels.

So in addition to being actives on Kv4.3 ions channels at very low concentration, the compounds according to the invention proved to be very selective toward Kv4.3 ions channels when compared to the hERG channel.

In addition, Table 12 shows the effects on Kv4.3 and hERG of a non-limiting number of additional compounds of the invention. Unless provided otherwise, the compounds were investigated at one concentration (1 μM) on the Kv4.3-mediated potassium channel, in a patch clamp assay following a protocol as described in Example 4. The results are shown in Table 12. In Table 12, Kv4.3 charge in %: means the remaining current measured after application of the compound and relative to the blank, and Kv4.3 peak in %: means the remaining peak height measured after application of the compound and relative to the blank. As used herein the term “ND” means not determined yet. Unless otherwise specified, the tests were performed at 1 μM for Kv4.3 charge and peak. The effects of a non-limiting number of additional compounds of the invention were investigated at 10 μm concentration unless provided otherwise on the hERG channel in patch clamp assay.

### Example 5

### Patch Clamp Assays Using the Kv1.5 Ion Channel

The cDNA coding for Kv1.5 (GenBank Acc. No. M55513) was cloned into the pcDNA6-vector (Invitrogen, Leek, Netherlands). A C-terminal epitope-tag was introduced via PCR. The plasmid was sequenced and subsequently introduced into cells. Clonal cell lines stably expressing the Kv1.5 channel were established. Expression of protein was analysed by means of immunofluorescence using antibodies directed against the epitope-tag. The functional expression of the ion channels was validated electrophysiologically.

Cell Culture

The experiments were performed using CHO cells stably expressing the Kv 1.5 potassium channel.

Cells were grown at 37° C. and 5% CO₂ in 25 ml flasks (Greiner Bioone, Cellstar, Cat. No. 690160) in 6 ml MEM ALPHA Medium (Sigma, Taufkirchen, Germany, Cat No M8042) supplemented with 10% (v/v) heat inactivated fetal calf serum (Sigma, Cat. No. F9665), 1% (v/v) P/S/G-solution (Sigma, Cat. No. G6784) and G-418 (750 μg per millilitre medium; Sigma, Germany, Cat. No. A1720; 50 mg/ml in water, Sigma, Germany, Cat. No. W3500).

Electrophysiology

Stimulation Protocol for the Kv1.5-Mediated Current

From a HP of −60 mV cells were hyperpolarised for 100 ms to −70 mV, followed by a 500 ms depolarisation to +50 mV. The current amplitude at the end of the test pulse to +50 mV was used for the analysis. Pulse cycling rate was 10 s (0.1 Hz).

Test Item Application Protocol for the Kv1.5 Mediated Current

The application protocol of test compounds is depicted in FIG. 4. The first 14 stimuli were required to achieve steady state of the current amplitude. Unspecific current reduction was calculated and served for correcting procedures during data analysis. After the 14ₜₕ stimulus the test compound application was started (indicated by an arrow) via teflon and silicone tubings and was assumed to reach the cell after 6 additional stimuli. The perfusion is adjusted by using a defined drop rate of 10 drops per 10-12 s. Up to three concentrations were applied successively to one cell followed by a wash period of 5 minutes. Total number of stimuli was 140. Effect of the test compound was analysed between stimulus nos. 21 and 50 (5 min., long dashed line) for the first concentration, between stimulus nos. 51 and 80 (5 min., short dashed line) for an additional 5 minutes. If the cell was still stable, a wash was added afterwards. Start of test compound application at the given concentration is indicated by arrows. Number of stimuli of each single episode are shown in the protocol of FIG. 4.

Negative Control

Vehicle (DMSO) control experiments were performed during study period and under identical conditions to verify the stability of current over time and to value cell condition.

Effects of Test Compounds on the Kv1.5 Mediated Current

The effects of the test compounds were investigated at one concentration (2 or 1 μM) on the Kv1.5-mediated potassium channel. For comparison, the vehicle control experiments on the Kv1.5 mediated potassium channel results are presented in Table 10.

TABLE 10

| | | Relative remaining | Relative remaining |
|---|---|---|---|
| | | current (mean after | current (mean after |
| Compound | Concentration | 5 min) | 8-10 min) |
| 2 | 2 μM | 0.88 (88%) | 0.66 (66%) |
| 89 | | ND | 82% |
| 107 | | ND | 89% |
| DMSO | 0.1% | 1.00 (100%) | 0.96 (96%) |

### Example 6

### Ex-Vivo Organ Studies in Rats and Guinea Pigs

The compounds were checked for their effect on the force of contraction, stimulation threshold and for the Functional Refractory Period (FRP) in isolated rat left atria (Rat LA). Rat left atria functionally express the Kv4.3 ion channel, producing the Iₜₒ current of the action potential. In addition, the compounds were checked for these respective effects in isolated guinea pig right ventricular papillary muscle (GP pap. muscle), which do not express Kv4.3. The guinea pig action potential is dominated by hERG like ion channels for the refractory currents. Consequently, activity of hERG channels in vivo should be seen in GP papillary muscle preparations.

Method: Rat LA (Same Method Applies to GP Pap. Muscle)

Assay Principle

Left atria were mounted vertically in a two-chambered organ bath containing 100 ml of buffer solution (in mM: NaH2PO4 0.6, MgSO4 0.6, KCl 4.7, NaHCO3 25, glucose 4.5, NaCl 120, CaCl2 2.4). The solution was saturated with and circulated by a gas mixture containing 95% O₂ and 5% CO₂. The temperature was kept constant at 30° C. Preload of the atria was set at about 8 mN. Electrical stimulation at 1 Hz was accomplished by rectangular pulses with a duration of 1.5 ms and a strength of 3.5× threshold. The isometric force of the preparations was measured by force transducers, connected to amplifiers, documented by a pen recorder and fed into a computer for evaluation. Force of contraction (FC), threshold stimulus (TS) and the functional refractory period (FRP) were measured at baseline (pre), 20 min after addition of compound, and after washout at the end of the experiment. Threshold stimulus, representing excitability of the tissue, was assessed by varying the voltage applied for electrical stimulation. TS was defined as the lowest voltage that induces a contraction of the tissue. The functional refractory period, representing the time needed for repolarization, was assessed by applying extra stimuli at varying time intervals from the preceding regular pacing stimulus. FRP was defined as the shortest interval between regular and extra stimulus that resulted in a contraction of the tissue in response to the extra stimulus.

Compound Application

Compounds were tested by five cumulative administrations at 20 minute intervals beginning after an equilibration period of at least 60 minutes. Two washout intervals followed the measurement at the highest concentration of the compound.

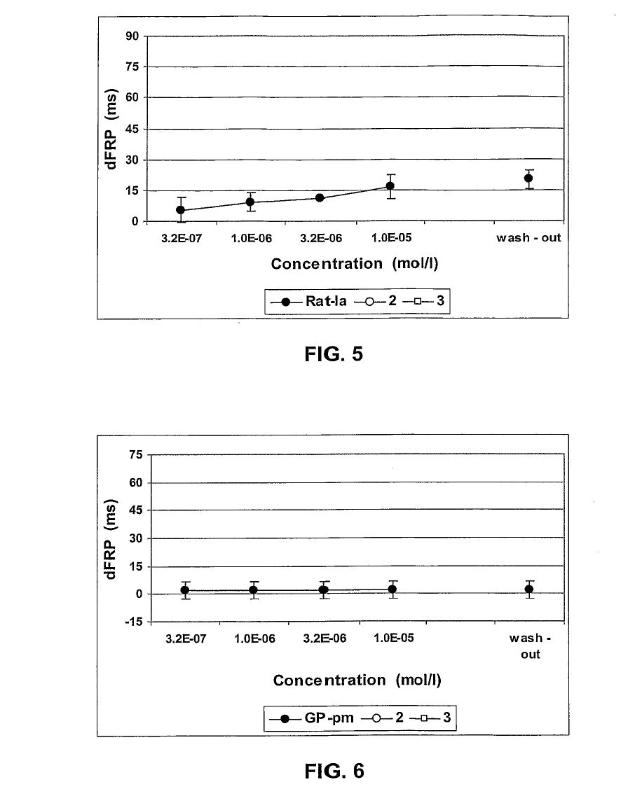

The results are depicted in FIGS. 5 and 6 and in Table 11.

FIG. 5 shows the functional refractory period in isolated rat left atria for compound 2.

FIG. 6 shows the functional refractory period in isolated guinea pig papillary muscle for the same compound.

TABLE 11

| | dFRP Rat | dFRP Guinea Pig |
|---|---|---|
| Compound | (conc 10⁻⁵ mol/l) | (conc 10⁻⁵ mol/l) |
| 2 | 17 ms | 0 ms |

### Example 7

### In Vivo Studies in Mice

Transmitter Implantation and ECG Recording

Male mice (NRMI) were anesthetized with a gas mixture of isoflurane, nitrous oxide and oxigen. Leads connected to a telemetry transmitter (TA10EA-F20, DSI, St. Paul, USA) were fixed by suture in the xiphoid and ventral neck region. The telemetry transmitter was placed under the skin on the back. Wounds were closed in layers and the animals were allowed to recover for at least 1 week.

Female guinea pigs (Charles River, Crl:HA(BR) were anesthetized by inhalative halothane anesthesia. The negative biopotential lead of the telemetry transmitter (TA11CTA-F40, DSI, St. Paul, USA) was fixed at muscle tissue in the right shoulder region, and the positive lead was fixed in the region of 6th left rib of the thorax, mimicking a standard lead II configuration. The telemetry transmitter was placed in the abdominal cavity, fixed to the peritoneal muscle, and the incision was sutured in layers. After transmitter implantation, the animals were allowed to recover for at least 1 week.

Experimental Study Design, Intraperitoneal (i.p.) Application

On the day of the experiment the animals receive consecutive doses of vehicle i.p. at 60 min dosing intervals. ECG tracings (12 s duration) was recorded using the Data Sciences A.R.T. system. After completion of the experiment ECGs were analyzed automatically by Data Sciences ECG software (DSI, St. Paul, USA). QT and QRS intervals were measured manually in the stored ECGs. QTc was calculated from the QT interval and the corresponding heart rate using Bazett's formula. Heart rate was taken from the online analysis, given by the DSI Labpro and DSI A.R.T. systems (DSI, St. Paul, USA). Finally, ECG intervals were transferred to an Excel spreadsheet, checked for plausibility, and converted into 15 min averages.

Results in Mice

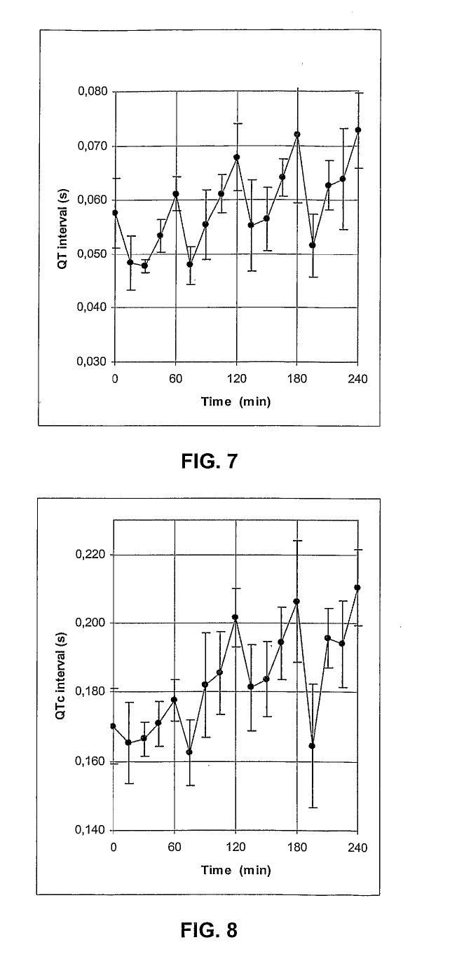

Consecutive dosing of the compounds of the invention led to a significant, dose-dependent but late onset prolongation of QT and QTc intervals in the mouse ECG. Results of vehicle and compound 2 are shown in FIGS. 7 and 8 respectively. The maximum prolongation of QT and QTc was achieved in the highest dose tested (15 μmol/kg) and amounted to 12 ms/33 ms, respectively. PQ and QRS did not show dose-dependent changes. Heart rate showed a minor and not significant decrease. Locomotor activity showed a reproducible rise after each of the subsequent injections. FIG. 7 shows the effects of vehicle and compound 2 (3-15 μmol/kg i.p) on QT interval in conscious telemetric mice. Means ±SD, n=5. FIG. 8 shows the effects of vehicle and compound 2 (3-15 μmol/kg i.p) on QTc interval in conscious telemetric mice. Means ±SD, n=5.

### CONCLUSIONS

The dose-dependent prolongation of QT and QTc which in mouse depends on Kv4.2 and Kv4.3, and the lack of effects on PQ and QRS seen after consecutive application of compounds indicate block of repolarizing K+ currents, which would be compatible with the compound's characterization as Kv4.2/Kv4.3 blocker. It should be noted that in this experiment the highest dose tested was 15 μmol/kg, higher doses of up to 30 μmol/kg may evenly be tested in this model.

TABLE 12
Effects of test compounds on the Kv4.3 meidated current
The following abbreviations are used hereunder. P: protocol, PU: retention time, PU: purity.
ESt: molecular ion obtained by electrospray in positive ion mode. m.p. = point.

| | Com- | | | | | | | Kv4.3 | Kv4.3 | |
|---|---|---|---|---|---|---|---|---|---|---|
| Name | pound | Structure | P | Rt | PU | ES⁺ | m.p. | charge | peak | hERG |
| 5-chloro-benzofuran-2- carboxylic acid [(R)-1-(4-nitro- phenyl)-ethyl]-amide | 1 | 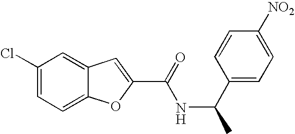 | A | 2.59 | 100 | 345- 347 | IC50 2.08 μM | ND | ND | |
| 5-chloro-benzofuran-2- carboxylic acid 2,4- dimethoxy- benzylamide | 2 | 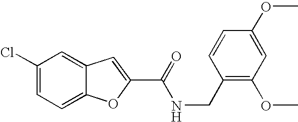 | A | 2.62 | 95 | 946- 348 | ND | IC50 1.75 μM | ND | IC50 16.74 μM |
| 5-chloro-benzofuran-2- carboxylic acid [2-(5- methyl-1H-indole-3-yl)- ethyl]-amide | 3 | 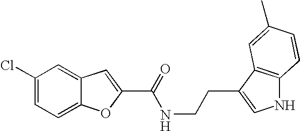 | A | 2.71 | 100 | 353- 355 | ND | IC50 1.33 μM | ND | ND |
| 5-chloro-benzofuran-2- carboxylic acid [3- (10,11-dihydro- dibenzo[b,f]azepin-5-yl)- propyl]-methyl-amide | 4 | 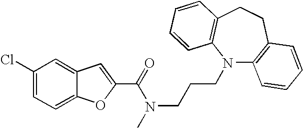 | A | 3.33 | 100 | 445- 447 | ND | IC50 3.81 μM | ND | IC50 20.03 μM |
| 5-chloro-benzofuran-2- carboxylic acid [3- (10,11-dihydro- dibenzo[a,d]cyclohepten- 5-ylidene)-propyl]- methyl-amide | 5 | 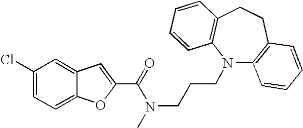 | A | 3.37 | 100 | 442- 444 | ND | IC50 16.02 μM | ND | ND |
| 5-chloro-benzofuran-2- carboxylic acid (4-nitro- benzyl)-propyl amide | 6 | 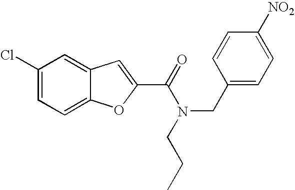 | A | 2.96 | 95 | 373- 375 | ND | ND | ND | ND |
| (5-chloro-benzofuran-2- yl)-[4-(4-chloro- benzoyl)-piperidin-1-yl]- methanone | 7 | 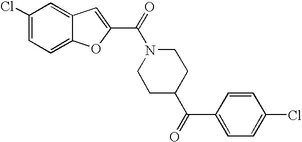 | A | 2.95 | 95 | 402- 404 | ND | IC50 0.89 μM | ND | ND |
| 5-chloro-benzofuran-2- carboxylic acid 4- dimethylamino- benzylamide | 8 | 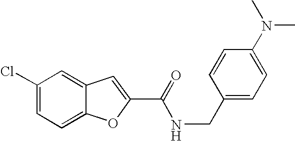 | A | 2.10 | 100 | 329- 331 | ND | ND | ND | ND |
| 7-methoxy-benzofuran- 2-carboxylic acid [(R)-1- (4-nitro-phenyl)-ethyl]- amide | 9 | 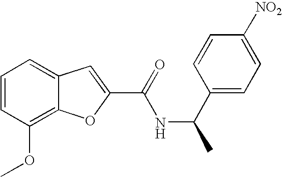 | A | 2.42 | 100 | 341 | ND | ND | ND | ND |
| 7-methoxy-benzofuran- 2-carboxylic acid ((S)-1- naphthalen-2-yl-ethyl)- amide | 10 | 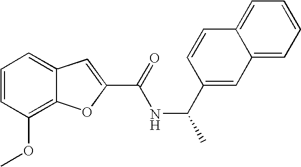 | A | 2.73 | 86 | 346 | ND | ND | ND | ND |
| 7-methoxy-benzofuran- 2-carboxylic acid ((1R,2R)-2-benzyloxy- cyclopent-1-yl)-amide | 11 | 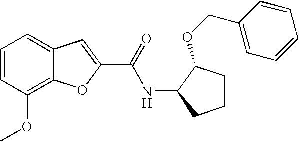 | A | 2.68 | 93 | 366 | ND | ND | ND | ND |
| benzofuran-2-carboxylic acid 2,4-dimethoxy- benzylamide | 12 | 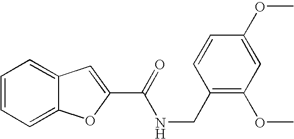 | B | 2.37 | 100 | 312 | 131.3 131.8 | 89% | 97% | ND |
| 4-acetyl-7-methoxy- benzofuran-2-carboxylic acid 2,4-dimethoxy- benzylamide | 13 | 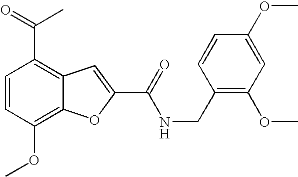 | A | 2.21 | 100 | 384 | 148.2- 150.3 | 86% | 99% | ND |
| 5-choro-3-methyl- benzofuran-2-carboxylic acid 2,4-dimethoxy- benzylamide | 16 | 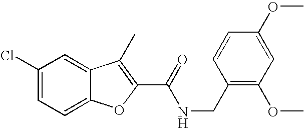 | B | 2.78 | 100 | 360 | 139.8- 140.4 | 24% | 88% | 47% |
| 3-pyrrol-1-yl-benzofuran- 2-carboxylic acid 2,4- dimethoxy-benzylamide | 18 | 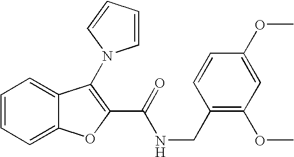 | B | 2.69 | 100 | 377 | 46.0- 51.8 | 45% | 88% | 41% |
| 5-chloro-1-methyl-1H- indole-2-carboxylic acid 2,4-dimethoxy- benzylamide | 19 | 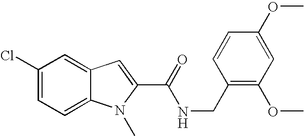 | A | 2.63 | 100 | 359 | 150.8- 152.7 | 44% | 87% | 35% |
| 5-chloro-2-methyl- benzo[b]thiophene-2- carboxylic acid 2,4- dimethoxy-benzylamide | 21 | 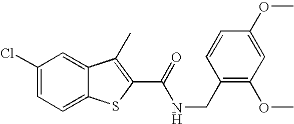 | B | 2.75 | 100 | 376 | 146.8- 147.8 | 50% | 92% | 100% |
| N-benzofuran-2-yl-2- (2,4-dimethoxy-phenyl)- acetamide | 22 | 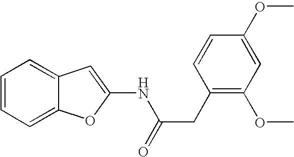 | G | 2.37 | 100 | 312 | 126.4- 128.2 | 90% | 100% | ND |
| 1-benzofuran-2-yl-2-(2,4- dimethoxy-phenyl)- ethanone | 23 | 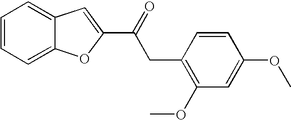 | H | 2.53 | 100 | 297 | ND | 83% | 99% | ND |
| 5-chloro-benzofuran-2- carboxylic acid (2,4- dimethoxy-phenyl)- amide | 24 | 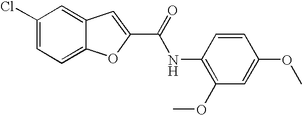 | B | 2.71 | 100 | 332 | 161.6- 163.3 | 85% | 102% | ND |
| 5-chloro-benzofuran-2- carboxylic acid indan-2- ylamide | 25 | 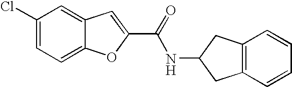 | A | 2.64 | 100 | 312 | 190.2- 190.5 | 82% | 100% | ND |
| (5-chloro-benzofuran-2- yl)-(1,3-dihydro-isoindol- 2-yl)-methanone | 26 | 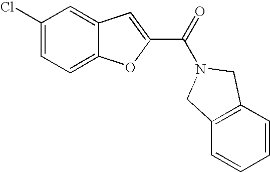 | B | 2.54 | 100 | 298 | 187.1- 188.5 | 81% | 99% | ND |
| (5-chloro-benzofuran-2- yl)-(3,4-dihydro-1H- isoquinolin-2-yl)- methanone | 27 | 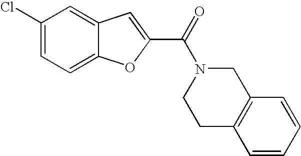 | B | 2.65 | 100 | 312 | 127.7- 129.1 | 59% | 86% | ND |
| (2-benzyl-piperidin-1-yl)- (5-chloro-benzofuran-2- yl)-methanone | 28 | 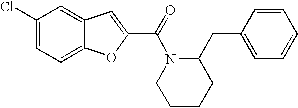 | B | 2.95 | 95 | 354 | — | 38% | 86% | 6% |
| benzo[b]thiophene-2- carboxylic acid 2,4- dimethoxy-benzylamide | 32 | 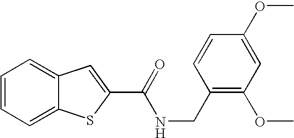 | B | 2.37 | 100 | 328 | 134.4- 135.3 | 75% | 100% | ND |
| acetic acid 4-{[(5-chloro- benzofuran-2-carbonyl)- amino]-methyl}-phenyl ester | 33 | 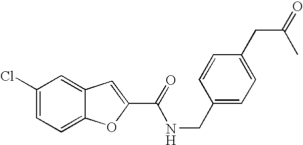 | J | 2.34 | 100 | 344 | 162.5- 164.5 | 89% | 100% | ND |
| 5-chloro-benzofuran-2- carboxylic acid 4- thiophen-2-yl- benzylamide | 34 | 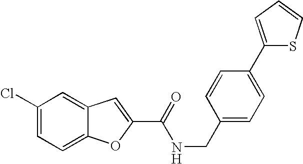 | B | 2.82 | 97 | 368 | 173.5- 175.0 | 90% | 98% | ND |
| 5-chloro-benzofuran-2- carboxylic acid 4-methyl- benzylamide | 36 | 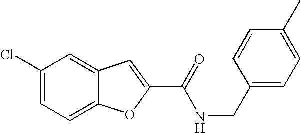 | B | 2.61 | 100 | 300 | 161.9- 162.9 | 72% | 89% | ND |
| (5-chloro-benzofuran-2- yl)-(4-phenyl-piperidin- 1-yl)-methanone | 38 | 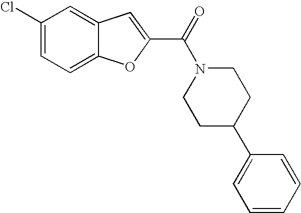 | B | 2.86 | 100 | 340 | 101.0- 101.6 | 89% | 99% | ND |
| 5-chloro-benzofuran-2- carboxylic acid 4- (thiophen-2-ylmethyl)- amide | 39 | 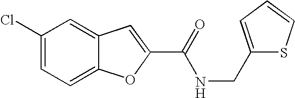 | B | 2.36 | 100 | 292 | 148.4- 149.9 | 90% | 91% | ND |
| (S)-[(5-chloro- benzofuran-2-carbonyl)- amino]-phenyl-acetic acid | 41 | 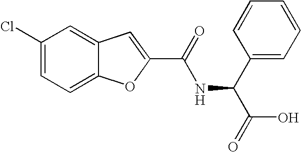 | K | 2.23 | 100 | 330 | 211.2- 215.6 | ND | ND | ND |
| quinolin-3-carboxylic acid 2,4-dimethoxy- benzylamide | 42 | 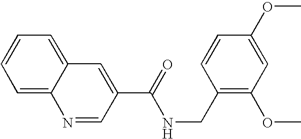 | B | 1.95 | 100 | 323 | 136.3 | 89% | 98% | ND |
| 3-methyl-benzofuran-2- carboxylic acid 2,4- dimethoxy-benzylamide | 43 | 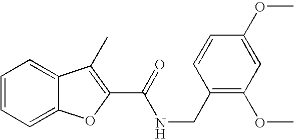 | B | 2.56 | 100 | 326 | 110.7- 111.5 | 70% | 94% | 61% |
| 5-chloro-benzofuran-2- carboxylic benzyl ester | 44 | 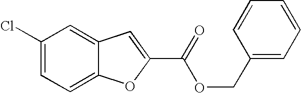 | L | 2.93 | 100 | 287 | 76.9- 77.3 | 45% | 80% | 28% |
| 5-chloro-benzofuran-2- carboxylic benzylamide | 47 | 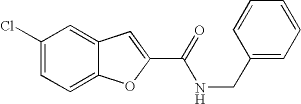 | B | ND | 100 | 286 | ND | 87% | 95% | ND |
| 5-chloro-benzofuran-2- carboxylic acid (2- dimethylamino-1-phenyl- ethyl)-amide | 49 | 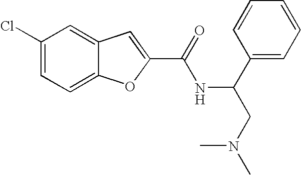 | B | 1.81 | 97 | 343 | 60.2- 60.4 | 85% | 98% | ND |
| benzofuran-2-carboxylic acid 4-fluoro-3- trifluoromethyl- benzylamide | 50 | 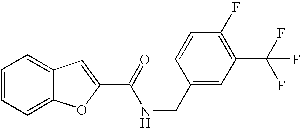 | B | 2.55 | 100 | 338 | 89.5- 91.6 | ND | ND | ND |
| 5-chloro-benzofuran-2- carboxylic acid (1- phenyl-ethyl)-amide | 57 | 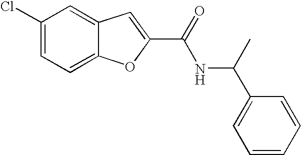 | B | 2.54 | 98 | 300 | 136.3- 138.2 | ND | ND | ND |
| 5-chloro-benzofuran-2- carboxylic acid ((R)-1- phenyl-propyl)-amide | 58 | 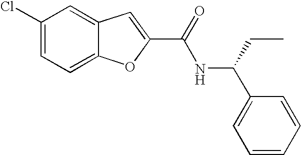 | B | 2.67 | 100 | 314 | 118.5- 120.7 | ND | ND | ND |
| 5-chloro-benzofuran-2- carboxylic acid ((S)-1- phenyl-propyl)-amide | 59 | 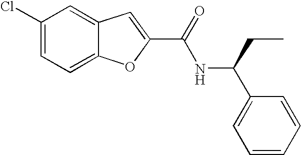 | B | 2.66 | 100 | 314 | 110.1- 111.3 | ND | ND | ND |
| 5-chloro-benzofuran-2- carboxylic acid 2- methylsulfanyl- benzylamide | 60 | 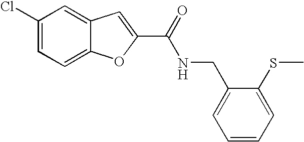 | B | 2.63 | 98 | 332 | 174.7- 177.2 | ND | ND | ND |
| 5-chloro-benzofuran-2- carboxylic acid 4- methylsulfanyl- benzylamide | 61 | 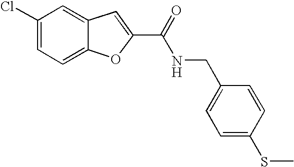 | B | 2.58 | 100 | 332 | 159.0- 161.0 | ND | ND | ND |
| 5-chloro-benzofuran-2- carboxylic acid 2-chloro- 6-methyl-benzylamide | 62 | 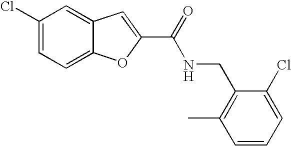 | B | 2.78 | 100 | 335 | 168.3- 168.7 | 39% | 86% | ND |
| 4-{[(5-chloro- benzofuran-2-carbonyl)- amino]-methyl}-benzoic acid methyl ester | 63 | 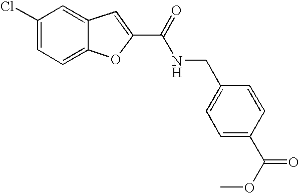 | B | 2.41 | 100 | 344 | — | ND | ND | ND |
| 5-chloro-benzofuran-2- carboxylic acid 4- dimethylamino- benzylamide | 64 | 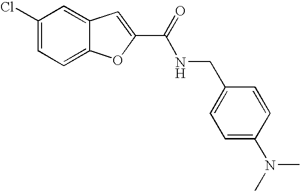 | B | 1.94 | 100 | 329 | 178.6- 179.7 | 76% | 85% | ND |
| 5-{[(5-chloro-benofuran- 2-carbonyl)-amino]- methyl}-benzoic acid methyl ester | 65 | 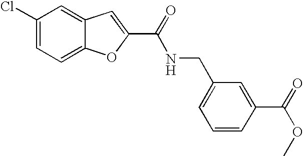 | B | 2.41 | 100 | 344 | 154.0- 155.6 | ND | ND | ND |
| 5-chloro-benzofuran-2- carboxylic acid 3- dimethylcarbamoyl- benzylamide | 66 | 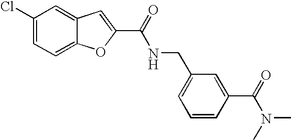 | B | 2.09 | 100 | 357 | 105.1- 109.4 | ND | ND | ND |
| 5-chloro-benzofuran-2- carboxylic acid 4- dimethylsulfamoyl- benzylamide | 67 | 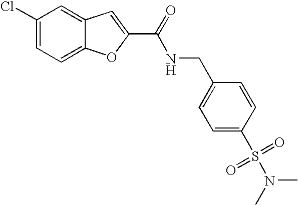 | B | 2.40 | 100 | 393 | 163.9- 166.6 | ND | ND | ND |
| 5-chloro-benzofuran-2- carboxylic acid 4- phenoxy-benzylamide | 68 | 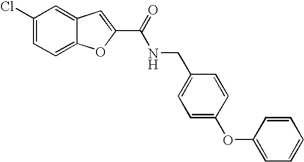 | B | 3.00 | 100 | 378 | 93.6- 95.5 | ND | ND | ND |
| 5-chloro-benzofuran-2- carboxylic acid 2-methyl- benzylamide | 69 | 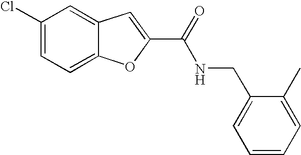 | B | 2.59 | 100 | 300 | 141.8- 143.0 | ND | ND | ND |
| 5-chloro-benzofuran-2- carboxylic acid 3-methyl- benzylamide | 70 | 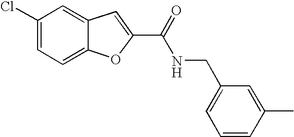 | B | 2.60 | 100 | 300 | 148.4- 149.4 | ND | ND | ND |
| 5-chloro-benzofuran-2- carboxylic acid 4-tert- butyl-benzylamide | 71 | 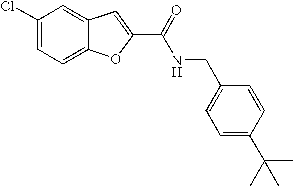 | B | 2.92 | 100 | 342 | 129.5- 131.1 | 71% | 95% | ND |
| 5-chloro-benzofuran-2- carboxylic acid (biphenyl-2-ylmethyl)- amide | 72 | 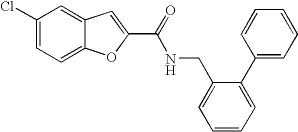 | B | 3.03 | 100 | 362 | 129.8- 132.8 | ND | ND | ND |
| 5-chloro-benzofuran-2- carboxylic acid (biphenyl-3-ylmethyl)- amide | 73 | 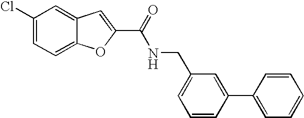 | B | 2.85 | 100 | 362 | 130.4- 131.0 | ND | ND | ND |
| 5-chloro-benzofuran-2- carboxylic acid 3-acetyl- benzylamide | 74 | 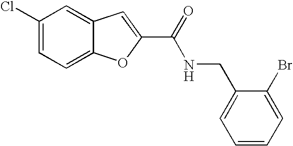 | B | 2.28 | 100 | 328 | 142.9- 147.6 | ND | ND | ND |
| 5-chloro-benzofuran-2- carboxylic acid 2-bromo- benzylamide | 75 | 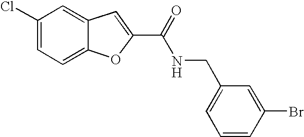 | B | 2.67 | 100 | 365 | 137.2- 139.0 | ND | ND | ND |
| 5-chloro-benzofuran-2- carboxylic acid 3-bromo- benzylamide | 76 | 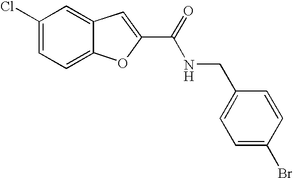 | B | 2.66 | 100 | 365 | 129.9- 131.6 | ND | ND | ND |
| 5-chloro-benzofuran-2- carboxylic acid 4-bromo- benzylamide | 77 | 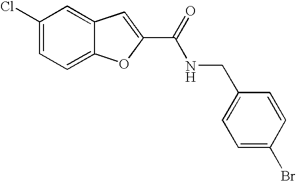 | B | 2.66 | 100 | 365 | 192.3- 193.8 | ND | ND | ND |
| 5-chloro-benzofuran-2- carboxylic acid 4- methanesulfonyl- benzylamide | 79 | 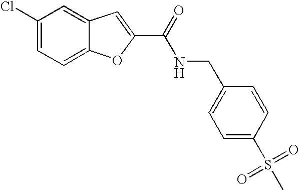 | B | 2.08 | 100 | 364 | 187.6- 189.1 | ND | ND | ND |
| 5-chloro-benzofuran-2- carboxylic acid 4-cyano- benzylamide | 80 | 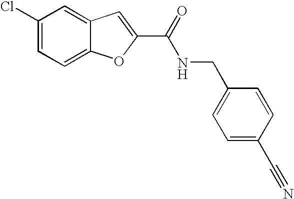 | B | 2.27 | 100 | 311 | 226.6- 230.4 | ND | ND | ND |
| 5-chloro-benzofuran-2- carboxylic acid (pyridin- 4-ylmethyl)-amide | 81 | 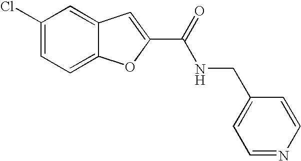 | B | 1.50 | 95 | 287 | 163.1- 166.2 | ND | ND | ND |
| 5-chloro-benzofuran-2- carboxylic acid ((S)-2- hydroxy-1-phenyl-ethyl)- amide | 82 | 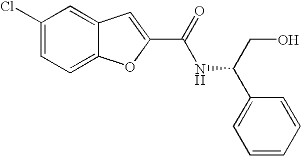 | B | 2.16 | 100 | 316 | ND | ND | ND | ND |
| 5-chloro-benzofuran-2- carboxylic acid ((S)-2- methoxy-1-phenyl-ethyl)- amide | 83 | 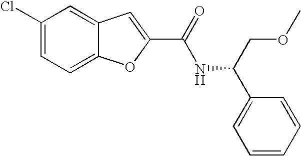 | B | 2.52 | 100 | 330 | — | ND | ND | ND |
| (R)-[(5-chloro- benzofuran-2-carbonyl)- amino]-phenyl-acetic acid methyl ester | 84 | 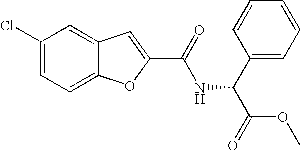 | B | 2.55 | 100 | 344 | 147.9 | ND | ND | ND |
| (S)[(5-chloro- benzofuran-2-carbonyl)- amino]-phenyl-acetic acid methyl ester | 85 | 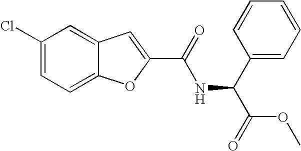 | B | 2.54 | 100 | 344 | 76.5- 79.1 | ND | ND | ND |
| 5-chloro-benzofuran-2- carboxylic acid 2- trifluoromethoxy- benzylamide | 87 | 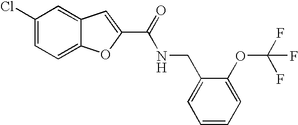 | B | 2.76 | 100 | 370 | 125.8- 127.9 | ND | ND | ND |
| 5-chloro-benzofuran-2- carboxylic acid 3- trifluoromethoxy- benzylamide | 88 | 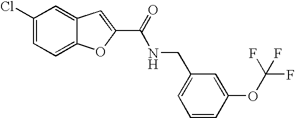 | B | 2.76 | 98 | 370 | 133.5- 137.9 | ND | ND | ND |
| 5-chloro-benzofuran-2- carboxylic acid 4- trifluoromethoxy- benzylamide | 89 | 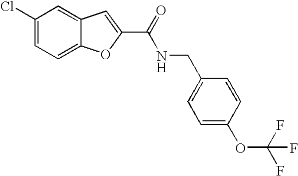 | B | 2.78 | 97 | 370 | 101.3- 106.3 | 37% | 77% | 39% |
| 5-chloro-benzofuran-2- carboxylic acid 2,5- dimethyl-benzylamide | 90 | 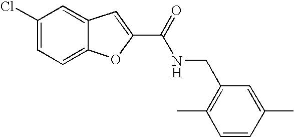 | A | 2.98 | 100 | 328 | 123.1- 124.0 | ND | ND | ND |
| 5-chloro-benzofuran-2- carboxylic acid 4- [1,2,3]thiadiazol-5-yl- benzylamide | 91 | 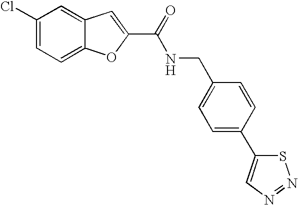 | B | 2.42 | 100 | 370 | 178.1- 181.8 | ND | ND | ND |
| 5-chloro-benzofuran-2- carboxylic acid (benzo[1,3]dioxol-5- ylmethyl)-benzylamide | 92 | 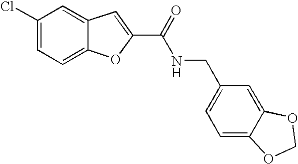 | B | 2.38 | 100 | 330 | 144.0- 145.9 | 64% | 91% | 88% at 1 μM |
| (5-chloro-benzofuran-2- yl)-[4-(4-fluoro-phenyl)- 3,6-dihydro-2H-pyridin- 1-yl]-methanone | 93 | 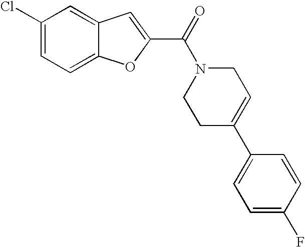 | B | 2.86 | 100 | 356 | 111.3- 116.6 | 73% | 89% | ND |
| 5-chloro-benzofuran-2- carboxylic acid indan-1- ylamide | 94 | 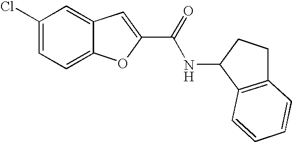 | B | 2.64 | 100 | 312 | 161.4- 164.1 | ND | ND | ND |
| (5-chloro-benzofuran-2- yl)-[4-(2-methoxy- phenyl)-piperidin-1-yl]- methanone | 95 | 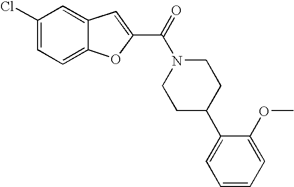 | B | 2.88 | 100 | 370 | 146.1- 147.4 | ND | ND | ND |
| 1-benzyl-1H-indole-3- carboxylic acid 2,4- dimethoxy-benzylamide | 96 | 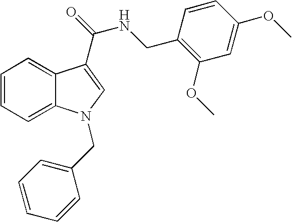 | A | 2.60 | 100 | 401 | 118.8- 120.4 | ND | ND | ND |
| (5-chloro-benzofuran-2- yl)-(4-p-tolyl-piperidin-1- yl)-methanone | 98 | 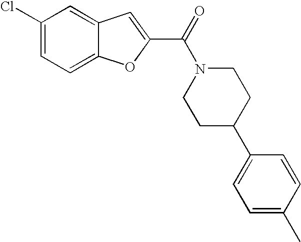 | B | 2.99 | 95 | 354 | — | 80% | 97% | ND |
| (5-chloro-benzofuran-2- carboxylic acid (4- morpholin-4-yl-phenyl)- amide | 99 | 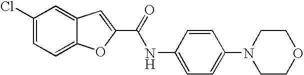 | B | 2.33 | 100 | 357 | 223.8- 225.6 | ND | ND | ND |
| (4-benzyl-piperidin-1-yl)- (5-chloro-benzofuran-2- yl)-methanone | 101 | 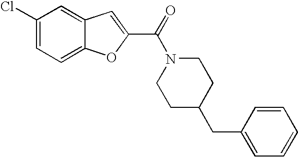 | B | 3.01 | 100 | 354 | 114.6- 116.2 | ND | ND | ND |
| (5-chloro-benzofuran-2- yl)-[4-fluoro-benzoyl)- piperidin-1-yl]- methanone | 102 | 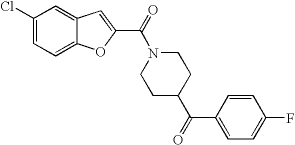 | B | 2.66 | 100 | 386 | 135.0- 137.1 | ND | ND | ND |
| (5-chloro-benzofuran-2- yl)-(2-phenyl-pyrrolidin- 1-yl)-methanone | 103 | 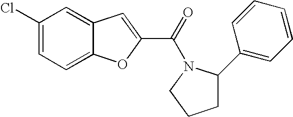 | B | 2.69 | 100 | 326 | 123.3- 125.4 | ND | ND | ND |
| (5-chloro-benzofuran-2- yl)-[2-(4-fluoro-phenyl)- pyrrolidin-1-yl]- methanone | 104 | 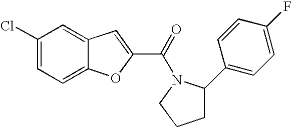 | B | 2.68 | 100 | 344 | 104.4- 106.1 | ND | ND | ND |
| 5-chloro-benzofuran-2- carboxylic acid (4- pyrazol-1-ylmethyl- phenyl)-amide | 105 | 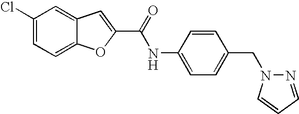 | B | 2.37 | 100 | 352 | 197.4- 198.8 | 90% | 99% | ND |
| (2-benzyl-piperidin-1-yl)- (5-chloro-3-methyl- benzofuran-2-yl)- methanone | 106 | 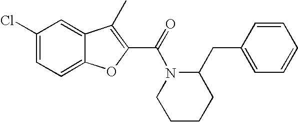 | A | 3.07 | 100 | 368 | — | 59% | 87% | ND |
| 5-chloro-3-methyl- benzo[b]thiophene-2- carboxylic acid 4-fluoro- 3-trifluoromethyl- benzylamide | 107 | 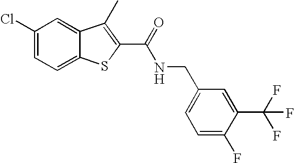 | B | 2.95 | 95 | 402 | 134.0- 136.5 | 30% | 87% | 38% |
| 5-chloro-benzofuran-2- carboxylic acid 2- hydroxy-4-methoxy- benzylamide | 108 | 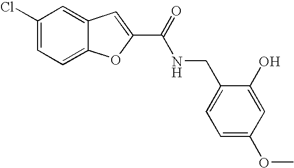 | B | 2.17 | 100 | 332 | 173.8- 176.2 | 85% | 99% | ND |
| 5-chloro-benzofuran-2- carboxylic acid benzyl- (2-cyano-ethyl)-amide | 109 | 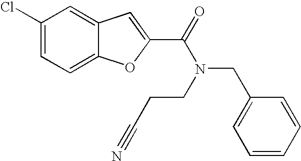 | A | 2.56 | 100 | 339 | — | 74% | 97% | 34% |
| 5-chloro-3-methyl- benzo[b]thiophene-2- carboxylic acid 2-chloro- 6-methyl-benzylamide | 110 | 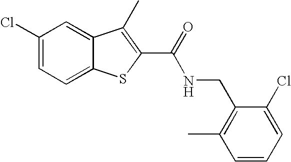 | B | 3.01 | 100 | 365 | 180.5- 182.0 | ND | ND | ND |
| 5-chloro-1H-indole-2- carboxylic acid 2,4- dimethoxy-benzylamide | 111 | 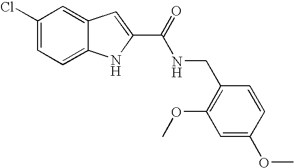 | A | 2.44 | 100 | 345 | 205.7- 209.3 | ND | ND | ND |
| quinolin-2-carboxylic acid 2,4-dimethoxy- benzylamide | 112 | 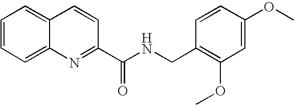 | B | 2.47 | 95 | 323 | 93.9- 95.4 | ND | ND | ND |
| 5-chloro-benzofuran-2- carboxylic acid (2,3- dihydro- benzo[1,4]dioxin-2- ylmethyl)-amide | 113 | 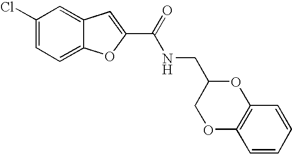 | B | 2.55 | 100 | 344 | 121.1- 124.3 | ND | ND | ND |
| (5-chloro-benzofuran-2- yl)-[4-(2,5-dimethoxy- benzyl)-piperazin-1-yl]- methanone | 114 | 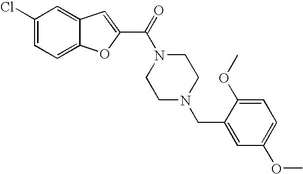 | B | 1.86 | 95 | 415 | — | ND | ND | ND |
| 3-acetylamino-5-chloro- benzofuran-2-carboxylic acid 2,4-dimethoxy- benzylamide | 115 | 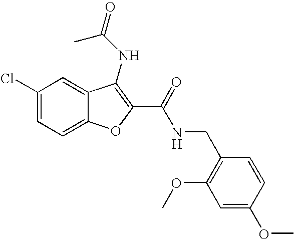 | A | 2.49 | 100 | 403 | 165.5- 170.3 | ND | ND | ND |
| 3-amino-5-chloro- benzofuran-2-carboxylic acid 2,4-dimethoxy- benzylamide | 116 | 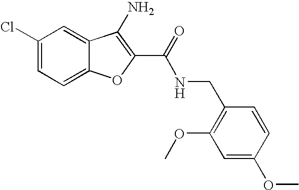 | A | 2.48 | 100 | 361 | — | ND | ND | ND |
| 5-chloro-3-methyl- benzo[b]thiophene-2- carboxylic acid 4- fluoromethoxy- benzylamide | 117 | 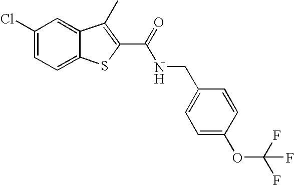 | B | 3.01 | 100 | 400 | 159.2- 162.4 | ND | ND | ND |
| 5-chloro-benzofuran-2- carboxylic acid benzyl- phenyl-amide | 118 | 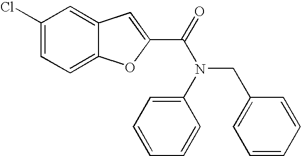 | B | 2.98 | 95 | 362 | — | ND | ND | ND |
| 5-chloro-benzofuran-2- carboxylic acid 2,4- dichloro-6-methyl- benzylamide | 119 | 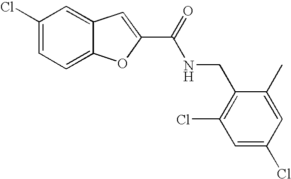 | B | 3.01 | 95 | 369 | 210.0- 212.0 | ND | ND | ND |
| N-(5-chloro- benzooxazol-2-yl)-2- phenyl-acetamide | | | | | | | | | | |

1. A compound having the structural Formula I, II, III or IV, stereoisomers, tautomers, racemics, prodrugs, metabolites thereof, or a pharmaceutically acceptable salt and/or solvate thereof, 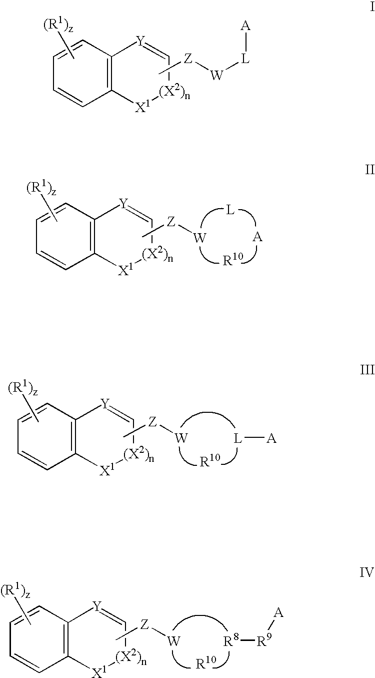
wherein X¹ is a heteroatom selected from —O—, —S—, —N═, or —N(R³)—, wherein R³ is selected from alkyl, aralkyl or alkylcarbonyl,
wherein X² is selected from ═C—, ═CH— or —CH₂—,
wherein n is an integer selected from 0 or 1,
wherein Y is selected from C, —C(R⁵)— or N, wherein R⁵ is selected from hydrogen, amino, alkyl, hydroxyl, alkylamino, heteroaryl, alkylcarbonyloxy, alkylamidyl, or alkylaminocarbonylamino,
wherein Z is selected from —C(═O)—, —CH₂—, or —NH—,
wherein W is selected from —C(═O)—, —N(R²)—, —N(R²)—NH—, —C(═O)—NH—, —CH═, —O— or —CH₂— in formula I, and W is selected from N, or CH in formula II, III or IV,
wherein R¹ is selected from hydrogen, halogen, hydroxy, nitro, amino, azido, cyano, or alkyl, cycloalkyl, alkylamino, alkoxy, carboxy, alkylaminocarbonyl, alkylcarbonyl, heterocyclyl-alkyl, heteroarylalkyl, alkoxycarbonyl, aminocarbonyl, alkylamino(alkylsubstituted)alkyl, alkylcarbonylaminoalkyl or alkylthio, each optionally substituted by one or more substitutents, wherein z is an integer selected from 1, 2, 3 or 4,
wherein R² is selected from hydrogen, alkyl, cycloalkyl, cyanoalkyl, alkenyl, aryl, aminocarbonyl, haloalkyl, aralkyl, cycloalkylalkyl, acyl or alkynyl,
wherein A is selected from aryl, cycloalkyl, heterocyclyl and heteroaryl, each optionally substituted by one or more substituents selected from halogen, hydroxy, nitro, azido, hydrazino, cyano, alkyl, aryl, 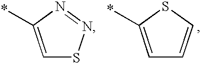
heteroarylalkyl, cycloalkyl, acyl, alkylamino, alkylaminocarbonyl, —SO₂R¹⁵, alkylcarbonyloxy, fused heterocyclyl, haloalkyl, alkylcarbonyl, aryloxy, arylcarbonyl, haloalkoxy, alkoxy, alkylthio, carboxy, acylamino, alkyl esters, carbamate, thioamide, alkyloxycarbonyl, urea, or sulphonamide, wherein R¹⁵ is alkyl, alkylamino or cycloalkyl,
wherein L is a linking group selected from a single bond, a group of formula —R⁸—R⁹—, alkylyn, alkenylyl, cycloalkylene, —NH—(C(R⁴)(R⁴))_{q}—, —(C(R⁴)(R⁴))_{q}—, —(C(R⁴)(R⁴))ᵥ—O—(C(R⁴)(R⁴))_{w}—, —(C(R⁴)(R⁴))ᵥ—N—(C(R⁴)(R⁴))_{w}—, —(C(R⁴)(R⁴))ᵥ—(C(R⁴))_{w}═, —(C(R⁴)(R⁴))_{q}—(C═O)—, or cycloalkylenoxyalkylene, wherein —(C(R⁴)(R⁴))_{q}—, (C(R⁴)(R⁴)), and —(C(R⁴)(R⁴))ᵥ— are each independently aliphatic or form a cycloalkyl, wherein each R⁴ is independently selected from hydrogen, hydroxyl, alkyl, carboxy, hydroxyalkyl, alkoxyalkyl, alkylamino, alkylaminoalkyl or alkyloxycarbonyl; q is an integer selected from 0, 1, 2, 3, 4, 5 or 6; v is an integer selected from 0, 1, 2, 3, 4, 5 or 6 and w is an integer selected from 0, 1, 2, 3, 4, 5 or 6,
wherein R⁸ is alkylyn, —(C(R⁴)(R⁴))ₚ—C(R¹⁴) or —(C(R⁴)(R⁴))ₚ—C(R⁴)═C, wherein R⁹ is selected from a single bond, —(C(R⁴)(R⁴))_{q}—, or —C(═O)—, wherein R¹⁴ is selected from hydrogen, hydroxyl or alkyl, wherein p is an integer selected from 0, 1, 2 or 3 and
wherein R¹⁰ is selected from —(C(R⁴)(R⁴))ₘ—, —(C(R⁴)(R⁴))ₘ—C(═O)O—(C(R⁴)(R⁴))_{q}—, or —(C(R⁴)(R⁴))ₘ—N(R¹²)—(C(R⁴)(R⁴))_{q}—, wherein m is an integer selected from 1, 2, 3, 4, 5 or 6, wherein R¹² is selected from hydrogen, alkyl, aryl, arylalkyl, or alkylcarbonyl.

2. A compound according to claim 1, wherein X¹ is —N(R³)—, n is 0 and wherein R³ has the same meaning as that defined in claim 1.

3. A compound according to claim 1, wherein X¹ is O and n is 0.

4. A compound according to claim 1, wherein X¹ is S and n is 0.

5. A compound according to claim 1, wherein X¹ is —N═ and X² is ═CH— or C and n is 1.

6. A compound according to claim 1, having one of the structural Formula V, VI, VII, VIII, IX or X 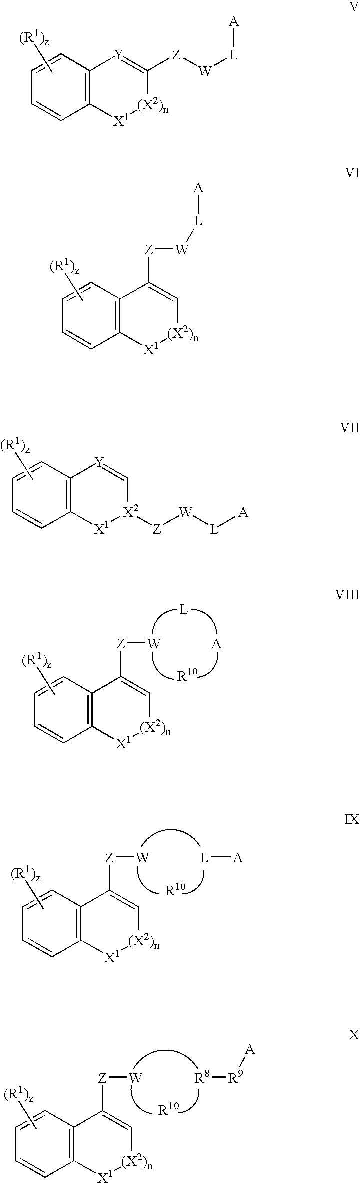
wherein R¹, z, X¹, X², W, Z, n, L, A, R⁸, R⁹, R¹⁰ and R² have the same meaning as that defined in any of the previous claims.

7. A compound according to claim 1, having one of the structural Formula X, XI, XII, XIII, XIV or XV 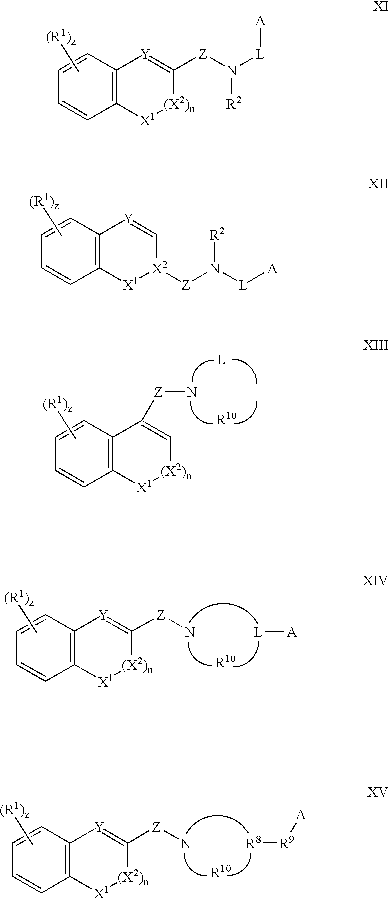
wherein R¹, z, X¹, X², Y, Z, n, L, A, R⁸, R⁹, R¹⁰ and R² have the same meaning as that defined in any of the previous claims.

8. A compound according to claim 1, having one of the structural Formula XVI to XXXI 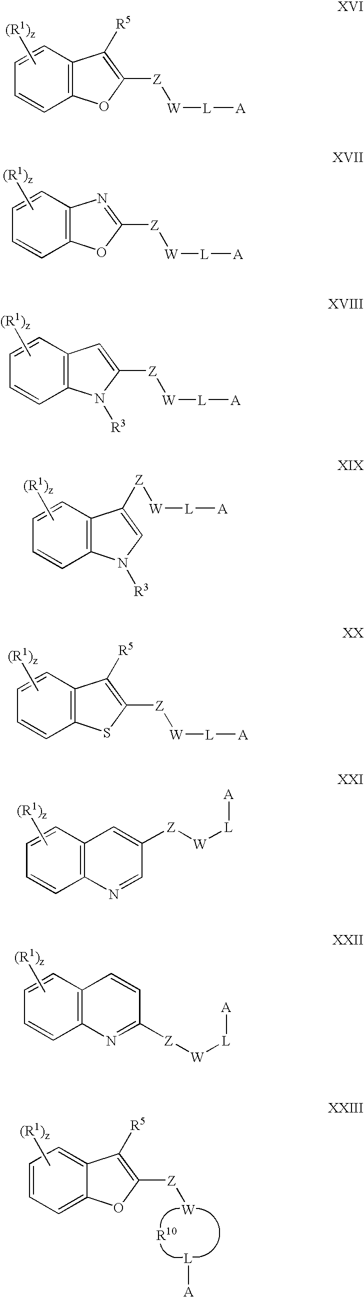 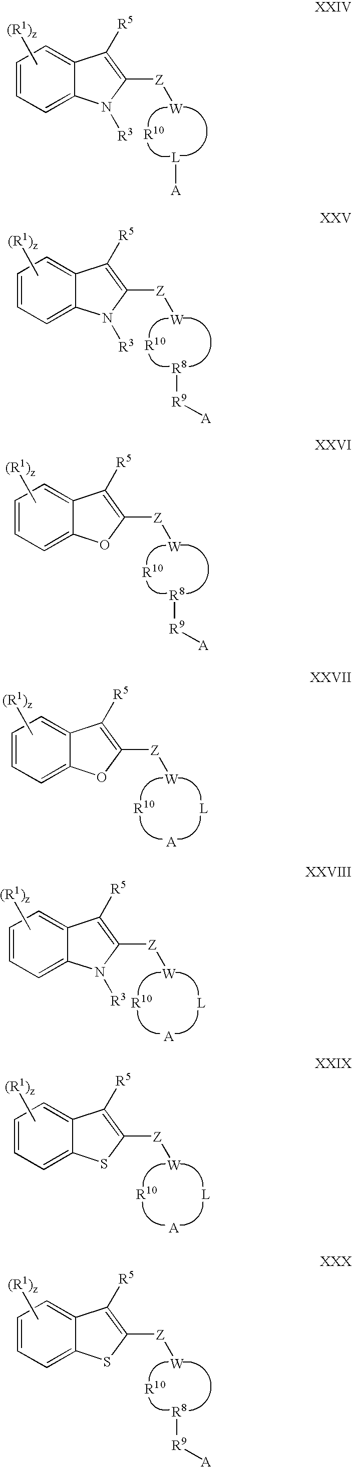 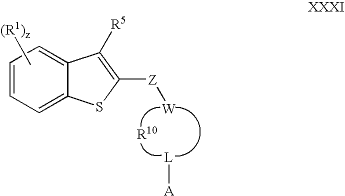
wherein R⁵ is selected from hydrogen, alkyl, or aralkyl and wherein R¹, z, Z, W, L, A, R⁸, R⁹, R¹⁰ and R³ have the same meaning as that defined in any of the previous claims.

9. A compound according to claim 1, having one of the structural Formula XXXII to LVII 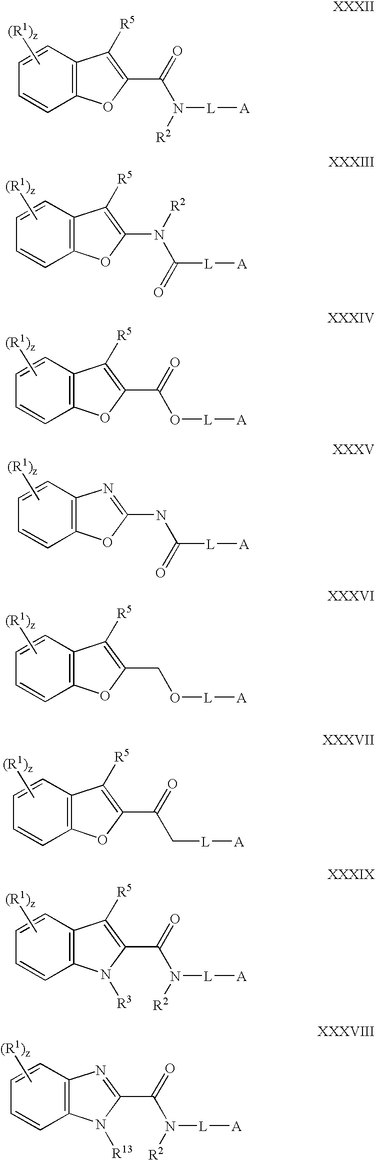 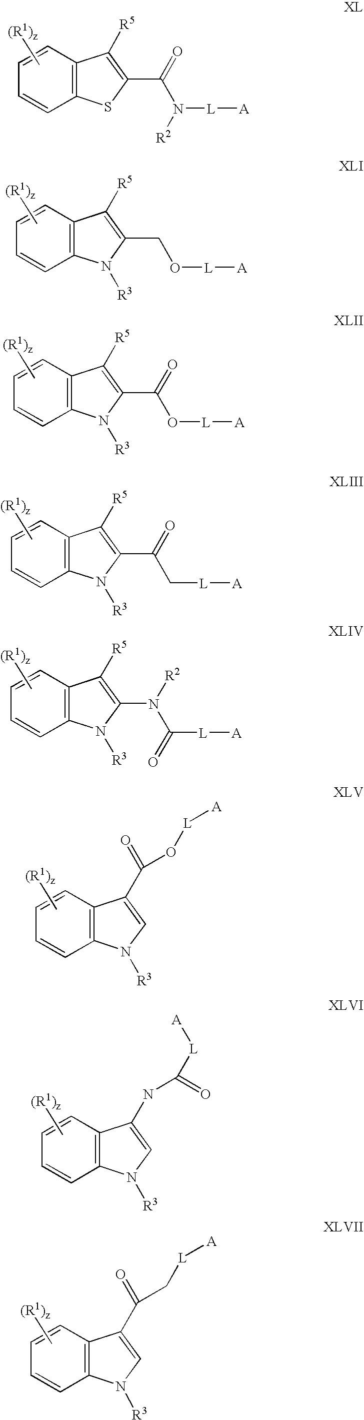 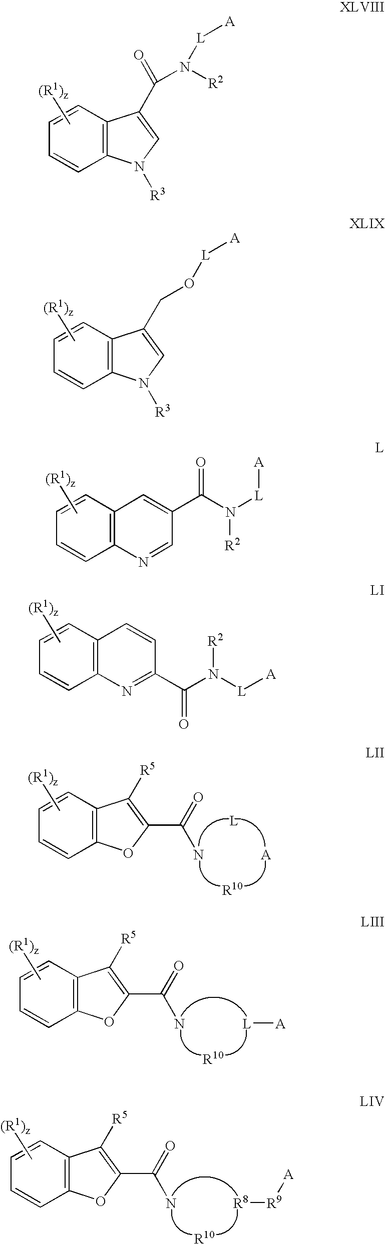 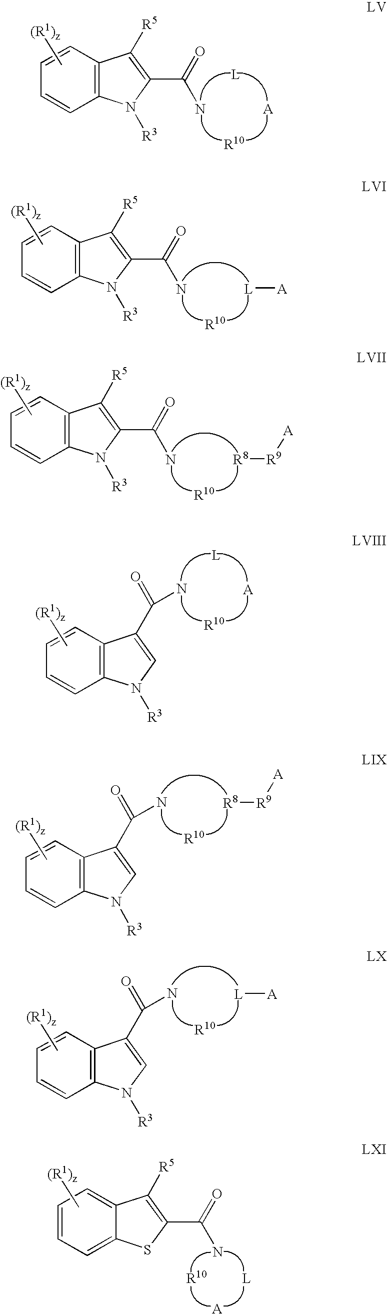 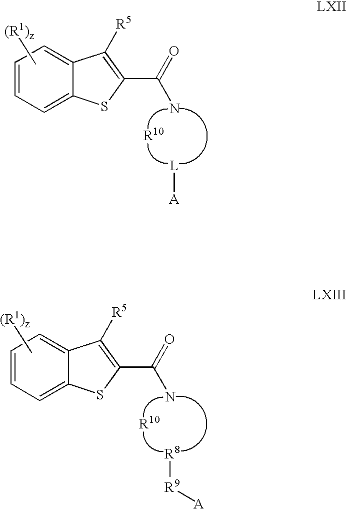
wherein R¹, z, R⁵, R², R³, R⁸, R⁹, R¹⁰, L and A have the same meaning as that defined in any of the previous claims.

10. A compound according to claim 1, wherein A is selected from 2- or 3-furyl, 2- or 3-thienyl, 1-, 2- or 3-pyrrolyl, 1-, 2-, 4- or 5-imidazolyl, 1-, 3-, 4- or 5-pyrazolyl, 3-, 4- or 5-isoxazolyl, 2-, 4- or 5-oxazolyl, 3-, 4- or 5-isothiazolyl, 2, 4- or 5-thiazolyl, 1,2,3-triazol-1-, -2-, -4- or -5-yl, 1,2,4-triazol-1-, -3-, -4- or -5-yl, 1,2,3-oxadiazol-4- or -5-yl, 1,2,4-oxadiazol-3- or -5-yl, 1,2,3-thiadiazol-4- or -5-yl, 1,2,4-thiadiazol-3- or -5-yl, 1,2,5-thiadiazol-3- or -4-yl, 1- or 5-tetrazolyl, phenyl, biphenyl, 2-, 3- or 4-pyridyl, pyridinyl, anthracenyl, azulenyl, indenyl, 3- or 4-pyridazinyl, 2-, 4-, 5- or 6-pyrimidinyl, 2-, 3-, 4-, 5- 6-2H-thiopyranyl, 2-, 3- or 4-4H-thiopyranyl, furyl, 2-, 3-, 4-, 5-, 6- or 7-benzofuryl, 2-, 3-, 4-, 5-, 6- or 7-benzothienyl, 1-, 2-, 3-, 4-, 5-, 6- or 7-indolyl, 1-, 2-, 4- or 5-benzimidazolyl, 1-, 3-, 4-, 5-, 6- or 7-benzopyrazolyl, 3-, 4-, 5-, 6- or 7-benzisoxazolyl, 2-, 4-, 5-, 6- or 7-benzoxazolyl, 3-, 4-, 5-, 6- or 7-benzisothiazolyl, 2-, 4-, 5-, 6- or 7-benzothiazolyl, 1,3-benzodioxolyl, 1- or 2-naphthyl, 2-, 3-, 4-, 5-, 6-, 7-, 8-quinolinyl, 2-, 4-, 5-, 6-, 7- or 8-quinazolyl, 1-, 3-, 4-, 5-, 6-, 7-, 8-isoquinolinyl, or 1-, 2-, 3-, 4- or 9-carbazolyl, 5,6,7,8-tetrahydronaphthyl, thienyl, benzothienyl, dibenzo[b,f]azepinyl, dibenzo[a,d]cylcoheptenyl, pyrrolyl, dioxanyl, thietanyl, oxazolyl, piperidinyl, imidazolinyl, isoxazolinyl, oxazolidinyl, thiazolidinyl, isothiazolidinyl, 2-oxopiperazinyl, pyrazinyl, triazinyl, cinnolinyl, phthalazinyl, oxetanyl, azepinyl, 4-piperidonyl, 2-oxopiperidinyl, 2-oxopyrrolodinyl, 5-(2,3-dihydro)benzofuranyl, 2-oxoazepinyl, tetrahydropyranyl, thiamorpholinyl, thiamorpholinyl sulfoxide, thiamorpholinyl sulfone, 1,3-dioxolane, tetrahydro-1,1-dioxothienyl, piperazinyl or morpholinyl, each optionally substituted by 1, 2, 3 or 4 substituents selected from halogen, hydroxy, nitro, amino, azido, hydrazino, cyano, alkyl, aryl, heteroarylalkyl, cycloalkyl, alkyloxycarbonyl, acyl, alkylamino, alkylaminocarbonyl, alkylcarbonyloxy, fused heterocyclyl, haloalkyl, —SO₂R¹⁵, alkylcarbonyl, aryloxy, arylcarbonyl, haloalkoxy, alkoxy, thiol, alkylthio, carboxy, acylamino, alkyl esters, carbamate, thioamide, urea, or sulphonamide, wherein R¹⁵ is alkyl, alkylamino or cycloalkyl,
wherein L is selected from a single bond, a group of formula —R⁸—R⁹—, alkylyn, alkenylyl, cycloalkylene, —NH—(C(R⁴)(R⁴))_{q}—, —(C(R⁴)(R⁴))_{q}—, —(C(R⁴)(R⁴))ᵥ—O—(C(R⁴)(R⁴))_{w}—, —(C(R⁴)(R⁴))ᵥ—N—(C(R⁴)(R⁴))_{w}—, —(C(R⁴)(R⁴))ᵥ—(C(R⁴))_{w}═, (C(R⁴)(R⁴))_{q}—(C═O)—, or cycloalkylenoxyalkylene, wherein —(C(R⁴)(R⁴))_{q}—, (C(R⁴)(R⁴))_{w} and —(C(R⁴)(R⁴))ᵥ— are each independently aliphatic or form a cycloalkyl, wherein each R⁴ is independently selected from hydrogen, alkyl, hydroxyl, carboxy, hydroxyalkyl, alkoxyalkyl, alkylamino, alkylaminoalkyl or alkyloxycarbonyl; q is an integer selected from 0, 1, 2, 3 or 4; v is an integer selected from 0, 1, 2, 3 or 4 and w is an integer selected from 0, 1, 2, 3, or 4,
wherein R⁸ is alkylyn, —(C(R⁴)(R⁴))ₚ—C(R¹⁴) or —(C(R⁴)(R⁴))ₚ—C(R⁴)═C, wherein R⁹ is a single bond, —(C(R⁴)(R⁴))_{q}—, or —C(═O)—, wherein R¹⁴ is selected from hydrogen, hydroxyl or alkyl, wherein p is an integer selected from 0, 1, 2 or 3, and
wherein R¹⁰ is selected from —(C(R⁴)(R⁴))ₘ—, —(C(R⁴)(R⁴))ₘ—C(═O)O—(C(R⁴)(R⁴))_{q}—, or —(C(R⁴)(R⁴))ₘ—N(R¹²)—(C(R⁴)(R⁴))_{q}—, wherein m is an integer selected from 1, 2, 3, or 4,
wherein R¹² is selected from hydrogen, alkyl, aryl, arylalkyl, or alkylcarbonyl and
wherein R² is selected from hydrogen, CH₃—, —CH₂—CH₃, —(CH₂)₂—CH₃, —(CH₂)₂—CN, phenyl, benzyl or CH₃—C(═O).

11. A compound according to claim 1, having a structural formula selected from Formula XVI to XXII and XXXII to LI, wherein A is selected from phenyl, 3-indolyl, 5-(2,3-dihydro)benzofuranyl, 6-indolyl, 4-pyridinyl, 1,3-benzodioxolyl, 2-thienyl, 2-naphthyl, dibenzo[b,f]azepinyl, dibenzo[a,d]cylcoheptenyl, each optionally substituted by 1; 2, 3 or 4 substituents selected from —OCH₃, —NO₂, —CO₂H, —C(═O)—N(CH₃)₂, —O—C(═O)—CH₃, —CH₃, —CH₂—CH₃, phenyl, —SO₂—CH₃, F, Cl, Br, —CF₃, —S—CH₃, —OCF₃, —C(═O)—CH₃, —O—C(═O)—CH₃, —C(═O)O—CH₃, —C(═O)N(CH₃)₂, —N(CH₃)₂, —SO₂—N(CH₃)₂, 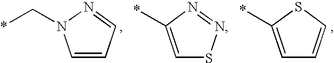
N-morpholino, phenoxyl, benzoyl, —C(CH₃)₃, —O—(CH₂)₂—CH₃, —OH or —CN,
wherein L is selected from single bond, —CH₂—, —(CH₂)₂—, —(CH₂)₃—, —CH(CH₂OH)—, —CH(CH₂—O—CH₃)—, —CH(CH₃)—, —CH(CH₂—CH₃)—, —CH(CO₂H)—, —CH(CO₂CH₃)—, —(CH₂)₂—O—CH₂—, —CH(CH₂—N(CH₃)₂)—, —(CH₂)₂—CH═, or 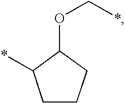
or wherein -L-A is 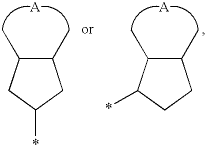
wherein R¹ is selected from hydrogen, Cl, Br, F, —CF₃, —OCH₃, CH₃—C(═O)—, —CO₂H, (CH₃)₂N—C(═O)—, 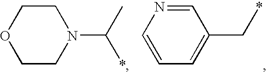
CH₃—C(═O)O—, CH₃—O—C(═O)—, CH₃—NH—C(═O)—, CH₃—C(═O)—NH—CH(CH₃)—, HO—CH₂—, CH₃—CH₂—, CH₃—O—CH₂—, wherein z is 1 or 2,
wherein R⁵ is selected from H, —NH₂, CH₃—NH—C(═O)—NH—, CH₃—C(═O)—NH—, CH₃—C(═O)O—, —OH, —CH₃, CH₃—CH₂—, (CH₃)₂N—, (CH₃)₂N—CH(CH₃)—, or 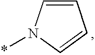
wherein R² is selected from hydrogen, CH₃—, CH₃—, —CH₂—CH₃, —(CH₂)₂—CH₃, —(CH₂)₂—CN, phenyl, benzyl or CH₃—C(═O).

12. A compound according to claim 1, having a structural formula selected from Formula XXIII to XXXI and LII to LXIII,
wherein the group 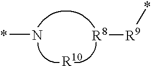
is selected from 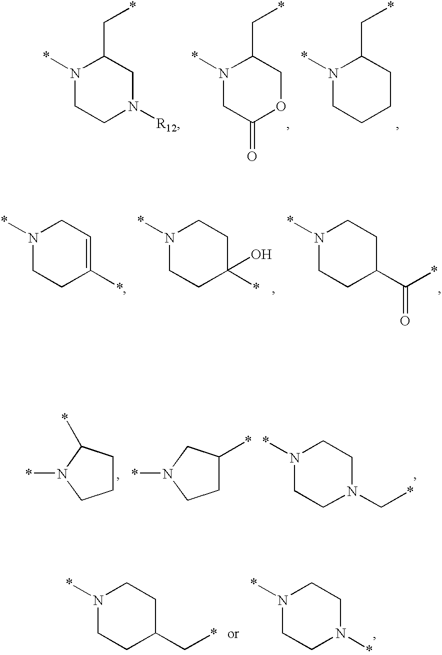
wherein the group 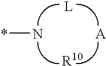
is selected from 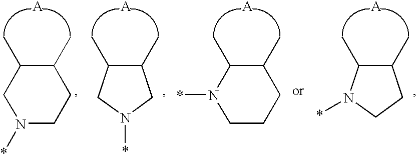
wherein the group 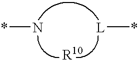
is selected from 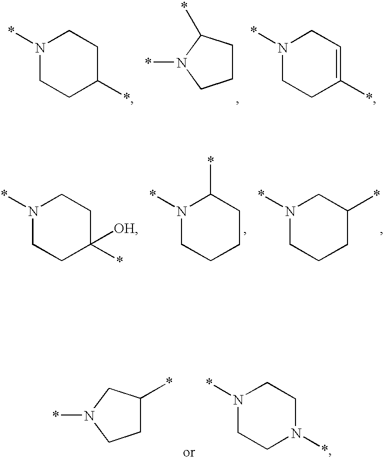
wherein A is selected from phenyl, 3-indolyl, 5-(2,3-dihydro)benzofuranyl, 6-indolyl, 4-pyridinyl, 2-thienyl, 1,3-benzodioxolyl, 2-naphthyl, dibenzo[b,f]azepinyl, dibenzo[a,d]cylcoheptenyl, each optionally substituted by 1, 2, 3 or 4 substituents selected from —OCH₃, —NO₂, —CO₂H, —C(═O)—N(CH₃)₂, —O—C(═O)—CH₃, —CH₃, —CH₂—CH₃, phenyl, —SO₂—CH₃, F, Cl, Br, —CF₃, —S—CH₃, —OCF₃, —C(═O)—CH₃, —O—C(═O)—CH₃, —C(═O)O—CH₃, —C(═O)N(CH₃)₂, —N(CH₃)₂, —SO₂—N(CH₃)₂, 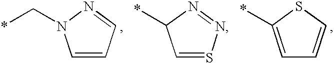
N-morpholino, phenoxyl, benzoyl, —C(CH₃)₃, —O—(CH₂)₂—CH₃, —OH or —CN,
wherein R¹² is selected from hydrogen, CH₃—C(═O)—, CH₃— or benzyl,
wherein R¹ is selected from hydrogen, Cl, Br, F, —CF₃, —OCH₃, CH₃—C(═O)—, —CO₂H, (CH₃)₂N—C(═O)—, 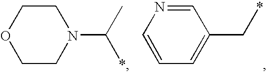
CH₃—C(═O)O—, CH₃—O—C(═O)—, CH₃—NH—C(═O)—, CH₃—C(═O)—NH—CH(CH₃)—, HO—CH₂—, CH₃—CH₂—, CH₃—O—CH₂—, wherein z is 1 or 2,
wherein R⁵ is selected from H, CH₃—NH—C(═O)—NH—, CH₃—C(═O)—NH—, CH₃—C(═O)O—, —OH, —CH₃, CH₃—CH₂—, (CH₃)₂N—, or 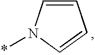
wherein R² is selected from hydrogen, CH₃—, phenyl, CH₃—, —CH₂—CH₃, —(CH₂)₂—CH₃, —(CH₂)₂—CN, benzyl or CH₃—C(═O).

13. A compound according to claim 1, wherein
wherein R⁵ is selected from hydrogen, CH₃—, —NH₂, CH₃—C(═O)—NH—, or 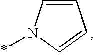
wherein R² is selected from hydrogen, CH₃—, phenyl, CH₃—, —CH₂—CH₃, —(CH₂)₂—CH₃, or —(CH₂)₂—CN,
wherein R¹ is selected from H, Cl, F, Br, —OCH₃, —C(═O)CH₃, wherein z is an integer selected from 1, 2 or 3,
wherein A is selected from phenyl, 3-indolyl, 5-(2,3-dihydro)benzofuranyl, 6-indolyl, 4-pyridinyl, 2-thienyl, 1,3-benzodioxolyl, 2-naphthyl, dibenzo[b,f]azepinyl, dibenzo[a,d]cylcoheptenyl, each optionally substituted by 1; 2 or 3 substituents selected from —OCH₃, —NO₂, —CO₂H, —C(═O)—N(CH₃)₂, —O—C(═O)—CH₃, —CH₃, —CH₂—CH₃, phenyl, —SO₂—CH₃, F, Cl, Br, —CF₃, —S—CH₃, —OCF₃, —C(═O)—CH₃, —O—C(═O)—CH₃, —C(═O)O—CH₃, —C(═O)N(CH₃)₂, —N(CH₃)₂, —SO₂—N(CH₃)₂, 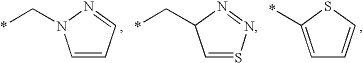
N-morpholino, phenoxyl, benzoyl, —C(CH₃)₃, —O—(CH₂)₂—CH₃, —OH or —CN,
wherein L is a linking group selected from a single bond, a group of formula —R⁸—R⁹—, alkylyn, alkenylyl, cycloalkylene, —NH—(C(R⁴)(R⁴))_{q}—, —(C(R⁴)(R⁴))_{q}—, —(C(R⁴)(R⁴))ᵥ—O—(C(R⁴)(R⁴))_{w}—, —(C(R⁴)(R⁴))ᵥ—N—(C(R⁴)(R⁴))_{w}—, —(C(R⁴)(R⁴))ᵥ—(C(R⁴))_{w}═, —(C(R⁴)(R⁴))_{q}—(C═O)—, or cycloalkylenoxyalkylene, wherein —(C(R⁴)(R⁴))_{q}—, (C(R⁴)(R⁴))_{w} and —(C(R⁴)(R⁴))ᵥ— are each independently aliphatic or form a cycloalkyl, wherein each R⁴ is independently selected from hydrogen, hydroxyl, alkyl, carboxy, hydroxyalkyl, alkoxyalkyl, alkylamino, alkylaminoalkyl or alkyloxycarbonyl; q is an integer selected from 0, 1, 2, 3, 4, 5 or 6; v is an integer selected from 0, 1, 2, 3, 4, 5 or 6 and w is an integer selected from 0, 1, 2, 3, 4, 5 or 6,
wherein R⁸ is alkylyn, —(C(R⁴)(R⁴))ₚ—C(R¹⁴) or —(C(R⁴)(R⁴))ₚ—C(R⁴)═C, wherein R⁹ is selected from a single bond, —(C(R⁴)(R⁴))_{q}—, or —C(═O)—, wherein R¹⁴ is selected from hydrogen, hydroxyl or alkyl, wherein p is an integer selected from 0, 1, 2 or 3 and
wherein R¹⁰ is selected from —(C(R⁴)(R⁴))ₘ—, —(C(R⁴)(R⁴))ₘ—C(═O)O—(C(R⁴)(R⁴))_{q}—, or —(C(R⁴)(R⁴))ₘ—N(R¹²)—(C(R⁴)(R⁴))_{q}—, wherein m is an integer selected from 1, 2, 3, 4, 5 or 6, wherein R¹² is selected from hydrogen, alkyl, aryl, arylalkyl, or alkylcarbonyl.

14. A compound according to claim 1 selected from the group comprising 5-chloro-benzofuran-2-carboxylic acid [(R)-1-(4-nitrophenyl)-ethyl]-amide; 5-chloro-benzofuran-2-carboxylic acid 2,4-dimethoxy-benzylamide; 5-chloro-benzofuran-2-carboxylic acid [2-(5-methyl-1H-indol-3-yl)-ethyl]-amide; 5-chlorobenzofuran-2-carboxylic acid [3-(10,11-dihydro-dibenzo[b,f]azepin-5-yl)-propyl]-methylamide; 5-chloro-benzofuran-2-carboxylic acid [3-(10,11-dihydro-dibenzo[a,d]cyclohepten-5-ylidene)-propyl]-methyl-amide; 5-chloro-benzofuran-2-carboxylic acid (4-nitro-benzyl)propyl-amide; (5-chloro-benzofuran-2-yl)-[4-(4-chloro-benzoyl)-piperidin-1-yl]-methanone; 5-chloro-benzofuran-2-carboxylic acid 4-dimethylamino-benzylamide; 7-methoxybenzofuran-2-carboxylic acid [(R)-1-(4-nitro-phenyl)-ethyl]-amide; 7-methoxy-benzofuran-2-carboxylic acid ((S)-1-naphthalen-2-yl-ethyl) amide; 7-methoxy-benzofuran-2-carboxylic acid ((1R,2R)-2-benzyloxy-cyclopent-1-yl)-amide; benzofuran-2-carboxylic acid 2,4-dimethoxy-benzylamide; 4-acetyl-7-methoxy-benzofuran-2-carboxylic acid 2,4-dimethoxy-benzylamide; 5-chloro-3-methyl-benzofuran-2-carboxylic acid 2,4-dimethoxy-benzylamide; 3-pyrrol-1-yl-benzofuran-2-carboxylic acid 2,4-dimethoxy-benzylamide; 5-chloro-1-methyl-1H-indole-2-carboxylic acid 2,4-dimethoxy-benzylamide; 5-chloro-3-methyl-benzo[b]thiophene-2-carboxylic acid 2,4-dimethoxy-benzylamide; N-benzofuran-2-yl-2-(2,4-dimethoxy-phenyl)-acetamide; 1-benzofuran-2-yl-2-(2,4-dimethoxy-phenyl)-ethanone; 5-chloro-benzofuran-2-carboxylic acid (2,4-dimethoxy-phenyl)-amide; 5-chloro-benzofuran-2-carboxylic acid indan-2-ylamide; (5-chloro-benzofuran-2-yl)-(1,3-dihydro-isoindol-2-yl)-methanone; (5-chloro-benzofuran-2-yl)-(3,4-dihydro-1H-isoquinolin-2-yl)-methanone; (2-benzyl-piperidin-1-yl)-(5-chloro-benzofuran-2-yl)-methanone; benzo[b]thiophene-2-carboxylic acid 2,4-dimethoxy-benzylamide; acetic acid 4-{[(5-chloro-benzofuran-2-carbonyl)-amino]-methyl}-phenyl ester; 5-chloro-benzofuran-2-carboxylic acid 4-thiophen-2-yl-benzylamide; 5-chloro-benzofuran-2-carboxylic acid 4-methyl-benzylamide; (5-chloro-benzofuran-2-yl)-(4-phenyl-piperidin-1-yl)-methanone; 5-chloro-benzofuran-2-carboxylic acid 4-(thiophen-2-ylmethyl) amide; (S)-[(5-chloro-benzofuran-2-carbonyl)-amino]-phenyl-acetic acid; quinoline-3-carboxylic acid 2,4-dimethoxy-benzylamide; 3-methyl-benzofuran-2-carboxylic acid 2,4-dimethoxy-benzylamide; 5-chloro-benzofuran-2-carboxylic benzyl ester; 5-chloro-benzofuran-2-carboxylic benzylamide; 5-chloro-benzofuran-2-carboxylic acid (2-dimethylamino-1-phenyl-ethyl)-amide.

15. A compound according to claim 1 selected from the group comprising benzofuran-2-carboxylic acid 4-fluoro-3-trifluoromethyl-benzylamide; 5-chloro-benzofuran-2-carboxylic acid (1-phenyl-ethyl)-amide; 5-chloro-benzofuran-2-carboxylic acid ((R)-1-phenyl-propyl)-amide; 5-chloro-benzofuran-2-carboxylic acid ((S)-1-phenyl-propyl)-amide; 5-chloro-benzofuran-2-carboxylic acid 2-methylsulfanyl-benzylamide; 5-chloro-benzofuran-2-carboxylic acid 4-methylsulfanyl-benzylamide; 5-chloro-benzofuran-2-carboxylic acid 2-chloro-6-methyl-benzylamide; 4-{[(5-chloro-benzofuran-2-carbonyl)-amino]-methyl}-benzoic acid methyl ester; 5-chloro-benzofuran-2-carboxylic acid 4-dimethylamino-benzylamide; 3-{[(5-chloro-benzofuran-2-carbonyl)-amino]-methyl}-benzoic acid methyl ester; 5-chloro-benzofuran-2-carboxylic acid 3-dimethylcarbamoyl-benzylamide; 5-chloro-benzofuran-2-carboxylic acid 4-dimethylsulfamoyl-benzylamide; 5-chloro-benzofuran-2-carboxylic acid 4-phenoxy-benzylamide; 5-chloro-benzofuran-2-carboxylic acid 2-methyl-benzylamide; 5-chloro-benzofuran-2-carboxylic acid 3-methyl-benzylamide; 5-chloro-benzofuran-2-carboxylic acid 4-tert-butyl-benzylamide; 5-chloro-benzofuran-2-carboxylic acid (biphenyl-2-ylmethyl)-amide; 5-chloro-benzofuran-2-carboxylic acid (biphenyl-3-ylmethyl)-amide; 5-chloro-benzofuran-2-carboxylic acid 3-acetyl-benzylamide; 5-chloro-benzofuran-2-carboxylic acid 2-bromo-benzylamide; 5-chloro-benzofuran-2-carboxylic acid 3-bromo-benzylamide; 5-chloro-benzofuran-2-carboxylic acid 4-bromo-benzylamide; 5-chloro-benzofuran-2-carboxylic acid 4-methanesulfonyl-benzylamide; 5-chloro-benzofuran-2-carboxylic acid 4-cyano-benzylamide; 5-chloro-benzofuran-2-carboxylic acid (pyridin-4-ylmethyl)-amide; 5-chloro-benzofuran-2-carboxylic acid ((S)-2-hydroxy-1-phenyl-ethyl)-amide; 5-chloro-benzofuran-2-carboxylic acid ((S)-2-methoxy-1-phenyl-ethyl)-amide; (R)-[(5-chloro-benzofuran-2-carbonyl)-amino]-phenyl-acetic acid methyl ester; (S)-[(5-chloro-benzofuran-2-carbonyl)-amino]-phenyl-acetic acid methyl ester; 5-chloro-benzofuran-2-carboxylic acid 2-trifluoromethoxy-benzylamide; 5-chloro-benzofuran-2-carboxylic acid 3-trifluoromethoxy-benzylamide; 5-chloro-benzofuran-2-carboxylic acid 4-trifluoromethoxy-benzylamide.

16. A compound according to claim 1 selected from the group comprising 5-chloro-benzofuran-2-carboxylic acid 2,5-dimethyl-benzylamide; 5-chloro-benzofuran-2-carboxylic acid 4-[1,2,3]thiadiazol-5-yl-benzylamide; 5-chloro-benzofuran-2-carboxylic acid (benzo[1,3]dioxol-5-ylmethyl)-benzylamide; (5-chloro-benzofuran-2-yl)-[4-(4-fluoro-phenyl)-3,6-dihydro-2H-pyridin-1-yl]-methanone; 5-chloro-benzofuran-2-carboxylic acid indan-1-ylamide; (5-chloro-benzofuran-2-yl)-[4-(2-methoxy-phenyl)-piperidin-1-yl]-methanone; 1-benzyl-1H-indole-3-carboxylic acid 2,4-dimethoxy-benzylamide; (5-chloro-benzofuran-2-yl)-(4-p-tolyl-piperidin-1-yl)-methanone; 5-chloro-benzofuran-2-carboxylic acid (4-morpholin-4-yl-phenyl)-amide; (4-benzyl-piperidin-1-yl)-(5-chloro-benzofuran-2-yl)-methanone; (5-chloro-benzofuran-2-yl)-[4-(4-fluoro-benzoyl)-piperidin-1-yl]-methanone; (5-chloro-benzofuran-2-yl)-(2-phenyl-pyrrolidin-1-yl)-methanone; (5-chloro-benzofuran-2-yl)-[2-(4-fluoro-phenyl)-pyrrolidin-1-yl]-methanone; 5-chloro-benzofuran-2-carboxylic acid (4-pyrazol-1-ylmethyl-phenyl)-amide; (2-benzyl-piperidin-1-yl)-(5-chloro-3-methyl-benzofuran-2-yl)-methanone; 5-chloro-3-methyl-benzo[b]thiophene-2-carboxylic acid 4-fluoro-3-trifluoromethyl-benzylamide; 5-chloro-benzofuran-2-carboxylic acid-2-hydroxy-4-methoxy-benzylamide; 5-chloro-benzofuran-2-carboxylic acid benzyl-(2-cyano-ethyl)-amide; 5-chloro-3-methyl-benzo[b]thiophene-2-carboxylic acid 2-chloro-6-methyl-benzylamide; 5-chloro-1H-indole-2-carboxylic acid 2,4-dimethoxy-benzylamide; quinoline-2-carboxylic acid 2,4-dimethoxy-benzylamide; 5-chloro-benzofuran-2-carboxylic acid (2,3-dihydro-benzo[1,4]dioxin-2-ylmethyl)-amide; (5-chloro-benzofuran-2-yl)-[4-(2,5-dimethoxy-benzyl)-piperazin-1-yl]-methanone; 3-acetylamino-5-chloro-benzofuran-2-carboxylic acid 2,4-dimethoxy-benzylamide; 3-amino-5-chloro-benzofuran-2-carboxylic acid 2,4-dimethoxy-benzylamide; 5-chloro-3-methyl-benzo[b]thiophene-2-carboxylic acid 4-fluoromethoxy-benzylamide; 5-chloro-benzofuran-2-carboxylic acid benzyl-phenyl-amide; 5-chloro-benzofuran-2-carboxylic acid 2,4-dichloro-6-methyl-benzylamide; N-(5-chloro-benzooxazol-2-yl)-2-phenyl-acetamide.

17. A compound according to claim 1, wherein said compound is 5-chlorobenzofuran-2-carboxylic acid 3,5-dimethoxybenzyl-amide or 5-chlorobenzofuran-2-carboxylic acid [3-(10,11-dihydro-dibenzo[b,f]azepin-5-yl)propyl]methyl-amide, or 5-chloro-3-methyl-benzo[b]thiophene-2-carboxylic acid 2,4-dimethoxy-benzylamide.

18. A compound according to claim 1, wherein said compound is 5-chlorobenzofuran-2-carboxylic acid 3,5-dimethoxybenzyl-amide or 5-chloro-3-methyl-benzo[b]thiophene-2-carboxylic acid 2,4-dimethoxy-benzylamide.

19. Method for synthesizing a compound having the structural Formula I, II, III or IV comprising the step of condensing a compound of Formula LXIV: 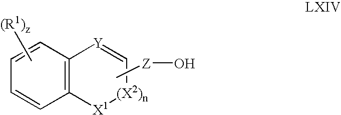
with a compound of Formula LXV, LXVI, LXVII or LXVIII: 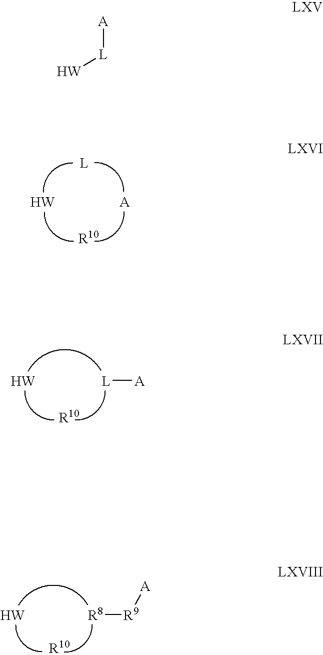
thereby obtaining a compound of Formula I, II, III or IV 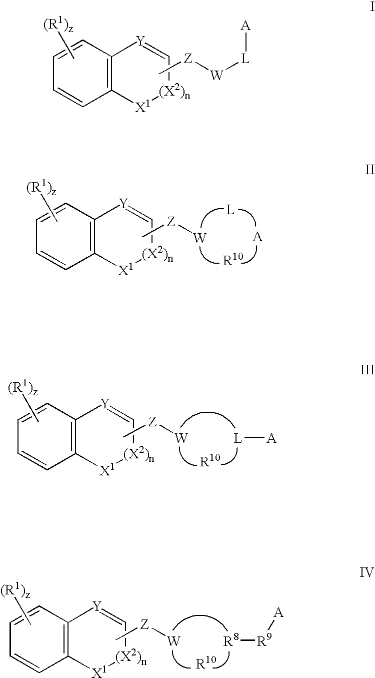
wherein R¹, z, X¹, X², W, Y, Z, n, L, A, R⁸, R⁹ and R¹⁰ have the same meaning as that defined in claim 1.

20. A method according to claim 19, wherein the condensation is performed via the formation of the acyl chloride of the compound of Formula LXIV and then by the coupling of said acyl chloride with the compound of Formula LXV, LXVI, LXVII or LXVIII, wherein W is N.

21. A method according to claim 20, wherein the condensation is performed using a suitable coupling agent, in a suitable solvent, in the presence of suitable base.

22. A method according to claim 21, wherein the suitable coupling agent is selected from the group comprising hydroxybenzotriazole, o-benzotriazol-1-yl-N,N,N′,N-4-tetramethyluronium hexafluorophosphate and the like.

23. A method according to claim 21, wherein the suitable solvent is selected from the group comprising dichloromethane, dimethylformamide and the like or a mixture thereof.

24. A method according to claim 21, wherein the suitable base is selected from the group comprising potassium carbonate, diisopropylethylamine, triethylamine, triisopropylamine and the like.

25. A method according to claim 21, wherein said base is used in an amount between 0.1 and 5.0 equivalents.

26. A compound obtainable by the method of claim 20.

27. (canceled)

28. A method for blocking an ion channel comprising contacting the ion channel with a compound according to claim 1.

29. The method according to claim 28 wherein the ion channel is an ion-channel of the Kv4 family of ion-channels.

30. The method according to claim 28 wherein the ion channel is the Kv1 family of ion-channels.

31. The method according to claim 28 wherein the ion channel is an ion-channel of the Kv4.3 family of ion-channels.

32. The method according to claim 28 wherein the ion channel is an ion-channel of the Kv1.5 family of ion-channels.

33. A method for the prevention and/or treatment of conditions or diseases associated with ion channels of the Kv4 family comprising administering to an individual in need of such treatment an effective amount of a compound according to claim 1.

34. The method according to claim 33, wherein said conditions or diseases associated with ion channels of the Kv4 family, preferably Kv4.3 ion channels, is selected from the group comprising cardiac disorders including arrhythmia, hypertension-induced heart disorders including hypertension-induced cardiac hypertrophy, disorders of the nervous system including epilepsy, stroke, traumatic brain injury, anxiety, insomnia, spinal cord injury, encephalomyelitis, multiple sclerosis, demyelinating disease, Alzheimer's disease and Parkinson's syndrome.

35. A method for the preparation of a medicament for the prevention and/or treatment of conditions or diseases associated with ion channels of the Kv1 family comprising administering to an individual in need of such treatment an effective amount of a compound according to claim 1.

36. The method according to claim 35, wherein said conditions or diseases associated with ion channels of the Kv1 family, preferably Kv1.5 ion channels, is selected from the group comprising cardiac disorders including arrhythmia, hypertension-induced heart disorders including hypertension-induced cardiac hypertrophy, disorders of the nervous system including epilepsy, stroke, traumatic brain injury, anxiety, insomnia, spinal cord injury, encephalomyelitis, multiple sclerosis, demyelinating disease, Alzheimer's disease and Parkinson's syndrome.

37. The method according to claim 33 wherein the conditions or diseases are cardiac disorders or disorders of the nervous system.

38. The method according to claim 36 wherein the conditions or diseases are cardiac disorders or disorders of the nervous system.

39. A pharmaceutical composition comprising a pharmaceutically acceptable excipient and a therapeutically effective amount of a compound according to claim 1.

40. A method for the treatment of conditions or diseases associated with ion channels of the Kv4 family, comprising administering to an individual in need of such treatment an effective amount of a pharmaceutical composition according to claim 39.

41. The method according to claim 40, wherein said conditions or diseases associated with ion channels of the Kv4 family is selected from the group comprising cardiac disorders including arrhythmia, hypertension-induced heart disorders including hypertension-induced cardiac hypertrophy, disorders of the nervous system including epilepsy, stroke, traumatic brain injury, anxiety, insomnia, spinal cord injury, encephalomyelitis, multiple sclerosis, demyelinating disease, Alzheimer's disease and Parkinson's syndrome.

42. A method for the treatment of conditions or diseases associated with ion channels of the Kv1 family, preferably the Kv1.5 ion channel, comprising administering to an individual in need of such treatment an effective amount of a pharmaceutical composition according to claim 39.

43.-45. (canceled)

46. Method of treating cardiac disorders comprising administrating to an individual in need of such treatment a pharmaceutical composition according to claim 39.

47. Method of treating disorders of the nervous system comprising administrating to an individual in need of such treatment a pharmaceutical composition according to claim 39.
